(19) United States
(12) Patent Application Publication
(10) Pub. No.: US 2014/0303010 A1
(43) Pub. Date: Oct. 9, 2014

(54) Methods and Compositions for Species-Specific Kinome Microarrays

(71) Applicant: University of Saskatchewan, Saskatoon (CA)

(72) Inventors: Brett Trost, Saskatoon (CA); Anthony Kusalik, Saskatoon (CA); Scott Napper, Saskatoon (CA); Ryan Arsenault, College Station, TX (US); Philip Griebel, Saskatoon (CA)

(21) Appl. No.: 14/346,490

(22) PCT Filed: Sep. 21, 2012
(86) PCT No.: PCT/CA2012/000893

§ 371 Date: Mar. 21, 2014

Related U.S. Application Data
(60) Provisional application No. 61/537,941, filed on Sep. 22, 2011.
(60) Provisional application No. 61/619,902, filed on Apr. 3, 2012.

(30) Foreign Application Priority Data
Jun. 24, 2012 (IB) PCT/IB2012/001254

(51) Int. Cl. G01N 33/68; G06F 19/22

(52) U.S. Cl. CPC G01N 33/6818; G06F 19/22
USPC 506/7; 506/30; 506/18; 702/20

(57) ABSTRACT

A method of preparing a species-specific phosphorylation site peptide array for a target organism comprising: a) selecting a plurality of known non-target organism (NTO) phosphorylation site sequences and cognate known NTO phosphorylation polypeptide sequences from one or more NTO, each of the known NTO phosphorylation site sequences comprising at least 5 residues and less than 30 residues; b) identifying a matching target organism (TO) phosphorylation site sequence and cognate TO phosphorylation polypeptide sequence for one or more of the known NTO phosphorylation site sequences; c) determining the matching TO phosphorylation site sequences that correspond to orthologue polypeptides of the cognate known NTO phosphorylation polypeptide sequences; d) selecting the matching TO phosphorylation site sequences determined to correspond to orthologue polypeptides for inclusion on the array; wherein the matching TO phosphorylation site sequences that correspond to orthologue polypeptides are determined by calculating, for each matching phosphorylation site sequence identified in b), a similarity value between the TO phosphorylation polypeptide sequence corresponding to the TO phosphorylation site sequence and a TO polypeptide sequence matching the cognate known NTO polypeptide sequence.

This application is a PCT claiming priority to U.S. provisional application 61/537,941 filed Sep. 22, 2011, US Provisional application filed Apr. 3, 2012 and PCT application PCT/IB2012/001254 filed Jun. 24, 2012, all of which are herein incorporated by reference.

### FIELD OF THE DISCLOSURE

The disclosure relates to methods for making species-specific phosphorylation site databases and arrays and species-specific kinome arrays.

### BACKGROUND OF THE DISCLOSURE

Protein phosphorylation is believed to be the most widespread mechanism of cellular signalling, with approximately one-third of all proteins in the eukaryotic cell estimated to undergo this post-translational modification (Johnson and Hunter, 2005). A recently developed technology for studying phosphorylation-mediated cellular signalling is the kinome microarray. Each spot on a kinome microarray contains a peptide representing a phosphorylation site (the actual phosphorylated residue, and several surrounding residues) from a given protein. These peptides are capable of being phosphorylated with similar kinase-catalyzed kinetics as the corresponding intact protein (Zetterqvist et al., 1976; Kemp et al., 1977). First proposed and tested in 2002 (Houseman and Mrksich, 2002; Houseman et al., 2002), kinome microarrays have since been used to study signalling in a number of biological systems (e.g. Löwenberg et al., 2005; Sikkema et al., 2009; Schrage et al., 2009).

The abundance of phosphorylation data for human, rat, and mouse in online databases like PhosphoSitePlus (Hornbeck et al., 2004) makes it relatively straightforward to design kinome microarrays for studying these species. Unfortunately, little phosphorylation data are available for other species.

### SUMMARY OF THE DISCLOSURE

A method of preparing one or more species-specific phosphorylation site database entries for a target organism comprising:

a) selecting a first known non-target organism (NTO) phosphorylation site sequence of a first non-target organism, the first known NTO phosphorylation site sequence comprising at least 5 residues and less than 30 residues and/or 30 or fewer residues;

b) obtaining for the first known NTO phosphorylation site sequence a first cognate known NTO phosphorylation polypeptide sequence corresponding to the first known NTO phosphorylation site sequence, the cognate known NTO phosphorylation polypeptide sequence comprising the first known NTO phosphorylation site sequence;

c) identifying a matching target organism (TO) phosphorylation site sequence for the first known NTO phosphorylation site sequence;

d) obtaining for the matching TO phosphorylation site sequence a cognate TO phosphorylation polypeptide sequence corresponding to the matching TO phosphorylation site sequence, the cognate TO phosphorylation polypeptide sequence comprising the matching TO phosphorylation site sequence;

e) determining a plurality of output values, each output value indicative of a degree of matching between the TO phosphorylation site sequence and the NTO phosphorylation site sequence; and

f) determining a similarity value between the first known NTO phosphorylation polypeptide sequence and the cognate TO phosphorylation polypeptide sequence, wherein the similarity value provides an indication of whether the first known NTO phosphorylation polypeptide sequence and the cognate TO phosphorylation polypeptide sequence are orthologues of each other.

In an embodiment, identifying a matching TO phosphorylation site sequence comprises:

a) retrieving a proteome of the target organism;

b) creating a dataset of target organism polypeptide sequences using the retrieved proteome of the target organism; and

c) querying the dataset of target organism polypeptide sequences.

In another embodiment, a processor executes a software program to retrieve the proteome of the target organism from an electronic database of protein sequence data and wherein the dataset of proteins of the target organism is a BLAST database created using the makeblastdb program.

In yet another embodiment, identifying a matching TO phosphorylation site sequence comprises:

a) comparing the first known NTO phosphorylation site sequence against a plurality of sequences of residues of the dataset of target organism proteins; and

b) determining the sequence of the plurality of sequences of residues of the dataset of target organisms proteins having the most number of identical residues as the NTO phosphorylation site sequences as the matching TO phosphorylation site sequence.

In an embodiment, the identifying of the matching TO phosphorylation site sequence comprises running a blastp search using the first known NTO phosphorylation site sequence as the query and the dataset of target organism proteins as the queried database.

In another embodiment, the plurality of output values comprises one or more of: a sequence difference, a non-conservative sequence difference, a matching TO phosphorylation site, a 9-mer sequence difference, and a 9-mer non-conservative sequence difference.

In yet another embodiment, the sequence difference is equal to the difference between the number of residues in the first known NTO phosphorylation site sequence and the number of identical residues between the first known NTO phosphorylation site sequence and the matching TO phosphorylation site sequence;

wherein the non-conservative sequence difference is equal to the difference between the number of residues in the first known NTO phosphorylation site sequence and the sum of the number of identical residues between the first known NTO phosphorylation site sequence and the hit sequence and the number of residues of the hit sequence that are conservative substitutions of the corresponding residue of the first known NTO phosphorylation site sequence.
wherein the matching TO phosphorylation site corresponds to a start position of the TO phosphorylation site sequence in the cognate TO phosphorylation polypeptide sequence;
wherein the 9-mer sequence difference is equal to the number of sequence differences in the count of positions where the two residues are different in a gapless alignment between a 9-amino-acid long peptide corresponding to the first known NTO phosphorylation site sequence and a 9-amino-acid long peptide corresponding to the matching TO phosphorylation site sequence;
and wherein the 9-mer non-conservative sequence difference is equal to the number of non-conservative sequence differences in the count of positions where the two residues have a non-positive score in a gapless alignment between the 9-amino-acid long peptide corresponding to the first known NTO phosphorylation site sequence and the 9-amino-acid long peptide corresponding to the matching TO phosphorylation site sequence.

In an embodiment, determining of the similarity value comprises:

a) retrieving a proteome of the first known non-target organism;

b) creating a dataset of first known NTO phosphorylation polypeptide sequences using the retrieved non-target organism proteome;

c) comparing the first known NTO phosphorylation polypeptide sequence to each of the TO phosphorylation polypeptide sequences of the dataset of TO phosphorylation polypeptide sequences to generate a plurality of TO dataset similarity values;

d) identifying a best TO dataset similarity value (E¹_{B}) from the plurality of TO dataset similarity values and identifying a first TO dataset similarity value (E¹_{F}) of the match between the first known NTO phosphorylation polypeptide sequence (Q_{F}) and the cognate TO phosphorylation polypeptide sequence (H_{F}) from the plurality of TO dataset similarity values;

e) comparing the TO phosphorylation polypeptide sequence to each of the first known NTO phosphorylation polypeptide sequences in the dataset of first known NTO phosphorylation polypeptide sequences to generate a plurality of NTO dataset similarity values;

f) identifying a best NTO dataset similarity value (E²_{B}) from the plurality of NTO dataset similarity values and identifying a first NTO dataset similarity value (E²_{F}) of the match between the first known NTO phosphorylation polypeptide sequence (Q_{F}) and the cognate TO phosphorylation polypeptide sequence (H_{F}) from the plurality of NTO dataset similarity values; and

g) if the first TO dataset similarity value equals the best TO dataset similarity value and if the first NTO dataset similarity value equals the best NTO dataset similarity value, determining the first known NTO phosphorylation polypeptide sequence and the cognate TO phosphorylation polypeptide sequence are orthologues of each other.

In an embodiment, one or more of the similarity values comprises an E-value. In another embodiment, the E-value is selected at less than 10⁻³.

In an embodiment, the method comprises:
a) identifying the best TO similarity value comprises running a blastp search using the first cognate known NTO phosphorylation polypeptide sequence as the query and the dataset of TO proteins as the queried database to generate a plurality of TO dataset E-values, wherein the smallest E-value of the plurality of TO dataset E-values is identified as a best TO dataset E-value;
b) identifying the best NTO dataset similarity value comprises running a blastp search using the cognate TO phosphorylation polypeptide sequence as the query and the dataset of NTO proteins as the queried database to generate a plurality of NTO dataset E-values, wherein the smallest E-value of the plurality of NTO dataset E-values is identified as a best NTO dataset E-value; and
c) if the first TO dataset E-value equals the best TO dataset E-value and if the first NTO dataset E-value equals the best NTO dataset E-value, determining the first known NTO phosphorylation polypeptide sequence and the cognate TO phosphorylation polypeptide sequence are orthologues of each other.

In an embodiment, each of the first known NTO phosphorylation site sequence and the matching TO phosphorylation site sequence is at least 8 residues and less than 15 residues in length.

In yet another embodiment, wherein a plurality of output values is displayed. In an embodiment, the plurality of output values is outputted electronically in a delimited plain text format.

A further aspect includes a method of making a species-specific array comprising selecting a plurality of matching target organism phosphorylation site sequences according to a method described herein, synthesizing a plurality of peptides each peptide comprising a sequence of one of the matching target organism phosphorylation site sequences and attaching the plurality of peptides to a substrate surface.

A further aspect includes a plurality of peptides, each of which comprises a sequence of about 5 to about 100 amino acids, for example about 5 to about 50 amino acids or about 5 to about 30 amino acids, wherein each sequence comprises a contiguous sequence of at least 5 amino acids present in a peptide sequence selected from the group of SEQ ID NOs: 1 to 292, wherein the contiguous sequence comprises a chicken phosphorylation site sequence.

In an embodiment, the plurality of peptides comprises about 5, 10, 15, 20, 25, 50, 75, 100, 125, 150, 175, 200, 225, 250, 275 or 292 peptides each comprising all or part of a sequence selected from SEQ NO: 1-292.

Yet a further aspect includes a species-specific array comprising a plurality of peptide attached to a support surface, each peptide comprising a sequence of about 5 to about 100 amino acids, for example about 5 to about 50 amino acids or about 5 to about 30 amino acids or about 8 to about 15 amino acids, wherein the sequence is a matching target organism phosphorylation site sequence selected as described herein, wherein the similarity is below a preselected threshold.

In an embodiment, the array plurality of peptides comprises at least 100, 200, or 292 matching target organism phosphorylation site sequences.

In an embodiment, the array further comprises one or more negative control peptides and/or one or more positive control peptides.

In a further embodiment, the array is a chicken species array and the plurality of peptides are chicken peptides.

In an embodiment, the plurality of array peptides comprises about 5, 10, 15, 20, 25, 50, 75, 100, 125, 150, 175, 200, 225, 250, 275 or 292 peptides each comprising all or part of a sequence selected from SEQ NO: 1-292.

In another embodiment, for each of a plurality of the array peptides, each peptide is 8-15 contiguous amino acids of a sequence selected from SEQ ID NO: 1-292

In yet another embodiment, a plurality of the array peptides is spotted in replicates of 2, 3, 4, 5, 6, 7, 8, or 9 or more.

Another aspect includes a method of determining kinome activity of a test sample comprising:

a) incubating an array described herein with the test sample to provide a test array and optionally incubating a second array described herein with a comparator sample to provide a comparator array; and

b) measuring a phosphorylation level signal intensity for each of the plurality of peptides for the test array and optionally the compartor array wherein the phosphorylation level signal intensity results from the interaction of the sample with each of the plurality of peptides;

wherein the kinome activity is determined by identifying an increased or decreased phosphorylation level of one or more of the plurality of peptides on the test array compared to the comparator or an internal control.

A further aspect includes a method of determining a phosphorylation profile of a test sample comprising:

a) incubating a species-specific array described herein with the test sample to provide a test array; and

b) measuring a phosphorylation level signal intensity for each of the plurality of peptides for the test array providing a test array phosphorylation profile, wherein the phosphorylation level signal intensity results from the interaction of the sample with each of the plurality of peptides.

In an embodiment the method further comprises incubating a species-specific array with a comparator sample to provide a comparator array; measuring a phosphorylation level signal intensity for each of the plurality of peptides for the comparator array wherein the phosphorylation level signal intensity results from the interaction of the sample with each of the plurality of peptides and comparing the test array phosphorylation profile to the comparator phosphorylation profile to detect one or more differentially phosphorylated peptides.

In an embodiment, the comparator sample is a control that can correspond to background. In an embodiment, the comparator sample is a test sample,

A further aspect includes a non-transitory computer-readable storage medium upon which a plurality of instructions are stored, the instructions for performing the steps of:

a) querying a dataset comprising a plurality of target organism (TO) polypeptide sequences with a selected plurality of known NTO phosphorylation site sequences (query phosphorylation site sequences) to identify for each of the plurality a matching TO phosphorylation site sequence;

b) obtaining for each of the matching TO phosphorylation site sequences a cognate TO phosphorylation polypeptide sequence corresponding to the matching TO phosphorylation site sequence, the cognate TO phosphorylation polypeptide sequence comprising the matching TO phosphorylation site sequence;

c) determining a plurality of output values, one or more of the output values being indicative of a degree of matching between the TO phosphorylation site sequence and the NTO phosphorylation site sequence; and

d) determining a similarity value between the first known NTO phosphorylation polypeptide sequence and the cognate TO phosphorylation polypeptide sequence, wherein the similarity value provides an indication of whether the first known NTO phosphorylation polypeptide sequence and the cognate TO phosphorylation polypeptide sequence are orthologues of each other.

In an embodiment, the instructions are further for performing the steps of displaying the matching TO phosphorylation site sequences and/or cognate TO sequence accession numbers when the similarity value is below a preselected threshold.

Another aspect includes a system for preparing one or more species-specific phosphorylation site database entries for a target organism, the system comprising:

a) a memory for storing a plurality of instructions; and

b) a processor coupled to the memory for:
i) obtaining for a first known non-target organism (NTO) phosphorylation site sequence of a first non-target organism, the first known NTO phosphorylation site sequence comprising at least 5 residues and less than 30 residues, a first cognate known NTO phosphorylation polypeptide sequence corresponding to the first known NTO phosphorylation site sequence, the cognate known NTO phosphorylation polypeptide sequence comprising the first known NTO phosphorylation site sequence;
ii) identifying a matching target organism (TO) phosphorylation site sequence for the first known NTO phosphorylation site sequence;
iii) obtaining for the matching TO phosphorylation site sequence a cognate TO phosphorylation polypeptide sequence corresponding to the matching TO phosphorylation site sequence, the cognate TO phosphorylation polypeptide sequence comprising the matching TO phosphorylation site sequence;
iv) determining a plurality of output values, one or more of the output values being indicative of a degree of matching between the TO phosphorylation site sequence and the NTO phosphorylation site sequence; and
v) determining a similarity value between the first known NTO phosphorylation polypeptide sequence and the cognate TO phosphorylation polypeptide sequence, wherein the similarity value provides an indication of whether the first known NTO phosphorylation polypeptide sequence and the cognate TO phosphorylation polypeptide sequence are orthologues of each other.

A further aspect includes a kit comprising a plurality of peptides described herein, an array described herein, and/or a kit control and/or package housing the peptides, array and/or kit control.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the disclosure will now be discussed in relation to the drawings in which:

FIG. 1 is a flowchart illustrating the general operational steps of an exemplary embodiment for preparing one or more species-specific phosphorylation site database entries for a target organism.

FIG. 2 is a flowchart illustrating the general operational steps of an exemplary embodiment for identifying a matching target organism phosphorylation site sequence for the first known NTO phosphorylation sequence.

FIG. 3 is a flowchart illustrating the general operational steps of Design Array for PhosPhoryLation Experiments (DAPPLE).

FIG. 4 is a schematic diagram illustrating the flow of data generated from a single phosphorylation site data according to DAPPLE.

FIG. 5 is a schematic diagram illustrating the flow of data generated from a target proteome according to DAPPLE.

FIG. 6 is a schematic diagram illustrating the flow of data generated from some of the operational steps of DAPPLE.

FIG. 7 is a schematic diagram illustrating the flow of data generated from some of the operational steps of DAPPLE for determining whether Q_{F} 413 and H_{F} 436 are reciprocal BLAST hits.

FIG. 8: Cluster Analysis of Kinome Datasets of Thigh and Breast Samples of Temperature Stressed Birds. Kinome data sets were subjected to hierarchical clustering analysis. “Average Linkage+(1−Pearson Correlation)” was used for clustering both the animal-treatments (in vertical direction) and the peptides (in horizontal direction). The animal codes are indicated right below the corresponding treatment names under the heat map.

FIG. 9: Cluster Analysis of Kinome Datasets of Thigh and Breast Samples of Representative Temperature Stressed Birds. Kinome data sets were subjected to hierarchical clustering analysis. “Average Linkage+(1−Pearson Correlation)” was used for clustering both the animal-treatments (in vertical direction) and the peptides (in horizontal direction). The animal codes are indicated right below the corresponding treatment names under the heat map.

FIG. 10: Differentially Modified Peptides Amongst the Different Tissues and Treatment Conditions. A) Thigh B) Breast

FIG. 11. Hierarchical clustering results for control, heat-treated, or cold-treated chicken breast and thigh samples. The clustering was done using Pearson correlation as the distance metric and average linkage as the linkage method.

### DETAILED DESCRIPTION OF THE DISCLOSURE

The kinome microarray is a relatively new technology for studying phosphorylation-mediated cellular signalling. Other than for human, rat, and mouse, relatively little phosphorylation data are available for most organisms, making it difficult to design kinome microarrays suitable for studying them. Recently a protocol was developed for leveraging known phosphorylation sites from one organism to identify putative sites in a different organism. While effective, this procedure is time-consuming, tedious, and cannot feasibly make use of even a small fraction of the known phosphorylation sites. Methods and systems for identifying putative phosphorylation sites in an organism of interest are provided. In an embodiment, the disclosure includes a collection of Perl scripts called Design Array for PhosPhoryLation Experiments (DAPPLE) that automates the identification of putative phosphorylation sites in an organism of interest, improving and accelerating the process of designing kinome microarrays for example species other than human, rat, and mouse.

### DEFINITIONS

As used in this specification and the appended claims, the singular forms “a”, “an” and “the” include plural references unless the content clearly dictates otherwise.

The term “accession number” as used herein refers to a code such as a Genbank accession number that uniquely identifies a particular polypeptide sequence (e.g. protein or part thereof) and/or DNA encoding said polypeptide or part thereof.

The term “corresponds to” as used herein means in the context of a sequence and a second sequence from the same species, corresponds to sequences that derive from the same protein e.g. a phosphorylation site sequence and a full length polypeptide which contains the phosphorylation site sequence. Similarly, regarding a first sequence and a “corresponding protein identifier” from the same species refers to a protein identifier such as an accession number that identifies the same protein as contains the first sequence. As another example, reference to a “matching target organism (TO) phosphorylation site sequence that corresponds to an orthologue polypeptide of the known non-target organism (NTO) phosphorylation polypeptide sequence” means that the matching TO phosphorylation site sequence is found in the same protein which is an orthologue of the NTO phosphorylation polypeptide sequence protein.

The term “E-value” or “Expect value” as used herein has the same meaning as provided by National Center for Biotechnology Information (NCBI) and means a parameter that describes the number of hits one can “expect” to see by chance when searching a database of a particular size. It decreases exponentially as the Score (S) of the match increases. For example, an E-value of 1 assigned to a hit can be interpreted as meaning that in a database of the current size one might expect to see 1 match with a score equal to or greater than the score actually observed simply by chance. The smaller the E-value, or the closer it is to zero, the more “significant” the match is. However, keep in mind that virtually identical short alignments have relatively high E-values. This is because the calculation of the E-value takes into account the length of the query sequence. These high E-values make sense because shorter sequences have a higher probability of occurring in the database purely by chance.

The phrase “cognate TO phosphorylation polypeptide sequence” and/or H_{F} as used herein means a polypeptide sequence that comprises the TO phosphorylation site sequence and has for example the same accession number as the TO phosphorylation site sequence e.g. they relate to the same protein. The cognate TO phosphorylation polypeptide sequence is longer in length than the TO phosphorylation site sequence, and can for example comprise the full length sequence of the protein or a part thereof. For example, each TO phosphorylation site sequence is identified by screening a database of polypeptides and accordingly its sequence is contained within a protein and is understood to correspond to the protein from which it derives. Accordingly, the TO phosphorylation site sequence and the TO phosphorylation polypeptide sequence correspond to the same protein, for example as defined by a protein identifier such as an accession number. The TO phosphorylation polypeptide sequence can for example consist of at least 10%, 20%, 30%, 40%, 50%, 60%, 70%, 80%, 90% or the full length of the protein

The phrase “cognate known NTO phosphorylation polypeptide sequence” and/or Q_{F} as used herein means a polypeptide sequence that comprises the NTO phosphorylation site sequence and has for example the same accession number as the NTO phosphorylation site sequence e.g. they relate to the same protein. The cognate NTO phosphorylation polypeptide sequence is longer in length than the NTO phosphorylation site sequence and can for example comprise the full length sequence of the protein or a part thereof. For example, NTO phosphorylation site sequence is identified by screening a database of polypeptides and accordingly its sequence is contained within a protein and is understood to correspond to the polypeptide/protein from which it derives. Accordingly, the NTO phosphorylation site sequence and the NTO phosphorylation polypeptide sequence correspond to the same protein, for example as defined by a protein identifier such as an accession number. The NTO phosphorylation polypeptide sequence can for example consist of at least 10%, 20%, 30%, 40%, 50%, 60%, 70%, 80%, 90% or the full length of the protein.

The term “low-throughput references” as used herein indicates the number of references to peer-reviewed papers in which the authors employed low-throughput biological techniques (techniques capable of analyzing only one or a few phosphorylation sites at a time) to characterize the known phosphorylation site and the term “high-throughput references” indicates the number of references to peer-reviewed papers in which the authors used high-throughput biological techniques (techniques capable of analyzing many phosphorylation sites at a time, like mass spectrometry) to characterize the known phosphorylation site. The number of low-throughput references and high-throughput references are provided for example by the PhosphoSitePlus database for each known phosphorylation site.

The phrase “matching TO phosphorylation site sequence” and/or “H” refers to a TO polypeptide sequence consisting of at least 5 residues and less than 30 residues or at least 5 residues and 30 or fewer residues (or corresponding nucleotide residues) that has the highest similarity of a plurality of TO polypeptide sequences, for example the highest percent identity of the TO proteome polypeptides over for example a portion of H, with a corresponding NTO phosphorylation site sequence, and which is identified for example by querying a TO polypeptide database with a known NTO phosphorylation site sequence query. As an example, a matching TO phosphorylation site sequence may have 0, 1, 2, 3, 4, 5 or 6 or more residues that are different from the NTO query sequence, for example depending on the length of the query sequence. The phosphorylation site or site phosphorylated in H is H_{C}, the description of the protein/polypeptide comprising H can be H_{D}, H_{L} can be the number or residues in H (e.g. length) and H_{A} can be the accession number associated with H, for example a Genbank accession number.

The phrase “matching TO phosphorylation polypeptide sequence” as used herein refers to a TO polypeptide sequence consisting of all or part of the corresponding protein and that has the highest similarity of a plurality of TO polypeptide sequences, for example the highest percent identity of the TO proteome polypeptides, with a corresponding NTO phosphorylation polypeptide sequence, which is identified for example by querying a TO polypeptide database with a known NTO phosphorylation polypeptide sequence. As an example, a first polypeptide sequence (e.g. a TO phosphorylation polypeptide sequence) will match a second polypeptide sequence (e.g. a NTO phosphorylation polypeptide sequence) if the E-value is less than a preselected value, for example 10⁻³.

As used herein, “NTO phosphorylation site sequence” and “Q” as used herein, refers to a known phosphorylation site sequence, which can be for example from 5 amino acid residues (or corresponding nucleotide residues e.g. 15 nucleotides) to about and including 30 amino acids (or corresponding nucleotides e.g. about 90 nucleotides) and which is used as a “query” sequence in the methods described. The NTO phosphorylation site sequence can be any string of amino acids (or corresponding nucleotides) found in the NTO that is known (or suspected) of having a residue that is phosphorylated. For example, any string of any amino acids comprising at least one of “serine”, “threonine” or “tyrosine” or encoding at least one of these, can be suspected of having a residue that is phosphorylated. The phosphorylation residue can be for example in the middle position of Q (e.g. amino acid residue 8 for a 15 amino acid query sequence) or for example any position. The phosphorylation site or site phosphorylated in Q is Q_{C}, the organism can be Q_{O} and the description of the protein/polypeptide comprising Q can be Q_{D}, Q_{L} can be the number or residues in Q (e.g. length) and Q_{A} can be the accession number associated with Q, for example a Genbank accession number.

The term “non-conservative sequence change” as used herein means when referring to an amino acid sequence, a corresponding (e.g. aligned) amino acid residue between a first sequence and a second sequence, wherein the amino acid residue in the first sequence is not a conservative or semi-conservative substitution of the corresponding amino acid in the second sequence, e.g. the polarity of the amino acid residue (or other biochemical property) in the first sequence is markedly different from the polarity (or other biochemical property) of the corresponding amino acid residue in the second sequence. For example, replacing one amino acid residue with another having similar hydrophobicity and/or molecular side chain bulk can be considered a conservative sequence change. As an example, blastp as a default uses the substitution matrix BLOSUM62 to assess conservative and non-conservative substitutions. However the user can specify a substitution matrix that fits a particular sequence comparison context. As examples, alanine, serine and threonine are considered conservative substitutions, as are aspartic acid and glutamic acid, or asparagine and glutamine. Similarly, arginine and lysine are commonly considered conservative substitutions, as are isoleucine, leucine, methionine and valine. Phenylalanine, tyrosine and tryptophan are also considered conservative changes. Non-conservative changes would include for example alanine and aspartic acid; serine and aspartic acid; or arginine and valine.

Homologues are proteins that have shared evolutionary ancestry. Most homologues are orthologues or paralogues. Orthologues are proteins from different species that evolved from a common ancestral gene by speciation, and which typically retain the same function in the course of evolution. The term “orthologous polypeptide” refers to a protein that is the orthologue of the protein in another species. Paralogues are proteins in the same species, one of which resulted from a genetic duplication of the other).

As used herein, “peptide array” or “array” means a plurality of peptides coupled to a support, wherein each peptide comprises a putative or known phosphorylation motif, e.g. a phosphorylation site sequence. An array can be for example a two-dimensional arrangement of a plurality of peptide molecules, each peptide comprising a known or putative phosphorylation site, attached on a support surface such as a slide or a bead. Arrays are generally comprised of regular, ordered peptide molecules, as in for example, a rectilinear grid, parallel stripes, spirals, and the like, but non-ordered arrays may be advantageously used as well. The arrays generally comprise in the range of about 2 to about 3000 different peptides, more typically about 2 to about 1,200 different peptides. The array can for example comprise 25, 50, 100, 150, 200, 250, 300, 400, 500, 1000, 1200 or more different peptides, spotted in a single replicate, or in replicates of 2, 3, 4, 5, 6, 7, 8, or 9 or greater. For example, depending on the dataset to be obtained, the peptide array can comprise peptides with known phosphorylation motifs (e.g., phosphorylation site sequences), optionally phosphorylation motifs for proteins that are found in a signaling pathway or related pathways. Such peptide arrays can be useful for deciphering peptides phosphorylated or signaling pathways activated by a stressor such as an infectious agent or a macromolecule. The peptide molecules comprise for examples peptides or parts thereof, selected from the peptides listed in Table 6.

The term “attached,” as in, for example, a support surface having a peptide molecule “attached” thereto, includes covalent binding, adsorption, and physical immobilization. The terms “binding” and “bound” are identical in meaning to the term “attached.” The peptide can for example be attached via a flexible linker.

Alternatively, the peptide array can comprise random peptide sequences comprising putative phosphorylation sites wherein the plurality of peptides or a subset thereof comprises at least one of a serine, threonine or tyrosine residue.

The term “peptide molecule” or “peptide” as used herein includes a molecule comprising a chain of 5 or more amino acids comprising optionally a known or putative phosphorylation site or optionally in the case of a control peptide, the lack of a phosphorylation site. A peptide in the context of a peptide array typically comprises a peptide having from about 5 to about 21 amino acid residues or any number in between. The peptide can also be longer, for example up to 30 amino acids, up to 50 amino acids or up to 100 amino acids. For example, the peptide can comprise a sequence listed in Table 6 and additional surrounding cognate protein sequence which can be identified according to the corresponding accession number. An amino acid linker can also be included. A polypeptide and/or protein can comprise any length of amino acid residues. In an embodiment, the term “peptide” for example when used as a probe on an array refers to a peptide comprising at least 5 residues and less than 30 residues and/or 30 or fewer residues.

The phrase “phosphorylation site sequence” means a polypeptide sequence consisting of at least 5 residues and less than 30 residues and/or 30 or fewer residues (for example 15 residues) and that comprises at least one serine, threonine or tyrosine residue phosphorylatable by one or more kinases.

For example, the peptide or phosphorylation site sequence can be 5, 6, 7, 8, 9, 10, 11, 12, 13, 14, 15, 16, 17, 18, 19, 20, 21, 22, 23, 24, 25, 26, 27, 28, 29 or 30 residues.

As used herein, the term “plurality of peptides” means at least 2, for example at least 3 peptides, at least 4 peptides, at least 5 peptides, at least 10, at least 15, at least 25 peptides, at least 50 peptides, at least 100 peptides, at least 200 peptides, at least 300 peptides, at least 400, at least 500 or at least 1000 or any number in between 2 and 1000.

The term “proteome” as used herein refers to the set of polypeptides expressed by a particular organism, optionally under control or test conditions. The term “subproteome” refers to a subset of the set of polypeptides comprised in a proteome, for example, a subset expressed under a specified test condition e.g. stimulated, or a subset that corresponds to a group of proteins e.g. immune system proteins.

The term “phosphorylation profile” or “subject phosphorylation profile” as used herein refers to, for a plurality (e.g. at least 2, for example 5) of peptides and/or their corresponding proteins, phosphorylation signal intensities detectable after contacting a sample from a subject with the plurality of peptides under conditions that permit peptide phosphorylation as would be known to a person skilled in the art (e.g. temperature, buffer constituents, presence of ATP and/or other suitable ATP source etc.). The plurality of peptides optionally comprises at least 2, at least 3, at least 4, at least 5, or more of the peptides listed in Table 6, including for example any number of peptides between 2 and 292.

The term “determining a phosphorylation level” or “determining a phosphorylation profile” as used herein means the contacting a reagent such as a peptide, or a plurality of peptides, to a sample, for example a sample of the subject chicken and/or a control sample, for ascertaining or measuring quantitatively, semi-quantitatively or qualitatively the amount of peptide phosphorylation signal intensity. For example, the plurality of peptides can be comprised in an array (e.g. on a slide or beads) as described herein and phosphorylation specific stains such as fluorescent ProQ Diamond Phosphoprotein Stain (Invitrogen) and Stains-All” (1-ethyl-2-[3-(3-ethylnaphtho [1,2]thiazolin-2 ylidene)-2-methylpropenyl]-naphtha [1,2]thiazolium bromide) and/or labeled ATP such as radiolabelled ATP can be used to detect phosphorylation. The phosphorylation signal can be detected by a number of methods known in the art such as using phosphospecific antibodies directly or indirectly labeled and/or using a method disclosed herein. For example a phosphospecific detection agent such as an antibody, for example a labeled antibody, which specifically binds the phosphorylated forms of peptides, can be used for example to detect relative or absolute amounts of peptide phosphorylation.

The term “difference in the level” as used herein in comparison to a control (e.g. or to a phenotype reference phosphorylation profile) or an internal control refers to a measurable difference in the level or quantity of peptide phosphorylation in a test sample, compared to the control that is of sufficient magnitude to allow assessment, for example of a statistically significant difference. For example, a difference in a level of peptide phosphorylation is detected if a ratio of the level in a test sample as compared with a control is greater than 1.2. For example, a ratio of greater than 1.3, 1.4, 1.5, 1.6, 1.7, 2, 2.5 or 3 or more and/or has a p-value of less than 0.1, 0.05 or 0.01.

The term “phosphorylation level” as used herein in reference to a peptide phosphorylation refers to a phosphorylation signal intensity that is detectable or measurable in a sample and/or control.

The term “measuring” or “measurement” as used herein refers to the application of an assay to assess the presence, absence, quantity or amount (which can be an relative or absolute amount) of either a given substance within a subject-derived sample, including the derivation of qualitative or quantitative concentration levels of such substances.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions times 100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the blastn and blastp programs of Altschul et al., 1990, J. Mol. Biol. 215:403. BLAST nucleotide searches can be performed with the blastn nucleotide program parameters set, to default parameters or e.g., wordlength=28. BLAST protein searches can be performed with the blastp program parameters set to default parameters, or e.g., wordlength=3 to obtain amino acid sequences homologous to a polypeptide molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of blastp and blastn) can be used (see, e.g., the NCBI website). The percent identity between two sequences can be determined using techniques similar to those described above, with or without allowing gaps. In calculating percent identity, typically only exact matches are counted.

The phrase “similarity value” as used herein means a value that indicates that two sequences are likely orthologues based on the similarity of the sequences. The similarity value can for example be a reciprocal blast hit (RBH) value which for example is identified by taking your known sequence, BLAST searching it against the gene sequences of the target organism, taking the highest scoring hit (e.g. lowest E-value) and BLAST searching the hit against a database of gene sequences of the known organism to determine if the known sequence is the best match (e.g. lowest Evalue) and therefore putative ortholog; or an E-value, percent similarity or other similar value. The similarity value is in an embodiment an E-value which gives for example an indication of whether the blast hit is a homologue and/or orthologue. For example, when comparing two sequences, a small E-value, for example below a selected threshold, is indicative the sequences are likely orthologues and/or homologues. The smaller the E-value, the greater the likelihood of similarity. Correspondingly, a large E-value, for example above a selected threshold, indicates the two sequences are likely not orthologues. The larger the E-value the less likely the two sequences are orthologues. As another example, a high percentage identity can be indicative that the two sequences are orthologues. The higher the percentage identity, the greater the likelihood the two sequences are orthologues and the lower the percentage identity, the greater the likelihood the two sequences are not orthologues. Although percent identity can also be used, E-value is preferable as the E-value takes into account sequence length, database size, etc. In embodiments, where the similarity value is an E-value, the smaller the similarity value, the greater the likelihood the sequences are orthologues. In embodiments when comparing a similarity value to a preselected threshold, a person skilled in the art would understand that if other similarity parameters are used (e.g. other than E-value) such as percent identity where the larger the value the greater the likelihood two sequences are orthologues, the inverse number e.g. 1/(percent identity), can be used to compare to the preselected threshold e.g. such that a similarity value below a preselected threshold is indicative of the two sequences being orthologues.

The phrase “species-specific phosphorylation site” as used herein means a sequence of amino acid residues which comprise a known or putative phosphorylation site of a specific target organism. The species-specific phosphorylation sites are identified for example by comparison to known phosphorylation sites of another species in orthologous polypeptides.

The phrase “species-specific phosphorylation site database” as used herein means a plurality of polypeptide sequences and corresponding annotations of a particular organism, wherein each sequence comprises a putative phosphorylation site. The sequences and annotations can be digitized and stored for retrieval, for example on a storage medium.

As used herein “target organism” means the species for which the user wants to design a database or a kinome array.

The term “sample” as used herein means any biological fluid or tissue sample from a subject, or fraction thereof which can be assayed for kinase activity, including for example, a lysate of a part of an organism or cell population wherein the cell population is obtained from a subject. The sample can be an experimental sample treated with a stressor or a control that is optionally untreated or treated with a control treatment (e.g. vehicle only). Depending on the stressor, an appropriate control treatment can be a vehicle only treatment (e.g. stressor dissolution agent) or a control treatment that is similar in composition to the stressor treatment but lacking the specificity of the stressor. For example a control treatment for a macromolecule, such as a peptide or RNA that induces a sequence specific cell response, can comprise a scrambled macromolecule, e.g. sequence scrambled peptide or RNA molecule. Similarly an isotype control antibody can be used as a control treatment wherein the stressor is an antibody. Any population of cells can be treated. For example, the cell or population of cells can comprise subject cells from multiple subjects, each sample optionally corresponding to a different subject, wherein one or more subsets of cells from each subject are treated with a stressor, optionally in vivo (e.g. an animal challenge) or in vitro (e.g. ex vivo treated primary cells). The cells are optionally clonal cells (e.g. cell culture experiment) and comprise propagated cells under defined conditions. Wherein multiple stressors are being compared or when using cells from one or more subjects, a biological control dataset for the same subject and/or sample treatment is optionally obtained and optionally subtracted from an experimental dataset (e.g. a control dataset comprising phosphorylation signal intensities corresponding to an unstimulated level of kinase activity is subtracted from each treatment dataset).

The term “phenotype” as used herein means a physical, behavioural, developmental, physiological, or biochemical characteristic of an organism, determined by genetic makeup and/or environmental influences.

The term “reference phosphorylation profile” or “phenotype reference phosphorylation profile” as used herein refers to a suitable comparison profile, for example which comprises the phosphorylation characteristics of a plurality of peptides, for example selected from the peptides listed in Table 6, associated with a particular phenotype. The reference phosphorylation profiles are compared to subject phosphorylation profiles for a plurality of peptides). A subject can be classified by comparing to a phenotype reference phosphorylation profile, where the phenotype reference phosphorylation profile most similar to the subject profile is indicative that the subject is likely to express the phenotype associated with the phenotype reference phosphorylation profile.

The term “similar” in the context of a phosphorylation level as used herein refers to a subject phosphorylation level for a peptide that falls within the range of levels associated with a particular class. Accordingly, “detecting a similarity” refers to detecting a phosphorylation level (or levels) that falls within the range of levels associated with a particular class. In the context of a reference phosphorylation profile, a subject profile is “similar” to a reference phosphorylation profile associated with a phenotype if the subject profile shows a number of identities and/or degree of changes (e.g. in terms of direction of phosphorylation (increased or decreased) and/or magnitude) with the reference phosphorylation profile.

The term “most similar” in the context of a reference phosphorylation profile refers to a reference phosphorylation profile that shows the greatest number of identities and/or degree of changes with the subject phosphorylation profile.

The term “kit control” as used herein means a suitable assay standard or reference reagent useful when determining a phosphorylation level of a peptide, for example a peptide that known to be phosphorylated or not phosphorylated under the conditions of the assay or for example a peptide corresponding to a substrate of a kinase with constitutive activity.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

In understanding the scope of the present disclosure, the term “consisting” and its derivatives, as used herein, are intended to be close ended terms that specify the presence of stated features, elements, components, groups, integers, and/or steps, and also exclude the presence of other unstated features, elements, components, groups, integers and/or steps.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that “a,” “an,” and “the” include plural referents unless the content clearly dictates otherwise. The term “about” means plus or minus 0.1 to 50%, 5-50%, or 10-40%, preferably 10-20%, more preferably 10% or 15%, of the number to which reference is being made.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### II. Methods and Products

The embodiments of the systems and methods described herein may be implemented in hardware or software, or a combination of both. However, preferably, these embodiments are implemented in computer programs executing on programmable computers each comprising at least one processor, a data storage system (including volatile and non-volatile memory and/or storage elements), at least one input device, and at least one output device. For example and without limitation, the programmable computers may be a personal computer, laptop, workstation, or network of a plurality of computers. Program code is applied to input data to perform the functions described herein and generate output information. The output information is applied to one or more output devices, in known fashion.

Each program is preferably implemented in a high level procedural or object oriented programming and/or scripting language to communicate with a computer system. However, the programs can be implemented in assembly or machine language, if desired. In any case, the language may be a compiled or interpreted language. Each such computer program is preferably stored on a storage media or a device (e.g. ROM or magnetic diskette) readable by a general or special purpose programmable computer, for configuring and operating the computer when the storage media or device is read by the computer to perform the procedures described herein. The subject system may also be considered to be implemented as a computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer useable instructions may also be in various forms, including compiled and non-compiled code.

Disclosed herein are methods and products for identifying putative phosphorylation sites in a target organism.

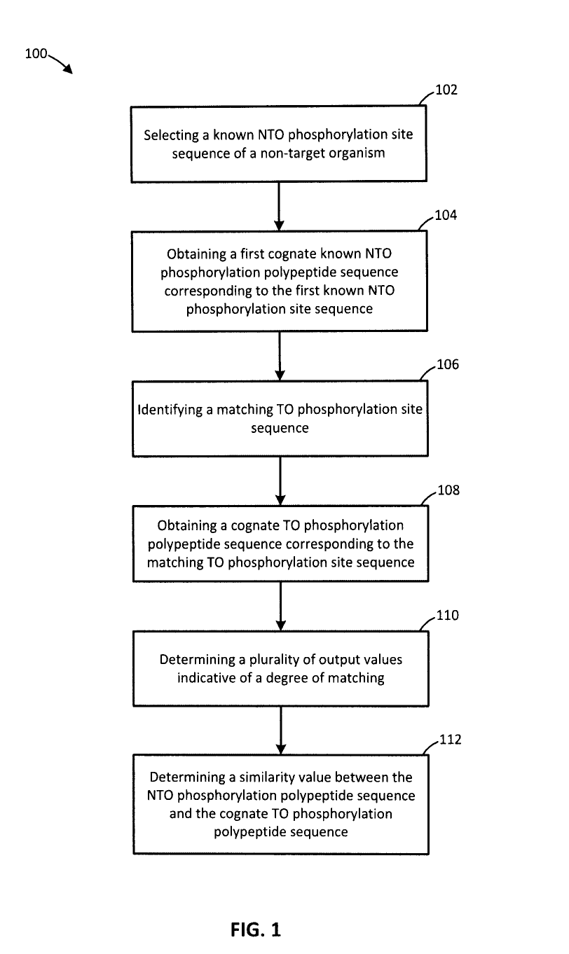

Referring now to FIG. 1, therein illustrated is a schematic diagram of a method 100 according to some exemplary embodiments.

In an aspect, the disclosure includes a method of preparing one or more species-specific phosphorylation site database entries for a target organism comprising:

e) at step 102 selecting a first known non-target organism (NTO) phosphorylation site sequence of a first non-target organism, the first known NTO phosphorylation site sequence comprising at least 5 residues and less than 30 residues and/or 30 or fewer residues;

f) at step 104 obtaining for the first known NTO phosphorylation site sequence a first cognate known NTO phosphorylation polypeptide sequence corresponding to the first known NTO phosphorylation site sequence, the cognate known NTO phosphorylation polypeptide sequence comprising the first known NTO phosphorylation site sequence;

g) at step 106 identifying a matching target organism (TO) phosphorylation site sequence for the first known NTO phosphorylation site sequence;

h) at step 108 obtaining for the matching TO phosphorylation site sequence a cognate TO phosphorylation polypeptide sequence corresponding to the matching TO phosphorylation site sequence, the cognate TO phosphorylation polypeptide sequence comprising the matching TO phosphorylation site sequence;

i) at step 110 determining a plurality of output values, one or more of the output values being indicative of a degree of matching between the TO phosphorylation site sequence and the NTO phosphorylation site sequence; and

j) at step 112 determining a similarity value between the first known NTO phosphorylation polypeptide sequence and the cognate TO phosphorylation polypeptide sequence, wherein the similarity value provides an indication of whether the first known NTO phosphorylation polypeptide sequence and the cognate TO phosphorylation polypeptide sequence are orthologues of each other.

In an embodiment, a database is populated with one or more values corresponding to the TO phosphorylation site sequence (e.g. when the first known NTO phosphorylation polypeptide sequence and the cognate TO phosphorylation polypeptide sequence are determined to be orthologues of each other).

The method may be repeated for a plurality of known non-target organism phosphorylation site sequences such that a plurality of database entries for the target organism can be prepared. The plurality of entries form a species-specific phosphorylation site database for the target organism which may then be used to facilitate the design of species-specific kinome microarrays.

In an embodiment, the first known non-target organism (NTO) phosphorylation site sequence is downloaded from a database, for example from PhosphoSitePlus Hornbeck, P. V., Chabra, I., Kornhauser, J. M., Skrzypek, E., and Zhang, B. (2004). PhosphoSite: A bioinformatics resource dedicated to physiological protein phosphorylation. Proteomics, 4(6), 1551-61. In an embodiment, the NTO phosphorylation site sequence is obtained from PhosphoSitePlus data files. Where the method is repeated for a plurality of known NTO phosphorylation site sequences, each of the NTO phosphorylation site sequences is downloaded from the database.

Depending on the database used for the download, the plurality of known non-target organism (NTO) phosphorylation site sequences may comprise duplicate phosphorylation site sequences from one or more NTO. For example, the PhosphoSitePlus data file contains entries with identical sequences (from different organisms).

In an embodiment, a processor executes a software program to download the first known non-target organism (NTO) phosphorylation site sequence from a database.

In an embodiment, the processor is operatively linked to an electronic database of phosphorylation site sequence data.

In an embodiment, the plurality of non-target organism (NTO) phosphorylation site sequences are depleted of duplicate or redundant known NTO phosphorylation site sequences to provide a set of non-redundant phosphorylation site sequences and the set of non-redundant phosphorylation site sequences are used to query the dataset comprising a plurality of TO polypeptide sequences.

While methods herein have been described for a single known non-target organism phosphorylation site sequence, it will be understood that where the method is repeated for a plurality of known NTO phosphorylation site sequences, one or more steps of the method for creating database entries for each of the plurality of known NTO phosphorylation site sequences may be performed simultaneously. For example, the plurality of known non-target organism (NTO) phosphorylation site sequences downloaded from a database may be simultaneously entered as queries into a search program for identifying one matching target organism phosphorylation site sequence for each of the plurality of known non-target phosphorylation site sequences.

In an embodiment, the non-target organism (NTO) phosphorylation site sequence comprises sequences from one, two, three or more NTOs. In an embodiment, the sequences are from 4, 5, 6, 7, 8, 9 or 10 NTOs. In an embodiment, the NTO is selected from human, mouse, rat and bovine.

In an embodiment, the phosphorylation site sequence (e.g. NTO and/or TO) comprises at least 5 residues. In another embodiment, the phosphorylation site sequence (e.g. NTO and/or TO) comprises at least 6 residues. In another embodiment, the phosphorylation site sequence consists of 30 or fewer than 30 residues. In another embodiment, the number of phosphorylation site sequence residues is equal to or less than 20 residues in length. In an embodiment, the number of phosphorylation site sequence residues is at least or equal to 7, at least or equal to 8 residues, at least or equal to 9 residues, at least or equal to 10 residues, at least or equal to 11 residues, at least or equal to 12 residues, at least or equal to 13 residues, or at least or equal to 14 residues. In another embodiment, the phosphorylation site sequence is equal to or less than 18, 17, 16 or 15 residues. In an embodiment, the phosphorylation site sequence is at or equal to 21, 22, 23, 24, 25, 26, 27, 28, 29 or 30 residues.

In an embodiment, the NTO polypeptide sequence is/comprises full length protein sequences. In another embodiment NTO polypeptide sequences comprise at least 30%, 40%, 50%, 60%, 70%, 80% of the corresponding protein sequence and/or for example at least 30, at least 40, at least 50, at least 75, at least 100, at least 150, at least 200, at least 250, at least 300 or more residues.

In an embodiment, information pertaining to the first known NTO phosphorylation site sequence is retrieved when the sequence file is downloaded from the database. For example, the sequence file may contain the NTO phosphorylation site sequence accession number, the NTO phosphorylation site sequence, NTO phosphorylation site sequence description, NTO phosphorylation site sequence organism, NTO phosphorylation site sequence site, and NTO phosphorylation site sequence length. When the sequence file is downloaded from the PhosphoSitePlus data, the file may further contain the NTO phosphorylation site sequence low throughput references and/or the NTO phosphorylation site sequence high throughput references. One or more of these pieces of information may be included in the plurality of output values that are then displayed or included in the species-specific phosphorylation site database entry created according to the method.

The number of low-throughput references and high-throughput references are provided for example by the PhosphoSitePlus database for each known phosphorylation site.

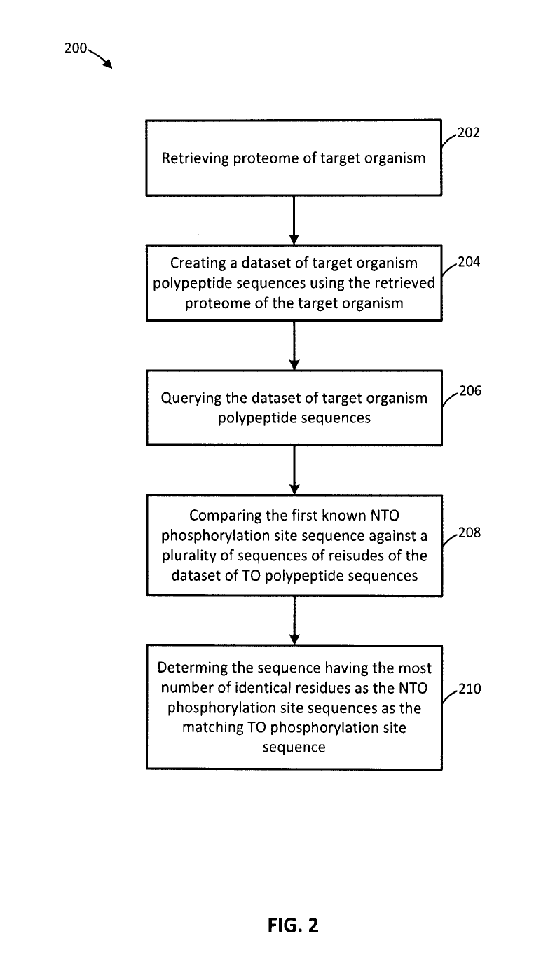

Referring now to FIG. 2, therein illustrated is a schematic diagram of a method 200 according to some exemplary embodiments for identifying a matching target organism (TO) phosphorylation site sequence for the first known NTO phosphorylation site sequence. For example, method 200 may be carried out at step 106 of method 100.

In an embodiment, identifying the matching TO phosphorylation site sequence and its cognate TO phosphorylation polypeptide sequence comprises, for example at step 206 of method 200, querying a dataset comprising a plurality of target organism (TO) polypeptide sequences with the known NTO phosphorylation site sequence (e.g. query phosphorylation site sequence) to identify a matching TO phosphorylation site sequence, and obtaining the accession number of the matching TO phosphorylation site sequence to thereby identify the cognate TO phosphorylation polypeptide sequence.

In an embodiment, the method of preparing a species-specific phosphorylation site database entry for a target organism comprises:

a) selecting a first known non-target organism (NTO) phosphorylation site sequence (Q) from a first NTO (Q_{O}), the known NTO phosphorylation site sequence comprising a length (Q_{L}) of at least 5 residues and less than 30 residues and/or 30 or fewer residues;

b) obtaining for the first known NTO phosphorylation site sequence, a first cognate known NTO phosphorylation polypeptide sequence (Q_{F}) and/or accession number (Q_{A}) corresponding to the known NTO phosphorylation site sequence, wherein the known NTO phosphorylation polypeptide sequence comprises the known NTO phosphorylation site sequence;

c) identifying a matching TO phosphorylation site sequence (H) for the known NTO phosphorylation site sequence, the matching TO phosphorylation site sequence comprising a length (H_{L}) of at least 5 residues and less than 30 residues and/or 30 or fewer residues;

d) obtaining for the matching TO phosphorylation site sequence an accession number (H_{A}) and/or cognate TO phosphorylation polypeptide sequence (H_{F});

e) identifying for the cognate known NTO phosphorylation polypeptide sequence (Q_{F}) (e.g. query polypeptide sequence) a matching TO phosphorylation polypeptide sequence for example by querying the dataset comprising the plurality of TO polypeptide sequences (T_{P});

f) determining a plurality of output values, one or more of the output values being indicative of a degree of matching between the TO phosphorylation site sequence and the NTO phosphorylation site sequence;

g) determining a similarity value between the first cognate NTO phosphorylation polypeptide sequence (Q_{F}) and the cognate TO phosphorylation polypeptide sequence (H_{F}), for example by determining if the matching TO phosphorylation polypeptide sequence and the cognate TO phosphorylation sequence are the same sequence and/or have the same accession number;

wherein the similarity value provides an indication of whether the first known NTO phosphorylation polypeptide sequence and the cognate TO phosphorylation polypeptide sequence are orthologues of each other.

For example, a similarity value is calculated between the cognate known NTO polypeptide sequence, and the cognate TO phosphorylation polypeptide sequence (corresponding to the TO phosphorylation site sequence identified for example by BLAST searching a NTO phosphorylation site sequence in the TO proteome).

In an embodiment, step c) comprises querying a dataset comprising a plurality of target organism (TO) polypeptide sequences (T_{P}) with the first known NTO phosphorylation site sequence (e.g. query phosphorylation site sequences) to identify a matching TO phosphorylation site sequence (H).

In an embodiment, step d) comprises querying the dataset comprising the plurality of TO polypeptide sequences with the matching TO phosphorylation site sequence to obtain an accession number (H_{A}) and/or cognate TO phosphorylation polypeptide sequence (H_{F}).

In an embodiment, the method further comprises populating a database with matching TO phosphorylation site sequences and/or related information optionally when known non-target polypeptide sequence (Q_{F}) and the cognate TO phosphorylation polypeptide (H_{F}) are orthologues e.g. reciprocal polypeptides.

In an embodiment, the database is populated with the matching TO phosphorylation site sequences and cognate TO sequence accession numbers when the similarity value is below a preselected threshold.

The phosphorylation site sequences of a TO that correspond to NTO phosphorylation site sequences can be selected for inclusion in an array, such as a kinome array. Accordingly, in an aspect, the disclosure provides a method of selecting sequences for preparing a species-specific phosphorylation site array for a target organism comprising:

a) selecting a first known non-target organism (NTO) phosphorylation site sequence of a first non-target organism, the first known NTO phosphorylation site sequence comprising at least 5 residues and less than 30 residues and/or 30 or fewer residues;

b) obtaining for the first known NTO phosphorylation site sequence a first cognate known NTO phosphorylation polypeptide sequence corresponding to the first known NTO phosphorylation site sequence, the cognate known NTO phosphorylation polypeptide sequence comprising the first known NTO phosphorylation site sequence;

c) identifying a matching target organism (TO) phosphorylation site sequence for the first known NTO phosphorylation site sequence;

d) obtaining for the matching TO phosphorylation site sequence a cognate TO phosphorylation polypeptide sequence corresponding to the matching TO phosphorylation site sequence, the cognate TO phosphorylation polypeptide sequence comprising the matching TO phosphorylation site sequence;

e) determining a plurality of output values, one or more of the output values being indicative of a degree of matching between the TO phosphorylation site sequence and the NTO phosphorylation site sequence;

f) determining a similarity value between the first known NTO phosphorylation polypeptide sequence and the cognate TO phosphorylation polypeptide sequence, wherein the similarity value provides an indication of whether the first known NTO phosphorylation polypeptide sequence and the cognate TO phosphorylation polypeptide sequence are orthologues of each other; and

g) selecting the matching TO phosphorylation site sequences determined to correspond to orthologue polypeptides for inclusion in the array.

In an embodiment, the matching TO phosphorylation site sequence is selected for the array when the similarity value is below a preselected threshold.

The dataset comprising the plurality of target organism (TO) polypeptide sequences (Tp) comprises for example, the TO proteome, optionally the full proteome or a subset thereof, e.g. a subproteome. A subproteome may be desired if for example the database and/or array is desired to be limited to a particular subset (e.g. immune system proteins). Alternatively, the desired subset can be selected subsequently, for example filtering a set of identified matching target organism phosphorylation site sequences for a desired subset such as immune system proteins.

In an embodiment, the dataset comprising the plurality of TO phosphorylation polypeptide sequences is prepared by first retrieving, for example at step 202 of method 200, a proteome of the target organism, for example from an available database of proteomes. The dataset of TO phosphorylation polypeptide sequences is then created, for example at step 204 of method 200, using the retrieved proteome of the target organism. It will be understood that the dataset of TO phosphorylation polypeptide sequences is a database of sequences that may be queried. For example, the dataset of TO phosphorylation sequences can be a BLAST database that is created using the makeblastdb program being run on the retrieved proteome of the target organism.

In an embodiment, the dataset is the TO proteome and is optionally downloaded. For example, a proteome of the target organism wherein the target organism is bovine can be downloaded from The International Protein Index (IPI) for example from (Citation for IPI: P J Kersey, J Duarte, A Williams, Y Karavidopoulou, E Birney, and R Apweiler. The International Protein Index: an integrated database for proteomics experiments. Proteomics, 4(7):1985-8, 2004). Integr8 can also be used (citation for Integr8; P Kersey, L Bower, L Morris, A Home, R Petryszak, C Kanz, A Kanapin, U Das, K Michoud, I Phan, A Gattiker, T Kulikova, N Faruque, K Dug-gan, P Mclaren, B Reimholz, L Duret, S Penel, I Reuter, and R Apweiler. Integr8 and Genome Reviews: integrated views of complete genomes and proteomes. Nucleic Acids Res, 33(Database issue):D297-302, 2005).

In an embodiment, a processor executes a software program to retrieve the proteome of the target organism from an electronic database of polypeptide sequence data.

In an embodiment, the processor is operatively linked to an electronic database of polypeptide sequence data.

In an embodiment, the dataset comprising the plurality of TO phosphorylation site sequences is created by first downloading the TO proteome from one of the sources listed above and then created by running the makeblastdb program on the TO proteome in order to create a BLAST database comprising a plurality of TO phosphorylation polypeptide sequences. The created BLAST database can then be queried using other functions and programs, such as blastp, in order to identify a matching TO phosphorylation site sequence for the first known NTO phosphorylation site sequence.

In an embodiment, one or more data sets are obtained in nucleotide format and translated in one or all reading frames to provide a database containing polypeptide sequences. For example, if nucleotide TO sequence data is obtained, for example as a collection of cDNAs, the cDNA is translated to polypeptide sequence—if a start codon is unknown, the cDNA sequence can be translated in all reading frames.

Alternatively in another embodiment, nucleotide databases can be employed where the query sequences are for example nucleotide sequences corresponding to polypeptide sequences.

In an embodiment, the sequences (e.g. the NTO phosphorylation site sequences, the NTP phosphorylation polypeptide sequences, the TO polypeptide sequences, the TO phosphorylation site sequences and/or the TO phosphorylation polypeptide sequences or any other sequences described herein) are in FASTA format. In another embodiment, the sequences are in raw, GCG, GenPept, XML, EMBL, Swiss-PROT, PIR and/or PDB formats. Other formats can also be used.

The NTO phosphorylation site sequence is compared, for example at step 208 of method 200, against each of TO polypeptide site sequences of the dataset in order to identify a matching TO polypeptide site sequence. The known NTO phosphorylation sites are for example compared against the full proteins in the target proteome (using, for example, a local alignment such as BLAST). As another example, the comparing comprises comparing, for example at step 210 of method 200, the alignment of residues of the NTO phosphorylation site sequence against the residues of each of the plurality of TO polypeptide site sequences to find the number of identical residues between the NTO phosphorylation site sequence and each of the TO phosphorylation site sequences. The TO phosphorylation site sequence that contains the best match in terms of number identical residues is identified as the matching TO phosphorylation site sequence.

In an embodiment, the matching TO phosphorylation site sequences and/or the matching TO phosphorylation polypeptide sequences are identified using a blastp search. For example, the nonredundant phosphorylation site sequences optionally in FASTA format are used as queries in a BLAST search for example the stand-alone version of blastp (ftp://ftp.ncbi.nlm.nih.gov/blast/executables/blast+/LATEST), with the dataset of TO phosphorylation polypeptide sequences that is queried. The blastp search may be performed using the -ungpapped option in order to produce an ungapped alignment of residues.

After identifying a matching TO phosphorylation site sequence, a cognate TO phosphorylation polypeptide sequence corresponding to the matching TO phosphorylation site sequence is obtained. The corresponding cognate TO phosphorylation polypeptide sequence comprises the matching TO phosphorylation site sequence.

In an embodiment, the querying of the dataset of TO phosphorylation polypeptide sequences with the known NTO phosphorylation site sequence to identify a matching TO phosphorylation site sequence generates a query output that comprises information about the matching TO phosphorylation site sequence including, for example, matching TO phosphorylation site sequence accession number, matching TO phosphorylation site sequence description, number of sequence identities in the residue alignment between the known NTO phosphorylation site sequence and the matching TO phosphorylation site, matching TO phosphorylation site sequence, and the matching TO phosphorylation site sequence start position relative to the cognate TO phosphorylation polypeptide sequence. One or more of these pieces of information may be included in the plurality of output values that are then displayed or included in the species-specific phosphorylation site database entry created according to the method.

In an embodiment, the query output is parsed to extract information about the matching TO phosphorylation site sequence. Where the query is performed as a blast search using blastp, the query output may be parsed using BioPerl module SearchIO which parses the text output from BLAST, allowing the relevant information for the query output to be easily extracted in an automated fashion.

The matching polypeptide or matching phosphorylation site sequence can for example be the best match e.g. the one with the smallest E-value. For example, no match is identified if the smallest E-value is larger than 10, which is the default “Expect threshold” used by BLAST. In an embodiment, matches with E-values greater than the expect threshold are not reported at all. In another embodiment, more than one “match” is selected e.g. the best two, three, four etc. matches are selected. In an embodiment, each of the selected matches are compared to the cognate TO phosphorylation sequence and the match with for example the same accession number is selected.

In an embodiment, the number of sequence differences (e.g. number of non-exact residue matches) between the NTO phosphorylation site sequence (e.g. the entire query sequence and not just the portion of the query sequence that matched) and the matching TO phosphorylation site sequence (e.g. best hit sequence and only the portion that matched) is then calculated. The number of sequence differences is indicative of a degree of matching between the TO phosphorylation site sequence and the NTO phosphorylation site sequence. In an embodiment, the Levenshtein edit distance is calculated. In an embodiment, the number of sequence differences is up to 80%, 70%, 60%, 50%, 40%, 35%, 30%, 25%, 20%, 15% or 10%. In an embodiment, the matching sequence has 0 sequence differences. For example, where the input sequence is 8 amino acid residues, the matching sequence may have 6 or less, 5 or less, 4 or less, 3 or less, 2 or less, 1 or no sequence differences (e.g. 0 sequence differences) and where the input sequence is 15 amino acid residues, the matching sequence may have 12 or less, 11 or less, 10 or less, 9 or less, 8 or less, 7 or less, 6 or less, 5 or less, 4 or less, 3 or less, 2 or less, 1 or no sequence differences (0 sequence differences). Sequences having more than for example 60% different residues are not considered matches. The number of sequence differences may be calculated as the difference between the NTO phosphorylation site sequence length and the number of sequence identities described above. The number of sequence differences may be included in the plurality of output values that are displayed in the species-specific phosphorylation site database entry created according to the method.

In an embodiment, the number of non-conservative sequence differences between the NTO phosphorylation site sequence (e.g. the entire query sequence and not just the portion of the query sequence that matched) and the matching TO phosphorylation site sequence (e.g. best hit sequence and only the portion that matched) is then calculated. The number of sequence differences is indicative of a degree of matching between the TO phosphorylation site sequence and the NTO phosphorylation site sequence. In an embodiment, the number of non-conservative sequence differences is up to 90%, 80%, 70%, 60%, 50%, 40%, 35%, 30%, 25%, 20%, 15% or 10%. For example, where the input sequence is 8 amino acid residues, the matching sequence may have 8 or less, 7 or less, 6 or less, 5 or less, 4 or less, 3 or less, 2 or less, 1 or no non-conservative sequence differences and where the input sequence is 15 amino acid residues, the matching sequence may have 6 or less, 5 or less, 4 or less, 3 or less, 2 or less, 1 or no non-conservative sequence differences. Sequences having more than for example 60% different residues are not considered non-conservative matches. The number of sequence differences may be calculated as the difference between the NTO phosphorylation site sequence length and the sum of the number of sequence identities mentioned and the number of non-conservative substitutions. The number of non-conservative sequence differences may be included in the plurality of output values that are displayed in the species-specific phosphorylation site database entry created according to the method.

In an embodiment, the method comprises comparing the full protein sequence of for example the mature (or other species) TO phosphorylation sequence polypeptide with the full protein of for example the mature (or other species) NTO phosphorylation sequence polypeptide.

In an embodiment, the identifying of the matching TO phosphorylation site sequence further comprises determining the hit site of the TO phosphorylation site sequence. This hit site corresponds to the site of the phosphorylated residue within the cognate TO phosphorylation polypeptide sequence. In an embodiment, where the length of the known phosphorylation site sequence is for example equal to 15, and the phosphorylation site residue in the known phosphorylation site sequence is at position 8, the hit site can be calculated according to the expression Hs−Qs+8 where Hs is the start position of the hit in the matching TO phosphorylation site sequence and Qs is the start position in the known NTO phosphorylation site sequence for example as reported by local alignment (e.g. BLAST). A person skilled in the art would understand that if the phosphorylated residue in the known phosphorylation site is at another position, e.g. position 9 of the known phosphorylation site sequence of length 17, the hit site can be calculated according to the expression Hs−Qs+9. The hit site may be included in the plurality of output values that are displayed in the species-specific phosphorylation site database entry created according to the method.

In an embodiment, the identifying of the matching TO phosphorylation site sequence further comprises calculating the n-mer optionally 9-mer, sequence differences and the n-mer, optionally 9-mer, non-conservative sequence differences. For example, a 9-mer or 9-amino-acid-long substring of a 15 amino acid NTO phosphorylated site sequence (Q⁹) where the phosphorylated residue is its central residue, is identified by locating the phosphorylated residue of the NTO phosphorylated site sequence and the 4 indices (residues) on either side of the phosphorylated residue (e.g. residues 4 to 12 inclusive). Similarly, a 9-amino-acid-long substring of the TO phosphorylated site sequence (e.g. H⁹, where H_{L} is at least 9 residues long) where the phosphorylated residue is at its centre, is identified, by locating the phosphorylated residue of the TO phosphorylated site sequence and for example by taking the substring between indices (5−Q₅) and (13−Q_{S}) inclusive. A person skilled in the art would recognize that if Q_{L}, the length of the known NTO phosphorylation site sequence, is not 15, the indices will vary accordingly. Depending on the selected n-mer, selected to query lengths and identified hit lengths, a person skilled in the art would be able to modify the above equations accordingly.

In an embodiment, the 9-mer sequence differences is calculated as the number of sequence differences between the TO phosphorylated site sequence. 9-amino-acid long substring (H⁹) and the query 9-amino acid long substring (Q⁹). In an embodiment, the 9-mer non-conservative sequence is calculated as the number of non-conservative sequence differences between the TO phosphorylated site sequence 9-amino-acid long substring and the query 9-amino acid long substring.

As described, the NTO phosphorylation site sequences are at least 5 amino acid residues and up to for example 30 amino acid residues in length. Due to the short length of the query sequences, the cognate TO phosphorylation polypeptide sequence corresponding to the matching TO phosphorylation site sequence may not be orthologous to the first known NTO phosphorylation polypeptide sequence.

Orthology can be assessed for example by identifying reciprocal blast hits as outlined herein (e.g. below and under description of DAPPLE and Detailed description of DAPPLE methodology). Orthology can also be assessed by selecting a threshold and sequences sharing an E-value below the threshold are likely to be orthologues (e.g. ABC).

In an embodiment, reciprocal blast hits are identified and the following further comparisons are made. A comparison is made between the first known NTO phosphorylation polypeptide sequence to each of the TO phosphorylation polypeptide sequences of the dataset of TO phosphorylation polypeptide sequences, to generate a plurality of TO dataset similarity values. A best TO dataset similarity value is identified, E¹_{B}. The comparison step also includes in an embodiment identifying a first TO dataset similarity value of the match between first known NTO phosphorylation polypeptide sequence (Q_{F}) and the cognate TO phosphorylation polypeptide sequence (H_{F}) from the plurality of TO dataset similarity values, E¹_{F}. The similarity rank (e.g. E-value rank) (S) of the match between the first known NTO phosphorylation polypeptide sequence and the cognate TO phosphorylation polypeptide is further determined. The comparison may be performed, for example as a blastp search using the first cognate known NTO phosphorylation polypeptide sequence as the query and the dataset of TO phosphorylation polypeptides as the queried database, in which case the TO dataset similarity values are a plurality of E-values, wherein the smallest E-value is identified as the best TO dataset similarity value.

Another comparison is made between the cognate TO phosphorylation polypeptide sequence to each of the NTO phosphorylation polypeptide sequences of the dataset of NTO phosphorylation polypeptide sequences, to generate a plurality of NTO dataset similarity values. A best NTO dataset similarity value is identified, E²_{B}. In an embodiment, the method further comprises identifying a first NTO dataset similarity value of the match between the first known NTO phosphorylation polypeptide sequence (Q_{F}) and the cognate TO phosphorylation polypeptide sequence (H_{F}) from the plurality of NTO dataset similarity values.

In some embodiments, the non-target proteome is downloaded from The International Protein Index (IPI) for example from ftp://ftp.ebi.ac.uk/pub/databases/IPI/current/ipi.BOVIN.fasta.gz (Citation for IPI: P J Kersey, J Duarte, A Williams, Y Karavidopoulou, E Birney, and R Apweiler. The International Protein Index: an integrated database for proteomics experiments. Proteomics, 4(7):1985-8, 2004). Integr8 can also be used (citation for Integr8; P Kersey, L Bower, L Morris, A Horne, R Petryszak, C Kanz, A Kanapin, U Das, K Michoud, I Phan, A Gattiker, T Kulikova, N Faruque, K Dug-gan, P Mclaren, B Reimholz, L Duret, S Penel, I Reuter, and R Apweiler. Integr8 and Genome Reviews: integrated views of complete genomes and proteomes. Nucleic Acids Res, 33(Database issue):D297-302, 2005). The makeblastdb program is then run on the NTO proteome in order to create a BLAST database comprising a plurality of NTO phosphorylation polypeptide sequences. The BLAST database forms the dataset of NTO phosphorylated polypeptide sequences. In this case, the second comparison may be performed as a blastp search using the cognate TO phosphorylation polypeptide sequence as the query and the dataset of NTO phosphorylation polypeptides as the queried database, in which case, the NTO dataset similarity values are a plurality of E-values, wherein the smallest E-value is identified as the best NTO dataset similarity value.

The best TO dataset similarity value E¹_{B} is then compared against the first TO dataset similarity value E¹_{F} and the best NTO dataset similarity E²_{B} value is compared against the first NTO dataset similarity value E²_{F}, wherein if the first TO dataset similarity value E¹_{F} equals the best TO dataset similarity value E¹_{S} and the first NTO dataset similarity value E²_{F} equals the best NTO dataset similarity value E²_{B}, the cognate TO phosphorylation polypeptide sequence is determined to be an orthologue of, or reciprocal blast hit of, the first known NTO phosphorylation polypeptide sequence. An indication of whether the TO phosphorylation polypeptide sequence is an orthologue of the first known NTO phosphorylation polypeptide sequence is included in the plurality of output values. In some embodiments, the plurality of output values may further include the first TO and/or NTO dataset similarity value. In some embodiments, the plurality of output values may further include the hit polypeptide sequence rank, which is determined as the rank of the first TO and NTO dataset similarity values amongst the plurality of TO and NTO dataset similarity values.

An example of the above steps for performing the reciprocal blast hit comparison is outlined in steps 332-340 under the heading Detailed description of DAPPLE methodology.

In an embodiment, the reciprocal blast hit comparison comprises the following:

a) run blastp using Q_{F} as the query and DT_{P} as the database. Determine the E-value E1_{B} of the best BLAST hit, and also the E-value E¹_{F} of the match between Q_{F} and H_{F}. Also, let S be the E-value rank of the E¹_{F}. In other words, if E¹_{F} is the n^{th} smallest E-value, then S=n.

b) run blastp using H_{F} as the query and DQo_{P} as the database. Determine the E-value E²_{B} of the best BLAST hit, and also the E-value E²_{F} of the match between Q_{F} and H_{F}.

c) let R=“yes” if Q_{F} and H_{F} are reciprocal BLAST hits, and “no” otherwise; if E¹_{B}=E¹_{F} and E²_{B}=E²_{F}, then R=“yes”; otherwise, R=“no”.

The series of comparisons can also be understood for example according to the following:

Assume, to start, that the NTO phosphorylation polypeptide sequence and the cognate TO phosphorylation polypeptide sequence are reciprocal BLAST hits. This assumption is maintained until proven otherwise. Blastp is executed using the NTO phosphorylation polypeptide sequence as the query and the TO proteome as the database. If the E-value against the cognate TO phosphorylation polypeptide sequence is not equal to the best (smallest) E-value of all the hits against the TO proteome, then the two proteins are not reciprocal BLAST hits. Then, blastp is executed using the TO phosphorylation polypeptide sequence as the query and the NTO proteome as the database. If the E-value against the NTO phosphorylation polypeptide sequence is not equal to the best (smallest) E-value, then the two proteins are not reciprocal BLAST hits.

The comparisons may be performed, for example, as a blastp search, in which case the first similarity value is an E-value, wherein similar sequences will have a small E-value and dissimilar sequences will have a large E-value, of the match recorded. If the similarity value is large for example if the E-value is large, then the two proteins may not be orthologues. Although percent identity can be used, E-value is preferred for determining orthology as it takes into account sequence length, database size, etc.

In an embodiment, the similarity value comprises an E-value. In another embodiment, the E-value is selected at less than 10⁻², 10⁻³, 10⁻⁴, 10⁻⁵, between 10⁻² and 10⁻⁵ or any number in between.

The comparison may be performed, for example, as a blastp search using the first known NTO phosphorylation polypeptide sequence as a query, in which case the first similarity value is an E-value, wherein similar sequences will have a small E-value and dissimilar sequences will have a large E-value, of the match recorded. If the similarity value is large for example if the E-value is large, then the two proteins may not be orthologues. Although percent identity can be used, E-value is preferred for determining orthology as it takes into account sequence length, database size, etc.

In an embodiment, the similarity value comprises an E-value. In another embodiment, the E-value is selected at less than 10⁻², 10⁻³, 10⁻⁴, or 10⁻⁵, or between 10⁻² and 10⁻⁵ or any number in between.

In an embodiment, the plurality of output values is displayed.

In an embodiment, the plurality of output values is outputted to form an entry for the species-specific phosphorylation site database. The plurality of output values may be outputted electronically to allow easy importing into a spreadsheet program. For example, the output may be in tab-delimited plain text format, comma-delimited plaint text format, or any other delimited format for easy importing.

Since the method may be repeated for a large number of NTO phosphorylated site sequences, for example thousands of sequences, output values for a large number of database entries may be prepared. In an embodiment, the method further includes a method of filtering the table so that one can intelligently choose which peptides for example to include on the kinome array. For example, the user may wish to view only entries where the number of low-throughput references is greater than two, or to eliminate entries where the similarity value is greater or lesser than a certain threshold.

In an embodiment, the method is computer implemented. In an embodiment the method is carried using the “DAPPLE” program described herein which uses for example, a reciprocal BLAST hit (RBH) component to ascertain orthology or the ABC program described under the heading ABC, which specifies an E-value threshold for determining orthology. The DAPPLE program also allows selection of an E-value threshold. In another embodiment, a computerized system implements the method described above. In an embodiment, the computerized system carries out the “DAPPLE” program for example as more particularly described under the headings DAPPLE and Detailed description of DAPPLE methodology or the ABC program described below under the heading ABC.

In an embodiment, the BLAST searches can be parallelized and the computer method (e.g. DAPPLE) can be run on a workstation cluster or computer grid to reduce its computational time.

In another embodiment, a non-first match is used, especially if the full protein corresponding to one of these matches is orthologous to the full protein corresponding to the query.

In another embodiment, the substitution matrix is based on the evolutionary relatedness between the target organism and the organism corresponding to a given known phosphorylation site.

A further aspect comprises a non-transitory computer-readable storage medium comprising a plurality of instructions, wherein the instructions, when executed, cause a processor to perform the following:

a) querying a dataset comprising a plurality of target organism (TO) polypeptide sequences with a selected plurality of known NTO phosphorylation site sequences (query phosphorylation site sequences) to identify for each of the plurality of NTO phosphorylation site sequences a matching TO phosphorylation site sequence;

b) obtaining for each of the matching TO phosphorylation site sequences a cognate TO phosphorylation polypeptide sequence corresponding to the matching TO phosphorylation site sequence, the cognate TO phosphorylation polypeptide sequence comprising the matching TO phosphorylation site sequence;

c) determining a plurality of output values, one or more of the output values being indicative of a degree of matching between the TO phosphorylation site sequence and the NTO phosphorylation site sequence; and

d) determining a similarity value between the first known NTO phosphorylation polypeptide sequence and the cognate TO phosphorylation polypeptide sequence, wherein the similarity value provides an indication of whether the first known NTO phosphorylation polypeptide sequence and the cognate TO phosphorylation polypeptide sequence are orthologues of each other.

In an embodiment, the instructions are further for performing the step of displaying the matching TO phosphorylation site sequences and/or cognate TO sequence accession numbers when the similarity value is below a preselected threshold.

In an embodiment, the instructions are further for performing the step of displaying one or more of these pieces of information of the plurality of output values. In an embodiment, the instructions are further for performing the steps of creating a species-specific phosphorylation site database entry.

In an embodiment, the instructions stored on the non-transitory computer-readable medium are further for performing the step of carrying out the steps of any one or more of the methods described herein.

A further aspect comprises a system for preparing one or more species-specific phosphorylation site database entries for a target organism, the system comprising:

a) a memory for storing a plurality of instructions; and

b) a processor coupled to the memory for:
i) obtaining for the first known non-target organism (NTO) phosphorylation site sequence of a first non-target organism, the first known NTO phosphorylation site sequence comprising at least 5 residues and less than 30 residues and/or 30 or fewer residues, a first cognate known NTO phosphorylation polypeptide sequence corresponding to the first known NTO phosphorylation site sequence, the cognate known NTO phosphorylation polypeptide sequence comprising the first known NTO phosphorylation site sequence;
ii) identifying a matching target organism (TO) phosphorylation site sequence for the first known NTO phosphorylation site sequence;
iii) obtaining for the matching TO phosphorylation site sequence a cognate TO phosphorylation polypeptide sequence corresponding to the matching TO phosphorylation site sequence, the cognate TO phosphorylation polypeptide sequence comprising the matching TO phosphorylation site sequence;
iv) determining a plurality of output values, one or more of the output values being indicative of a degree of matching between the TO phosphorylation site sequence and the NTO phosphorylation site sequence; and
v) determining a similarity value between the first known NTO phosphorylation polypeptide sequence and the cognate TO phosphorylation polypeptide sequence, wherein the similarity value provides an indication of whether the first known NTO phosphorylation polypeptide sequence and the cognate TO phosphorylation polypeptide sequence are orthologues of each other.

In an embodiment, the similarity value is an E-value and the preselected threshold is 10⁻³.

In an embodiment, the program comprises the DAPPLE scripts described below under the heading Detailed description of DAPPLE methodology.

Another aspect includes a computerized control system for controlling and receiving data, the computerized control system comprising at least one processor and memory configured to provide:

a) a control module for:
i) receiving one or more NTO phosphorylation site sequence datasets comprising a plurality of NTO phosphorylation site sequences, and one or more TO polypeptide sequence datasets comprising a plurality of TO polypeptide sequences; and
ii) receiving a selected similarity value threshold;

b) an analysis module for:
i) querying a dataset comprising a plurality of target organism (TO) polypeptide sequences with a selected plurality of known NTO phosphorylation site sequences (query phosphorylation site sequences) to identify a matching TO phosphorylation site sequence and cognate TO phosphorylation polypeptide sequence and/or sequence accession number, for each of one or more of the plurality of known NTO phosphorylation site sequences;
ii) querying the dataset comprising the plurality of TO polypeptide sequences with a cognate known NTO phosphorylation polypeptide sequence (query polypeptide sequences) corresponding to each known NTO phosphorylation site sequence to identify for each cognate known NTO phosphorylation polypeptide sequence, a matching TO phosphorylation polypeptide sequence;
iii) calculating, for each of the plurality of known NTO phosphorylation site sequences, a similarity value between the cognate TO phosphorylation polypeptide sequence and the matching TO phosphorylation polypeptide sequence; and

c) a display module for displaying the matching TO phosphorylation site sequences and/or cognate TO sequence accession numbers when the similarity value is below a preselected threshold.

### ABC Method

Using the cow as a test species, a protocol for designing kinome microarrays for species with few known phosphorylation sites was recently proposed (Jalal et al., 2009). Taking advantage of sequence homology between human proteins and bovine proteins, this study used known human phosphorylation sites as BLAST (Altschul et al., 1997) queries in order to identify probable bovine sites. If a given query's best match in the bovine proteome had few sequence differences relative to the query, it was a candidate for inclusion on a bovine-specific kinome microarray. While useful, several aspects of this protocol could be improved.

First, the manual nature of the protocol makes it time-consuming and tedious to perform, and also limits the amount of known phosphorylation data that can be used. Second, the protocol uses only known phosphorylation sites from human. This is problematic because it is possible, for instance, that a given bovine phosphorylation site might be homologous to a known rat phosphorylation site, but not to any known human site. By using only known phosphorylation sites from human, this bovine site would be missed. Third, the method used by the protocol to identify possible non-orthologous proteins (comparing the annotations of those proteins) has several drawbacks, including the subjective nature of comparing annotations, the difficulty of automating these comparisons, and the fact that protein annotations are often inaccurate or incomplete. Fourth, the protocol described in Jalal et al. (2009) has no facility for choosing which peptides should be included on the array once the BLAST searches have been performed.

ABC is a collection of Perl scripts that addresses these concerns, ultimately allowing the user to easily, quickly, and accurately identify potential phosphorylation sites in an organism of interest.

To test ABC, it was used to identify phosphorylation sites in the cow (Bos taurus), just as was done in Jalal et al. (2009). The PhosphoSitePlus database was downloaded on Feb. 14, 2011, and contained 97679 known phosphorylation sites (83860 of them unique). The International Protein Index (IPI) (Kersey et al., 2004) bovine proteome was downloaded from ftp://ftp.ebi.ac.uk/pub/databases/IPI/current/ipi.BOVIN.fasta.gz on Dec. 20, 2010 in FASTA format and contained 29384 protein sequences. These two files were then used as input to ABC. For comparison purposes, the output table produced by ABC was used to generate summary data similar to that in Table 1 of Jalal et al. (2009). Note that the methodology employed by ABC is not identical to that employed by Jalal et al., so the results that it produces are not expected to be exactly the same. Table 1 compares the summary results given by Jalal and coauthors in Table 1 of their paper with the results produced by ABC.

As can be seen, the percentages of known phosphorylation sites that had a given number of sequence differences with their best bovine BLAST match were similar between the two approaches. The percentage of queries for which no homology was found in the bovine proteome was also similar, despite the different approaches used for detecting non-homology.

As kinome microarrays become a more popular tool for studying cellular signaling, the ability to design kinome microarrays suitable for studying different species will become increasingly important.

ABC improves upon an already-successful method for designing kinome microarrays. Compared to the previous protocol, it is far less time-consuming and tedious, yet is able to make use of 100 times more information. Through its use of all known phosphorylation sites in the PhosphoSitePlus database, rather than just those from human, ABC is more robust and thorough. Finally, the program greatly improves the ability to identify non-orthologous matches. As such, ABC should prove to be a useful tool for designing species-specific kinome microarrays.

TABLE 1
Comparison of the results of Jalal et al. (2009) with those
of ABC when finding potential phosphorylation sites in the
bovine proteome. The first column indicates the number
of sequence differences between a known phosphorylation
site from the PhosphoSitePlus database, and its best match
in the bovine proteome. The second column indicates the
percentage of known phosphorylation sites with the indicated
number of sequence differences in Jalal et al. (2009), while the third
column has the same meaning, except for data produced by ABC.

| Sequence differences | % (Jalal et al.) | % (ABC) |
|---|---|---|
| 0 | 50% | 33.0% |
| 1 | 13% | 17.3% |
| 2 | 7% | 11.3% |
| 3 | 4% | 7.9% |
| 4 | 1.5% | 5.6% |
| 5 | 0.4% | 4.0% |
| 6+ | 0.6% | 3.1% |
| 0 to 15 (no homology)* | 22% | 17.8% |

*In the paper by Jalal and co-authors, this row indicates known phosphorylation sites for which there was either no match in the bovine proteome, or the annotation of the match did not match the annotation of the query. For ABC, this row indicates phosphorylation sites for which there was either no match in the bovine proteome, or the E-value between the two full proteins (see Methods) was greater than 10⁻³.

### Materials and Methods

ABC requires two input files: the proteome of the target organism (for which the user wants to design the kinome microarray) in FASTA format, and the phosphorylation site data from PhosphoSitePlus, which can be obtained from www.phosphosite.org/downloads/Phosphorylation site dataset.gz. As the PhosphoSitePlus data file contains entries with identical sequences (from different organisms), duplicate sequences are first removed. A FASTA file containing the nonredundant phosphorylation sites is then created, and the sequences in this file are used as queries to the stand-alone version of blastp (ftp://ftp.ncbi.nlm.nih.gov/blast/executables/blast+/LATEST), with the target organism's proteome as the database. Unlike in Jalal et al., the queries are not limited to those from human. The output from blastp is then parsed using the BioPerl (Stajich et al., 2002) module SearchIO, and the accession number and sequence of the best match, if any, for each query are saved. The number of sequence differences—or, more formally, the Levenshtein edit distance—between the entire query sequence (not just the portion of the query sequence that matched) and the best hit sequence (only the portion that matched) is then calculated.

Due to the short length of the query sequences (between eight and fifteen amino acids), the full protein corresponding to the best match may not be orthologous to the full protein corresponding to the query sequence. In Jalal et al. (2009), this problem was addressed by manually comparing the annotations of the proteins corresponding to the query and the match. However, this approach suffers from the drawbacks described in the introduction; thus, ABC instead uses the full protein corresponding to each known phosphorylation site (i.e. each of the original queries) as a blastp query against the target proteome. The match against the same accession number as was matched by the corresponding phosphorylation site is then identified, and the E-value of this match recorded. If this E-value is large, then the two proteins may not be orthologues. The output of ABC is a table in which each row represents the result of a BLAST search using, as a query, one of the phosphorylation sites in the PhosphoSitePlus data file. The table is in a tab-delimited plain text format that can easily be imported into a spreadsheet program. This table contains several columns, including: query accession (the accession number of the protein corresponding to the known phosphorylation site), query description (a description of that protein), query organism (the organism corresponding to that protein), query sequence (the amino acid sequence of the known phosphorylation site), hit accession (the accession number of the best match in the target proteome), hit sequence (the amino acid sequence of this match), sequence differences (the number of sequence differences between the entire query sequence and the portion of the hit protein that matched), protein E-value (the E-value between the entire protein corresponding to the query accession, and the entire protein corresponding to the hit accession), low-throughput references (the number of low-throughput references corresponding to this phosphorylation site), and high-throughput references. The rows are listed in increasing order of sequence differences.

Since the output table will contain thousands of possible phosphorylation sites, the user needs some method of filtering the table so that he or she can intelligently choose which peptides to include on the array. For example, the user may wish to view only rows where the number of low-throughput references is greater than two, or to eliminate rows where the E-value is greater than a certain threshold. ABC contains a number of scripts allowing the output table to be filtered in these and other ways, further aiding the user in designing species-specific kinome microarrays.

### Dapple

DAPPLE (Design Array for PhosPhoryLation Experiments) is a collection of Perl scripts that addresses the concerns listed for example in the description of ABC, ultimately allowing the user to easily, quickly, and accurately identify potential phosphorylation sites in an organism of interest.

### Methods

DAPPLE requires several input files: the proteome of the target organism (for which the user wants to design a kinome microarray) in FASTA format; the proteomes of the organisms represented in the database of phosphorylation sites, also in FASTA format; and the phosphorylation site data. If a particular organism represented in the phosphorylation site data does not have a proteome available, then the known phosphorylation sites from that organism can still be used; however, DAPPLE will be unable to output information for the “RBH?” column of the output table (see below). The phosphorylation site data could be obtained from a number of sources, including the PhosphoSitePlus database (Hornbeck et al., 2004), Phospho.ELM (DieIla et al., 2004, 2008), or the literature. This study used data from PhosphoSitePlus, which can be obtained from www.phosphosite.org/downloads/Phosphorylation site dataset.gz. As the PhosphoSitePlus data file contains entries with identical sequences (from different organisms), duplicate sequences are first removed. The sequences of the non-redundant phosphorylation sites are used as queries to the standalone version of blastp (ftp://ftp.ncbi.nlm.nih.gov/blast/executables/blast+/LATEST), with the target organism's proteome as the database. Unlike in Jalal et al. (2009), the queries are not limited to those from human. The output from blastp is then parsed using the BioPerl (Stajich et al., 2002) module SearchIO, and the accession number and sequence of the best match, if any, for each query are saved. If there are multiple matches with the same E-value as the best match, then only the first result returned by BLAST is used. Additional information about the match is then saved or computed, and ultimately presented in the DAPPLE output table (described below).

Due to the short length of the query sequences (between eight and fifteen amino acids), the full protein corresponding to the best match may not be orthologous to the full protein corresponding to the query sequence. In Jalal et al. (2009), this problem was addressed by manually comparing the annotations of the proteins corresponding to the query and the match. However, this approach suffers from the drawbacks described in the introduction; thus, DAPPLE uses the well-established reciprocal BLAST hits (RBH) method to ascertain orthology (Moreno-Hagelsieb and Latimer, 2008). For a given known phosphorylation site X from organism A with best match Y in organism B (the target organism), let X′ be the full protein corresponding to X, and Y′ be the full protein corresponding to Y. DAPPLE will declare X′ and Y′ as orthologues if and only if Y′ is the best match when X′ is used as a query sequence and the proteome of organism B is used as the database, and X′ is the best match when Y′ is used as a query sequence and the proteome of organism A is used as the database. In this case, “the best match” is defined as any protein that has the smallest E-value. For instance, if X′ is not the first result returned by BLAST when Y′ is used as a query sequence and the proteome of organism A is used as the database, then X′ and Y′ can still be declared as orthologues if the E-value of the match against X′ is equal to that of the first result returned by BLAST.

The output of DAPPLE is a table in which each row represents the result of a BLAST search using, as a query, one of the known phosphorylation sites in the PhosphoSitePlus data file. The table is in a tab-delimited plain text format that can easily be subsequently manipulated. This table contains many columns. The following list describes each column, with X, Y, X′, and Y′ having the same meaning as above.
Query accession—the accession number of X′.
Query description—a description of X′.
Query organism—the organism that encodes X′.
Query sequence—the amino acid sequence of X.
Query site—the phosphorylated residue in X′; e.g. Y482.
Hit site—the residue in Y′ that corresponds to the query site.
Hit accession—the accession number of Y′.
Hit description—a description of
Hit sequence—the amino acid sequence of Y.
Sequence differences—the number of sequence differences between the entirety of X (not just the portion that matched in the BLAST local alignment) and Y. For instance, if X=ABCDEFGH and Y=CDEFG, then the number of sequence differences would be 3.
Non-conservative sequence differences—as above, except counting only the number of non-conservative sequence differences (those with a score less than or equal to zero in the BLOSUM62 matrix).
9-mer sequence differences—the number of sequence differences between the nine-residue region centred at the phosphorylated residue of X, and the nine-residue region centred at the corresponding residue in Y.
9-mer non-conservative sequence differences—as above, except counting only the number of non-conservative sequence differences.
Hit protein rank—This column will be 1 if the E-value between X′ and Y′ when a blastp search is performed using X′ as the query and the target proteome as the database is equal to the smallest E-value returned by this search, even if Y′ is not the first result returned. Otherwise, it will be the number corresponding to the order in which Y′ is returned by BLAST. For instance, if the best hit has an E-value of 10⁻³² and Y′ is the fifth result returned and has an associated E-value of 10⁻²⁴, then this column will be 5. Hit protein E-value—the E-value of the match between X′ and Y′ when X′ is used as the query and the target organism is used as the database.
RBH?—either “yes” or “no”, depending on whether X′ and Y′ are reciprocal BLAST hits.
Low-throughput references—the number of references reporting the use of low-throughput biological techniques to study X.
High-throughput references—the number of references reporting the use of high-throughput biological techniques to study X.
   The rows are listed in increasing order of sequence differences.
   Since the output table will contain thousands of possible phosphorylation sites, the user needs some method of filtering the table so that he or she can intelligently choose which peptides to include on the array. For example, the user may wish to view only rows where the number of low-throughput references is greater than two, or to eliminate rows where the “RBH?” column is “no”. DAPPLE's documentation describes a number of UNIX commands that can be used to filter the output table in these and other ways, further aiding the user in designing species-specific kinome microarrays.

To test DAPPLE, phosphorylation sites in the cow (Bos taurus) were identified, just as was done in Jalal et al. (2009). The files described below, all of which were downloaded on Jun. 7, 2011, were used as input to DAPPLE. The PhosphoSitePlus database was downloaded from the URL given earlier, and contained 122031 known phosphorylation sites (104386 of them unique). The International Protein Index (IPI) (Kersey et al., 2004) bovine proteome was downloaded from ftp.ebi.ac.uk/pub/databases/IPI/current in FASTA format and contained 34273 protein sequences. If available, the proteome for each organism represented in the PhosphoSitePlus database was retrieved. The proteomes were downloaded from various sources depending on data availability: the human, mouse, and rat proteomes were downloaded from IPI; the fruit fly proteome was downloaded from UniProtKB; and the dog, ferret, goat, guinea pig, horse, pig, and sheep proteomes were downloaded from GenBank. No proteomes could be downloaded for the remaining organisms represented in the PhosphoSitePlus database (frog, hamster, monkey, quail, rabbit, starfish, and torpedo fish), either because the organism had few or no protein sequences available, or because the organism name refers to a group of organisms (e.g. frog) rather than a single species.

TABLE 2
Comparison of the results of Jalal et al. (2009) with those
of DAPPLE when finding potential phosphorylation sites
in the bovine proteome. The first column indicates the
number of sequence differences between a known phosphorylation
site from the PhosphoSitePlus database, and its best match in the
bovine proteome. The second column
indicates the percentage of known phosphorylation sites with the
indicated number of sequence differences in Jalal et al. (2009).
In this column, the “no homology” row indicates
known phosphorylation sites for which there was either no
match in the bovine proteome, or the annotation
of the match did not match the annotation of the query. The
third column represents output from DAPPLE, with
the “no homology” row indicating that either the
phosphorylation site had no match in the bovine proteome,
or that “RBH?” = “no”. The fourth
column is the same as the third, except instead of using
the RBH method, a known phosphorylation site falls under the
“no homology” column if the hit protein E-value is greater than 10⁻⁵.

| Seq. differences | % (Jalal et al.) | % (RBH) | % (E-value) |
|---|---|---|---|
| 0 | 50% | 28.5% | 33.0% |
| 1 | 13% | 14.7% | 17.3% |
| 2 | 7% | 9.4% | 11.3% |
| 3 | 4% | 6.5% | 7.9% |
| 4 | 1.5% | 4.6% | 5.6% |
| 5 | 0.4% | 3.2% | 4.0% |
| 6 | 0.6% | 1.7% | 2.2% |
| 7+ | 0% | 0.67% | 0.91% |
| No homology* | 22% | 30.9% | 17.8% |

Table 2 compares the summary results given by Jalal and coauthors in Table 1 of their paper with the results produced by DAPPLE. Note that the methodology employed by DAPPLE is not identical to that employed by Jalal et al., so the results that it produces are not expected to be exactly the same. Nevertheless, the percentages of known phosphorylation sites that had a given number of sequence differences with their best bovine BLAST match were similar between the two approaches, with the greatest discrepancies occurring in the percentage of peptides having zero sequence differences. For DAPPLE, the percentage of peptides under the “no homology” category differed depending on the criterion for declaring two proteins as orthologues (see Table 1 caption), with the RBH method being less likely to declare two proteins as orthologues than the E-value method.

The gain in efficiency using DAPPLE compared to manually performing the procedure in Jalal et al. (2009) was considerable. DAPPLE took 63 hours (elapsed time) to run on a Mac OS X machine with a 2.4 GHz Intel Core 2 Duo processor and 4 GB of memory using all 104386 unique phosphorylation sites from the PhosphoSitePlus database. In contrast, manually running the web-based version of BLAST and then recording the results might take five minutes for a single peptide, or over 8,000 hours of labour for all of these known sites. Even the time taken to manually process a small subset of the PhosphoSitePlus data—say, 800 peptides, which was approximately what was used in Jalal et al. (2009)—is around 66 hours, exceeding the time required for DAPPLE to process the entire dataset.

Whereas manually processing 800 peptides would result in a few hundred peptides to choose from for a kinome microarray, the amount of useful information produced by DAPPLE is far greater. For instance, DAPPLE outputs more than 29000 peptides in the cow that have zero mismatches with a known phosphorylation site and for which “RBH?”=“yes”. Downstream selection criteria can therefore be much more restrictive.

The superiority of the orthologue detection procedure employed by DAPPLE can be illustrated using the following example. The human protein with accession number Q9NV56 has the annotation “MRG-binding protein”. A known phosphorylation site from this protein has, as its best match in the bovine proteome, a segment of the protein with accession number E1BHM1, which has the description “C13H20orf20 hypothetical protein LOC616297”. These two proteins are reciprocal BLAST hits and thus orthologues—a fact that would be difficult to ascertain by comparing the annotations. The use of reciprocal BLAST hits also eliminates the subjectivity inherent in comparing annotations. For instance, the two annotations “Guanylyl cyclase-activating protein 2” and “GUCA1B Uncharacterized protein” appear similar, but the two proteins corresponding to the above annotations are not reciprocal BLAST hits. DAPPLE's orthologue detection procedure also has the advantage that the output can easily be filtered so that peptides for which “RBH?”=“no” are eliminated, saving the user a great deal of time comparing annotations.

As kinome microarrays become a more popular tool for studying cellular signaling, the ability to design kinome microarrays suitable for studying different species will become increasingly important. DAPPLE improves upon an already-successful method for designing kinome microarrays. Compared to the previous protocol, it is far less time-consuming and tedious, yet is able to make use of 100 times more information. Through its use of all known phosphorylation sites in the PhosphoSitePlus database, rather than just those from human, DAPPLE is more robust and thorough. Finally, the program greatly improves the ability to identify non-orthologous matches. As such, DAPPLE will be a useful tool for designing species-specific kinome microarrays.

### Detailed Description of DAPPLE Methodology

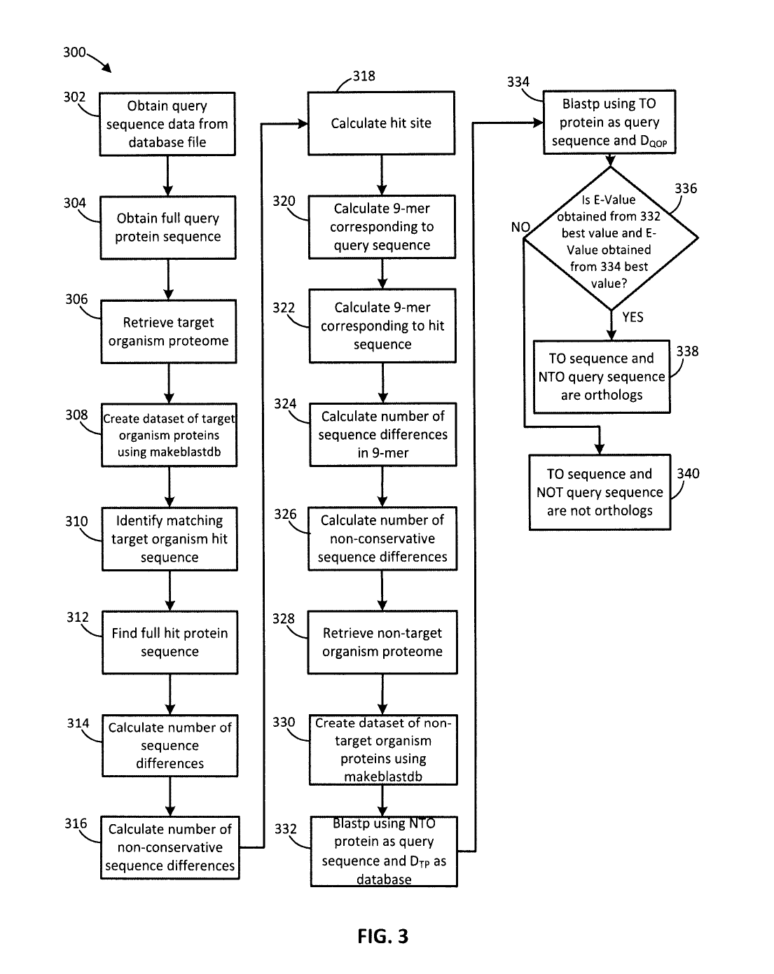

The following and FIGS. 3, 4, 5, 6 and 7 contain a detailed description of the DAPPLE methodology, complemented by a flow chart (FIG. 1) that gives a visual representation of DAPPLE's operation. To make the description easier to understand and more rigorous, symbols are used to refer to the different elements involved in the methodology, such as the target proteome, the known phosphorylation sites, and the protein corresponding to each known phosphorylation site. Many of the symbols correspond to column headings in the output table produced by DAPPLE. Table 3 clarifies the relationship between these symbols and the column headings.

Let K denote the set of known phosphorylation sites. These could be derived from one or more of the following sources: PhosphoSitePlus [Hornbeck et al., 2004], Phospho.ELM [Diella et al., 2004, 2008], PHOSIDA [Gnad et al., 2007], the literature, or any other source of known phosphorylation data. Let QεK be a known phosphorylation site (i.e. sequence of amino acids) from organism Q_{O}, Q_{L} be the length of Q, Q_{A} be the accession number of the full protein corresponding to Q, Q_{F} be the sequence of the full protein with accession number Q_{A}, Q_{C} be the site (residue name and position in Q_{F}; e.g. Y352) of the phosphorylated residue, Q_{LTR} be the number of low-throughput references associated with Q, and Q_{HTR} be the number of high-throughput references associated with Q. Finally, let T be the target organism (the organism for which the user wants to obtain putative phosphorylation sites).

Depending on the source of a given phosphorylation site, some information may not be available. In such cases the information is recorded in the DAPPLE output table as “ND” (“not determined”). For example, currently Q_{LTR} and Q_{HTR} are available only if Q is from the PhospoSitePlus database.

DAPPLE performs the following procedure for each QεK. Referring now to FIG. 3, therein respectively illustrated is a schematic diagram of a method 300 according to the operation of DAPPLE. Some steps of the procedure assume that length of the phosphorylation site Q_{L}=15 and that the middle (eighth) residue is phosphorylated. When Q_{L}<15, which is the case for a small portion of entries in the PhosphoSitePlus database, then some of the information described below (the hit phosphorylation site (H_{C}), the 9-mer sequence differences (U⁹), and the 9-mer non-conservative sequence differences (V⁹)) cannot be determined because it is not known which residue in Q is phosphorylated. In this case, these E-values will be listed as “ND” in the DAPPLE output table.

It will be understood that steps of DAPPLE do not necessarily have to be performed in the order shown in method 300 and according to various embodiments one or more steps may be performed out of order or omitted.

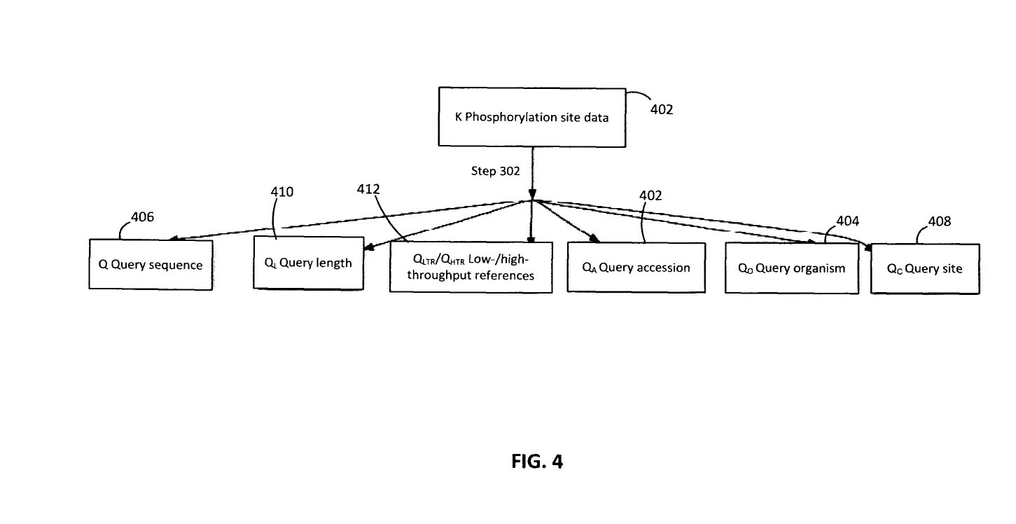

Step 302 Obtain Information from the Phosphorylation Database File.
Referring to FIG. 4, therein illustrated is a diagram showing the data that is extracted from a single K phosphorylation site 401 data. Q_{A} Query accession 402, Q_{O} Query organism 404, Q Query sequence 406, Q_{C} Query site 408, Q_{L} Query length 410, Q_{LTR} Low throughput reference 412, and Q_{HTR} High throughput reference 412 can all be found in a single record in the database file of a K phosphorylation site. As mentioned above, some of this information may only be present if the data come from certain databases. For instance, currently Q_{LTR} 412 and Q_{HTR} 412 are present only if the record is from PhosphoSitePlus.

### Step 304 Obtain the Full Protein Sequence Corresponding to the Query Sequence.

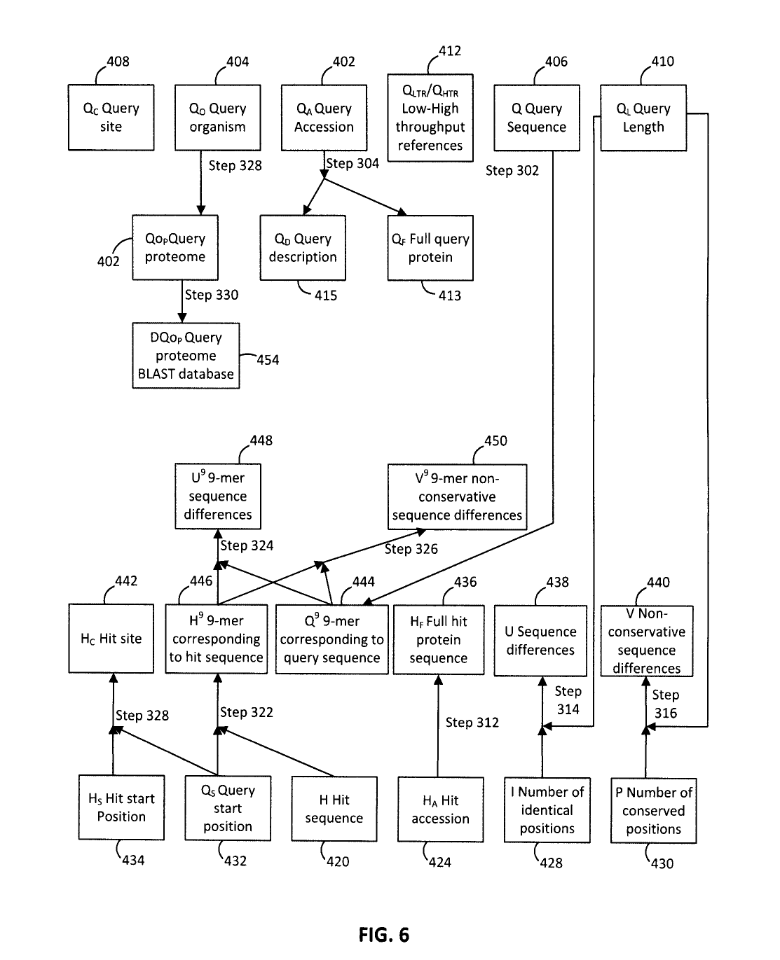

As shown in FIG. 6, use Q_{A} 402 to retrieve Q_{F} Full query protein sequence 413 in FASTA format. This record will also contain the description of this protein QD Query description 415.

### Step 306 Download T_{P} Target Proteome 416, the Proteome of T Target Organism 414.

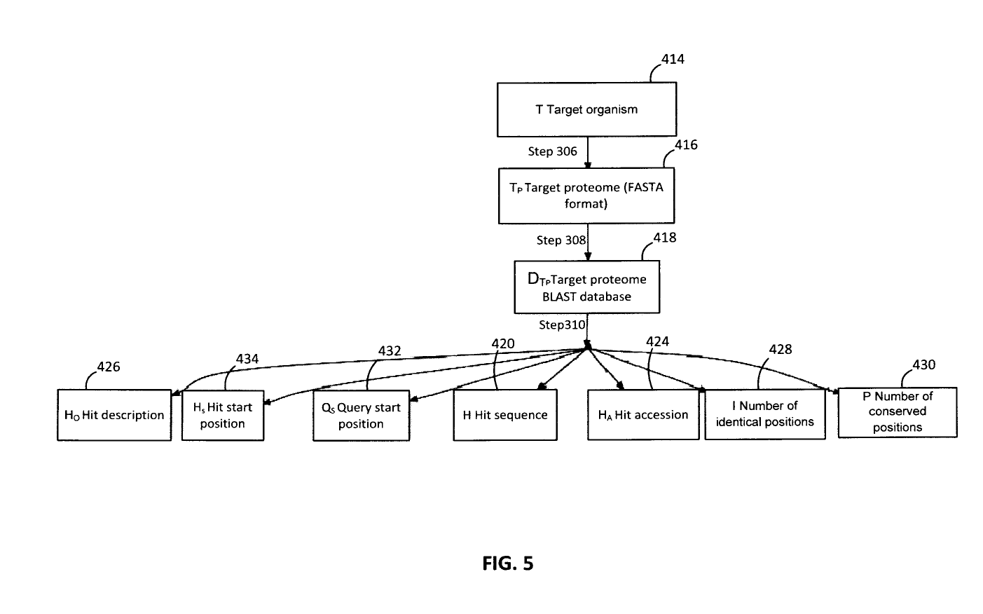

Referring to FIG. 5, therein illustrated is a diagram showing the data that is extracted starting from a T Target organism 414. T_{P} 416 may be downloaded from any online source of protein sequence data, such as GenBank, UniProt, or IPI.

### Step 308 Create a BLAST Database Comprised of the Proteins in T_{P} 416.

Use the makeblastdb program using T_{P} 416 as input to create a BLAST database D_{TP} Target proteome BLAST database 418 (if D_{TP} 418 does not already exist).

TABLE 3
Correspondence between the symbols used above and the column
headings in DAPPLE's output. The column headings are listed
in the order that they appear in the DAPPLE output table.

| Column heading | Corresponding symbol |
|---|---|
| Query-accession | Q_{A}. |
| Query description | Q_{D} |
| Query organism | Q_{O} |
| Query sequence | Q |
| Query site | Q_{C} |
| Hit site | H_{C} |
| Hit accession | H_{A} |
| Hit description | H_{D} |
| Hit sequence | H |
| Sequence differences | U |
| Non-conservative sequence differences | V |
| 9-mer sequence differences | U⁹ |
| 9-mer non-conservative sequence | V⁹ |
| differences | |
| Hit protein rank | S |
| Hit protein E-value | E¹_{F} |
| RBH? | R |
| Low-throughput references | Q_{LTR} |
| High-throughput references | Q_{HTR} |

### Step 310 Find the Most Similar Peptide to Q 406 in T_{P} 416.

Referring to FIG. 5, therein illustrated is a diagram showing the data that is extracted from D_{TP} Target proteome BLAST database 418.
(a.) Run blastp using Q 406 as the query and D_{TP} 418 as the database. The -ungapped option to blastp is used in order to produce an ungapped alignment.
(b) Determine the best match H Hit sequence 420 from the blastp search done in 310. Since BLAST is a local alignment program, H 420 may be shorter than Q 406. The BLAST report also includes H_{A} Hit accession 424 (the accession number of the full protein corresponding to H 420), H_{D} Hit description 426 (the description of that protein), I Number of identical positions 428 (the number of sequence identities in the alignment), P Number of conserved positions 430 (the number of positions in the alignment that are either a match or a conservative substitution), Q_{S} Query start position 432 (the query start position in the BLAST local alignment), and H_{S} Hit start position 434 (the hit start position). Note that Q_{S} 432 is relative to Q 406, whereas H_{S} 434 is relative to H_{F} Full hit protein sequence 436 (the full protein sequence having H_{A} 424 as its accession number). For example, if Q=ABCDEFGHIJKLMNO and the portion of Q that matches with H in the BLAST local alignment is CDEFGHIJKLMN, then Q_{S} (432)=3. If H=CDEYGHIJKLMN and starts at position 263 in H_{F} 436, then H, (434)=263.

### Step 312 Obtain the Full Protein Sequence Corresponding to the Hit Sequence.

Referring back to FIG. 6 use H_{A} 424 to find H_{F} 436 in T_{P} 416.

### Step 314 Find the Number of Sequence Differences Between Q 406 and H 420.

The number of sequence differences U Sequence differences 438 is equal to Q_{L} (410)−I (412).

### Step 316 Find the Number of Non-Conservative Sequence Differences Between Q 406 and H 420.

The number of non-conservative sequence differences V Non-conservative sequence differences 440 is equal to Q_{L} (410)−P (430).

### Step 318 Determine H_{C} Hit Site 442, the Site of the Phosphorylated Residue in H_{F} 436.

The position of this residue can be calculated using the expression H_{S}−Q_{S}+8. As mentioned above, H_{C} 442 cannot be determined if Q_{L}<15.
Step 320 Determine the 9-Amino-Acid-Long Peptide Corresponding to Q 406 with the Phosphorylated Residue as its Central Residue.
The 9-amino-acid-long substring of Q 406 with the phosphorylated residue at its center, denoted Q⁹ 9-mer corresponding to query sequence 444, can be found by taking the substring between indices 4 and 12, inclusive. For example, if Q=ABCDEFGHIJKLMNO, then-Q⁹=DEFGHIJKL.
Step 322 Determine the 9-Amino-Acid-Long Peptide Corresponding to H with the Phosphorylated Residue as its Central Residue.
The 9-amino-acid-long substring of H 424 with the phosphorylated residue at its center, denoted H⁹ 9-mer corresponding to hit sequence 446, can be found by taking the substring between indices (5−Q_{S}) and (13−Q_{S}), inclusive. For example, if H=CZEFGHIJKLMN and Q_{P}=3, then H9=ZEFGHIJKL. If H is less than nine residues long, then H⁹ cannot be computed, along with U⁹ 9-mer sequence differences 448 and V⁹ 9-mer non-conservative sequence differences 450 (see below).

### Step 324 Find the Number of Sequence Differences Between Q⁹ 448 and H⁹ 446.

The number of sequence differences U⁹ 448 is the count of positions where the two residues are different in a gapless alignment between Q⁹ 448 and H⁹ 446. U⁹ 448 cannot be determined if Q_{L}<15 or H is less than nine residues long.

### Step 326 Find the Number of Non-Conservative Sequence Differences Between Q⁹ 444 and H⁹ 446.

The number of non-conservative sequence differences V⁹ 450 is the count of positions where the two residues have a non-positive score in the BLOSUM62 matrix in a gapless alignment between Q⁹ 444 and H⁹ 446. V⁹ 450 cannot be determined if Q_{L}<15 or H is less than nine residues long.

### Step 328 Download Qo_{P} Query Proteome 452, the Proteome of Qo 415.

Qo_{P} 452 may be downloaded from any online source of protein sequence data, such as GenBank, UniProt, or IPI.

### Step 330 Create a BLAST Database DQo_{P} 454 Comprised of the Proteins in Qo_{P} 452.

Use the makeblastdb program using Qo_{P} 452 as input to create a BLAST database DQo_{P} 454 (if DQo_{P} 454 does not already exist and Qo_{P} exists 452). If no proteome exists for Qo_{P} 452, then R 466, which denotes whether or not Q_{F} 413 and H_{F} 436 are reciprocal BLAST hits (see steps 332-340), cannot be computed.

### Steps 332-340 Determine Whether Q_{F} 413 and H_{F} 436 are Reciprocal BLAST Hits.

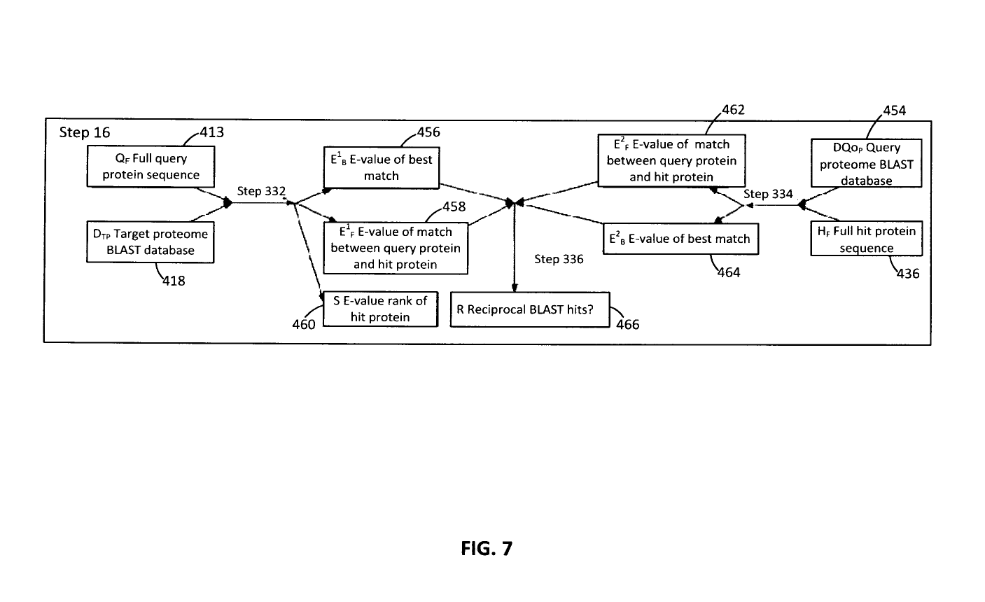

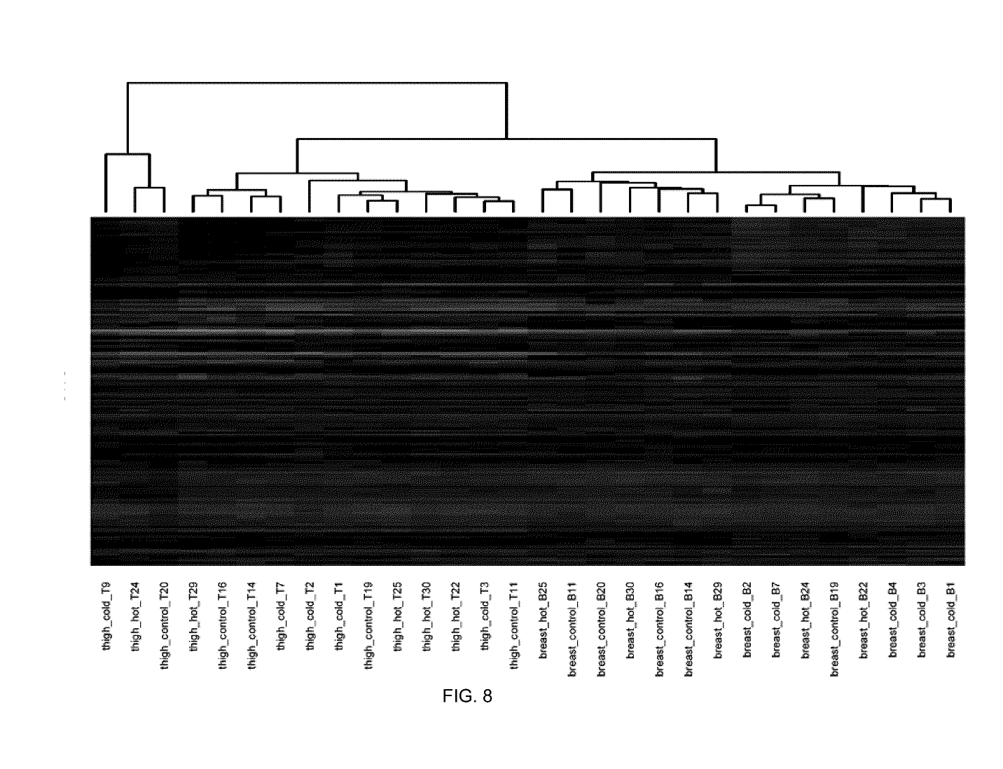

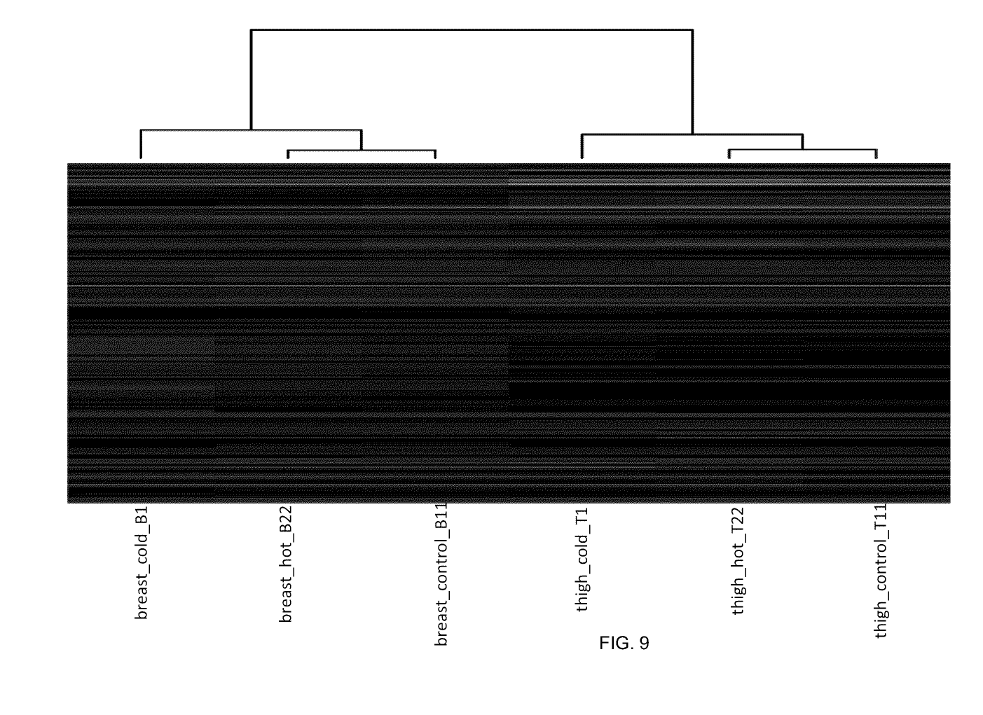

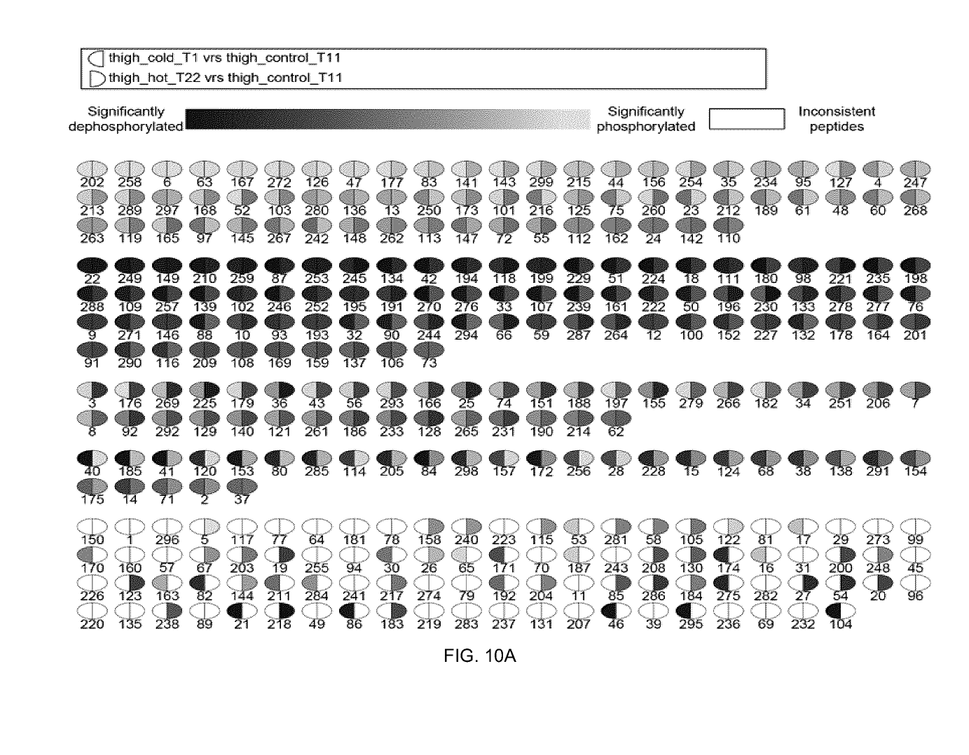

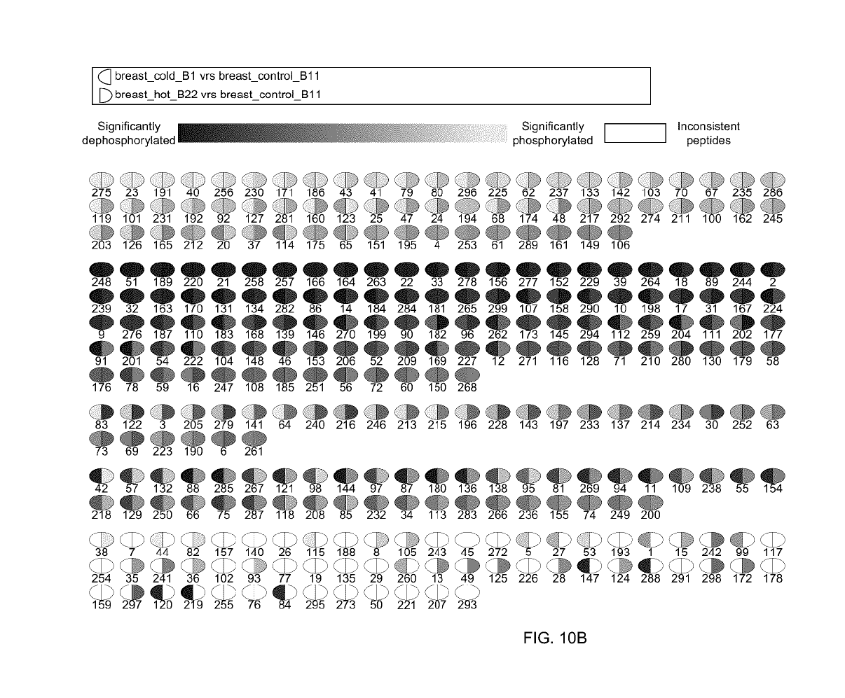

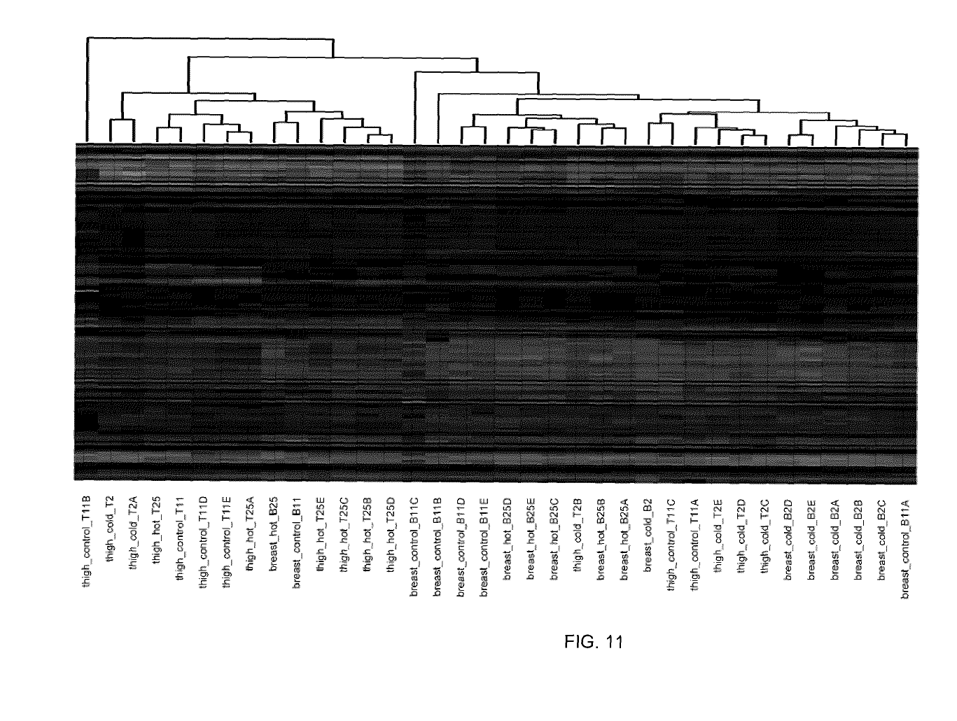

(a) Referring now to FIG. 7, therein illustrated is the flow of data for determining whether Q_{F} 413 and H_{F} 436 are reciprocal BLAST hits. At step 332: Run blastp using Q_{F} 413 as the query and DT_{P} 418 as the database. Determine the E-value E1_{B} 456 of the best BLAST hit, and also the E-value E¹_{F} 458 of the match between Q_{F} 413 and H_{F} 436. Also, let S be the E-value rank of the E¹_{F} 458. In other words, if E¹_{F} is the n^{th} smallest E-value, then S=n.
(b) Step 334 Run blastp using H_{F} 436 as the query and DQo_{P} 454 as the database. Determine the E-value E²_{B} 464 of the best BLAST hit, and also the E-value E²_{F} 462 of the match between Q_{F} 413 and H_{F} 436.
(c) Step 336 Let R=“yes” if Q_{F} 413 and H_{F} 436 are reciprocal BLAST hits (step 338), and “no” otherwise (step 340). If E¹_{B}=E¹_{F} and E²_{B}=E²_{F}, then R=“yes”; otherwise, R=“no”.

Variations of the methods include one or more of the following.

BLAST searches can be parallelized and the computer method (e.g. DAPPLE) can be run on a workstation cluster or computer grid to reduce its computational time.

Second, DAPPLE currently uses only the first match when running BLAST using a known phosphorylation site as the query. However, other matches might be of interest and could be used, especially if the full protein corresponding to one of these matches is orthologous to the full protein corresponding to the query.

Third, DAPPLE currently uses the BLOSUM62 substitution matrix to calculate non-conservative sequence differences. This could be improved by choosing the substitution matrix based on the evolutionary relatedness between the target organism and the organism corresponding to a given known phosphorylation site.

Comparison of ABC and DAPPLE

In ABC, the method for ascertaining orthology (or lack thereof) is based on the E-value between the TO phosphorylation polypeptide sequence when the NTO phosphorylation polypeptide sequence is used as a query against the TO proteome. DAPPLE contains this information as part of its output, so the user can still use the ABC method of ascertaining orthology. DAPPLE additionally comprises a reciprocal BLAST hits method of ascertaining orthology. Table 2 above provides information gathered using a reciprocal blast search and the E-value method. The E-value method can be for example, a more sensitive method of ascertaining orthology, and the RBH method can be more specific.

### Arrays

Peptides corresponding to the TO phosphorylation site sequences can for example be used to make species-specific arrays such as kinome arrays. Accordingly, in another aspect, the disclosure includes a method of making a plurality of species-specific isolated peptides comprising selecting a plurality of matching target organism phosphorylation site sequences according to the method described herein, and synthesizing a plurality of peptides each peptide comprising a sequence of one of the matching target organism phosphorylation site sequences.

The arrays can be for any species, optionally other than for human, rat and mouse. Species-specific arrays designed using methods described herein can be used to address specific biological questions including economically important biological questions. For example, a chicken species-specific array is disclosed comprising a plurality of peptides identified using the methods disclosed herein. Use of such an array is demonstrated.

Temperature stresses which occur during the transport of poultry are important from the perspectives of animal welfare and meat quality. Hot and cold stresses negatively impact the quality of both breast and thigh meat. As the mechanisms of phenotypes cannot be fully explained through traditional biochemical indicators we developed a tool, a chicken-specific kinome peptide array, to provide global insight into cellular signal transduction responses to temperature stress, including post-mortem activities, in chickens. Unique kinomic profiles are observed in breast and thigh tissues, reflecting their distinct cellular phenotypes. Against these backgrounds, in both breast and thigh tissues, greater changes are observed in response to cold, than heat, stress although the specifics of these responses differ in a tissue-specific manner. Metabolic pathways appear upregulated in thigh, and downregulated in breast, in responses to cold stress in living birds. Post mortem time course analysis of these tissues from the temperature stressed birds again verifies the greater impact of cold stress. Collectively this investigation brings forth a valuable tool for characterization of cellular responses in chickens as well as providing specific information to the cellular mechanisms of chickens to temperature stresses.

Transportation of broiler chicken is a stressful, but essential, component of the poultry processing industry. The temperature fluctuations which can occur during transport are of significant consequences to the industry from the perspectives of both animal welfare and meat quality. Both heat and cold stress have been shown to compromise the quality of both breast and thigh meat. Previous research from our group has shown that breast and thigh meat with dark, firm and dry (DFD) characteristics can develop as a result of extreme cold exposure during transportation. DFD incidence in breast and thigh muscles of the cold-stressed birds, accounted as quality defects for the poultry meat industry and resulting in economic loss.

Furthermore, in particular in Canada, the number of dead on arrival (DOA) is often higher in winter, where natural ventilation in trailers has been limited to maintain heat within the load. Paradoxically, this can lead to birds in the middle of the trailers experiencing heat stress while those near cold air ingress points must try to cope with the cold. The high DOA numbers in winter have both welfare and economic implications. The DOA value in Ontario for January 2009 was double the yearly national average representing a loss of over 93,000 birds.

Recent work has shown that the incidence of dark, firm, dry (DFD) breast meat was up to 8% of broilers that experienced cold conditions during transport. The value was even higher in thigh meat which is more sensitive to transportation stresses than breast meat. As both of these meat cuts are of equal value in the marketplace, the resulting inconsistencies in color and eating quality from pale, soft, and exudative (PSE) and DFD can decrease consumer confidence. Heat-stress induced PSE meat is also of lower quality for further processing as the impaired protein functionality leads to poor water holding capacity, cook yield and textural properties.

Traditional metabolic investigations have failed to offer a clear explanation of the mechanisms behind the dramatic drop in core body temperature, survival of birds and incidence of DFD breast and thigh meat in broilers. Specifically investigations failed to identify clear mechanisms or markers which explain these responses to temperature stress in birds. This indicates that novel, likely global, approaches are required to understand these complex, multi-faceted host responses.

There is considerable debate of the most appropriate level to perform characterizations of cellular responses. Transcriptional analysis, based on the experimental maturity of the approach and the relative ease of which arrays can be produced for novel species, is widely used but there are concerns that description of cellular responses at the level of transcription fail to accurately predict or describe cellular responses due to a multitude of post-transcriptional regulatory events. In contrast, protein post-translational modifications, in particular phosphorylation events, occur closer to the phenotype and are often more reliable indicators of phenotypes.

Peptide arrays have proven a valuable tool to enable high throughput characterizations of cellular kinase activity but have been limited to species with well-defined phosphoproteomes. The vast majority of characterized phosphorylation events are for human and mouse which represents a significant obstacle in the application of this approach to non-traditional research animals, including livestock.

The development of a chicken specific peptide array is described. This array consists of 292 peptides representing critical phosphorylation events associated with a broad spectrum of signaling pathways but with particular emphasis on pathways and processes associated with metabolic regulation. Application of these arrays revealed distinctive kinomic profiles associated with breast and thigh tissues and offered specific insight into the cellular changes which occur in these tissues upon exposure of birds to hot and cold stress including a time course investigation of changes which occur post mortem. In both breast and thigh tissues, greater changes are observed in response to cold, than heat, stress although the specifics of these responses differ in a tissue-specific manner. Metabolic pathways appear upregulated in thigh, and downregulated in breast, in responses to cold stress in living birds. Post mortem time course analysis of these tissues from the temperature stressed birds again verifies the greater impact of cold stress.

Peptide Arrays: Design, construction and application of the peptide arrays is based upon a previously reported protocol with modifications (Jalal, 2009). Notably the kinome experiments for all the animals were performed simultaneously in a single run minimizing the possibility of technical variances in the analysis. Briefly, approximately 10×106 cells were collected, pelleted and lysed by addition of 100 μL lysis buffer (20 mM Tris-HCL pH 7.5, 150 mM NaCl, 1 mM EDTA, 1 mM EGTA, 1% Triton, 2.5 mM sodium pyrophosphate, 1 mM Na3VO4, 1 mM NaF, 1 μg/mL leupeptin, 1 g/mL aprotinin, 1 mM PMSF) (all products from Sigma Aldrich unless indicated). Cells were incubated on ice for 10 minutes and spun in a microcentrifuge for 10 minutes at 4° C. A 70 μl aliquot of this supernatant was mixed with 10 μl of activation mix (50% Glycerol, 500 uM ATP (New England Biolabs, Pickering, ON), 60 mM MgCl2, 0.05% v/v Brij-35, 0.25 mg/mL BSA), incubated on the array for 2 hours at 37° C. Arrays were then washed with PBS-(1%) Triton.

Slides were submerged in phospho-specific fluorescent ProQ Diamond Phosphoprotein Stain (Invitrogen) with agitation for 1 hour. Arrays were then washed three times in destain containing 20% acetonitrile (EMD Biosciences, VWR distributor, Mississauga, ON) and 50 mM sodium acetate (Sigma) at pH 4.0 for 10 minutes. A final wash was done with distilled deionized H2O. Arrays were air dried for 20 min then centrifuged at 300×g for 2 minutes to remove any remaining moisture from the array. Arrays were read using a GenePix Professional 4200A microarray scanner (MDS Analytical Technologies, Toronto, ON) at 532-560 nm with a 580 nm filter to detect dye fluorescence. Images were collected using the GenePix 6.0 software (MDS) and the spot intensity signal collected as the mean of pixel intensity using local feature background intensity background calculation with the default scanner saturation level.

### Data Analysis:

Datasets:

The dataset contains the signal intensities associated with each of 292 peptides for the animals under different treatments. For each animal and each treatment, there are nine intra-array replicates. All data processing and analysis was done as per Li, et al. 2012, with the following study specifics.

Animal-Animal Variability Analysis:

For each of the 300 peptides, an F-test was used to determine whether there are significant differences among the three animals under the same treatment condition.

Treatment-Treatment Variability Analysis:

Peptides identified by the F-test as having consistent patterns of response to the various treatments across the three animals were subjected to a paired t-test to compare their signal intensities under a treatment condition with those under control conditions. For each animal-independent peptide, the responses from all three animals were pooled to increase the statistical confidence. Peptides with significant (p<0.10) changes in phosphorylation were identified. This level of significance was chosen to retain as much data as possible and thus facilitate subsequent pathway analysis.

Cluster Analysis:

The preprocessed data were subjected to hierarchical clustering and Principal Component Analysis (PCA) to cluster peptide response profiles across animal-treatment combinations. For each of the 292 peptides in a single treatment and animal, the average was taken over the nine VSN-transformed replicates. For hierarchical clustering, each animal/treatment vector was considered as a singleton (i.e. a cluster with a single element) at the initial stage of the clustering. The two most similar clusters were merged and the distances between the newly merged clusters and the remaining clusters were updated, iteratively. The “Average Linkage+(1−Pearson Correlation)” (Pearson 1996) is the method used, as described by Eisen et al. 1998. It takes the average over the merged (i.e. the most correlated) kinome profiles and updates the distances between the merged clusters and other clusters by recalculating the correlations between them. The first two principal components, namely PC1 and PC2, which account for the largest variability within the sample data, were used to cluster the animal/treatment data points.

Pathway Analysis of Differentially Phosphorylated Peptides:

InnateDB is a publically available resource which, based on levels of either differential expression or phosphorylation, predicts biological pathways based on experiment fold change datasets

(Lynn et al 2008). Pathways are assigned a probability value (p) based on the number of proteins present for a particular pathway as well as the degree to which they are differentially expressed or modified relative to a control condition. For our investigation input data was limited to those peptides selected in the Treatment-Treatment Variability Analysis (above). Since InnateDB requires fold-change (FC) values as input (with p-values optional), the differences between the VSN transformed intensities under control and treatment are converted to fold-change values by the formula 2^{d} where d=averageₜᵣₑₐₜₘₑₙₜ−average_{control}.

Development of a Chicken-Specific Peptide Array:

The chicken-specific peptide arrays were developed through a bioinformatics approach developed by our group termed “Design Array for Phosphorylation-Mediated Experiments (DAPPLE)”. DAPPLE uses genomic information from the species of interest, in this case chicken, as well as publically available information of defined phosphoproteomes to predict phosphorylation sites within the species of interest. There is a moderate degree of conservation of phosphorylation sites between chickens and humans; approximately one quarter of the phosphorylation sites from human were perfectly conserved over a peptide of 15 amino acids (seven residues flanking each side of the phosphoacceptor site) [Table 4]. For the chicken array 292 peptides were selected on the basis of conservation of the phosphorylation sites as well as the interest in the associated biological events [Table 4]. For the final array each peptide is printed in triplicate within each block and each block is printed in triplicate to provide nine technical replicates of each peptide.

Cellular Responses to Temperature Stress:

Groups of chickens (n=5) were exposed to either hot or cold stress. A control group of birds was maintained at room temperature. After the indicated time period birds were sacrificed and tissue samples were collected from the breast and thigh for kinome analysis. Kinome data was processed through PIIKA, an in-house kinome peptide array data processing pipeline described in PCT/CA2011/000764 titled Methods for Kinome Analysis filed Jun. 30, 2011, which is hereby incorporated by reference in its entirety incorporated herein by reference

Cluster analysis of the kinome data demonstrates an absolute tendency for the samples to segregate on the basis of tissue type. This suggests that the cellular phosphorylation-mediated signal transduction occurring within thigh and breast tissue is sufficiently distinct that the samples can be discriminated on the basis of the tissue of origin with a high degree of confidence [FIG. 1]. Given the distinct phenotypes of these tissues this distinction of signaling profiles is not surprising and gives confidence to the ability of the arrays to discriminate distinct signaling patterns within poultry. This also anticipates distinct cellular baselines from which each tissue type will respond to the stressor event.

A closer examination of the clustering results within the breast and thigh samples relieves a strong tendency for the heat-stressed and control samples to cluster together while the samples corresponding to the cold stressed birds cluster distinctly. This occurred within both the breast and thigh samples. This suggests that the breast and thigh samples show a greater cellular response to cold as opposed to heat stress [FIG. 2]. This is consistent with the t-test results: for chicken breast, there are 114 peptides that are both consistent and differentially phosphorylated between cold and control, compared to only 39 for hot versus control. The numbers for thigh are similar: 83 peptides for cold versus control compared to just 36 for hot versus control [FIGS. 3A and 3B].

Pathway Analysis:

To identify conserved cellular responses initiated in chicken breast and thigh muscle following exposure of the animal to hot and cold stress the responses across the five birds were averaged to generate a representative bird for each of the control, cold stressed and heat stressed conditions. Pathway overrepresentation analysis was then performed utilizing InnateDB ( ). Pathways were evaluated based upon the p values for confidence that the pathway is differentially influenced (activated or repressed) under the treatment condition relative to the control as well as the number of differentially phosphorylated peptides within the pathway that supported the involvement of the pathway.

Within the breast and thigh tissues of birds exposed to the heat stress there was a greater number of pathways which were found to be activated rather than repressed [Table 5A]. Within this general common trend there was a unique compliment of pathways which were activated within each tissue. Within breast tissue, heat stress resulted in the activation of a number of calcium regulated events including phosphorylation of CREB through CAMKII as well as calmodulin dependent kinase activation. PGC-1 alpha is a transcriptional coactivator that regulates the genes involved in energy metabolism. This protein interacts with the nuclear receptor PPAR-γ, which permits the interaction of this protein with multiple transcription factors. This protein can interact with, and regulate the activities of, cAMP response element-binding protein (CREB) and nuclear respiratory factors (NRFs). It provides a direct link between external physiological stimuli and the regulation of mitochondrial biogenesis.

Within thigh tissues heat stress activates a distinct compliment of pathways which are also involved in metabolic events including adipocytokine signaling, insulin signaling and MTOR activation [Table 5A]. The mammalian target of rapamycin (mTOR), an evolutionarily conserved serine-threonine kinase, promotes anabolic cellular processes such as protein synthesis in response to growth factors, nutrients (amino acids and glucose), and stress (Biondi et al., 2004; Wullschleger et al., 2006).

In response to cold response the responses between breast and thigh are more divergent. In breast tissue cold stress results in downregulation of a number of pathways associated with metabolic activity including insulin receptor signaling as well as leptin induced signaling [Table 5B]. In contrast the cold stress induced responses in thigh tissues associated with greater metabolic activity (carbohydrate digestion and absorption) as well as activation of cell cycle regulation as well as stress responses [Table 5B].

### Post Mortem Signaling Events Following Temperature Stress:

AMPK:

The importance of understanding thermal stress at the level of phosphorylation-mediated signal transduction activity is supported by the observations that a number of kinases have been specifically implicated in responses to thermal stress. For example, AMP-activated protein kinase (AMPK), which is subject to regulation through phosphorylation, serves to increase the rate of glycolysis. Several studies with a mouse model have also shown that a decrease in AMPK activity resulted in a slower rate of glycolysis due to a slower release of glucose residues from the glycogen stores, resulting in a higher ultimate pH (Shen and Du, 2005). Thus, AMPK has a very important role in both living tissue and in postmortem events. To-date only one study (Sibut et al., 2008) has looked at AMPK activity in poultry, but their results were opposite that observed from work with rats or pigs.

Livestock researchers are faced with highly complex biological problems and are often disadvantaged by an absence of cutting edge research technologies. As a disproportionate amount of research is devoted towards humans and mice, the traditional species of laboratory investigations, so too are the available research tools. Unfortunately, the species-specificity of many of these tools limits that application to investigations of other species. For example, there is an ongoing trend within the field of human medicine to monitor and influence cellular responses at the level of phosphorylation-mediated signal transduction. These phosphorylation reactions are mediated by a class of enzymes called kinases. Kinome analysis, as it has been dubbed, is proving a highly effective strategy for understanding complex biological responses.

Unfortunately, the species-specificity of the kinome research tools has made it exceedingly difficult to apply this perspective to investigations of livestock. To address this limitation our group developed a protocol which enables creation of peptide arrays for kinome analysis of non-traditional animal species (Jalal et al 2009). The genome sequence of the species of interest is the only required prerequisite information. Since then, peptides arrays for cattle, described herein, and honeybees have been developed (for example see PCT [BEE ARRAY] herein incorporated by reference in its entirety). [These arrays will prove to be highly valuable and cost-effective tools in investigations of production-limiting diseases and/or phenotypes of priority to these industries.

The immediate application of these arrays to understand cellular changes associated with events involved in the transport and post slaughter events, for example in describing patterns of signal transduction resulting from hot and cold stresses as well as describing cellular changes which occur post mortem.

Preslaughter transport and handling could increase stress on the birds by decreasing muscle glycogen reserves and therefore affecting the rate and extent of pH decrease, which could affect the resultant meat quality (Owens and Sams 2000; Debut et al., 2003). It is reported that preslaughter temperature affects the postmortem metabolism of muscle via adrenal or other physiological responses or simply by fatigue of the birds (Petracci et al., 2001). Preslaughter heat stress has been reported to accelerate the rate and extent of rigor mortis development (Sams 1999), postmortem glycolysis, and postmortem metabolism and biochemical changes in the muscle, resulting in undesirable changes in meat characteristics similar to the pale, soft, and exudative (PSE) condition (McKee and Sams 1997; Sams 1999; Sandercock et al., 2001). Exposure of chickens to heat stress before slaughter results in breast meat with lower ultimate pH (pHu; Holm and Fletcher 1997; Sandercock et al., 1999), reduced water-binding capacity (WBC; Sandercock et al., 1999; Petracci et al., 2001), and reduced tenderness (Froning et al., 1978; Holm and Fletcher 1997; Petracci et al., 2001). On the other hand, a cold environment before slaughter also causes stress to the bird and may affect meat quality, resulting in meat with dark, firm dry (DFD) characteristics (Dadgar et al., 2010).

TABLE 4
Using sequence homology to identify chicken phosphorylation sites.

| Sequence | All | Peptides |
|---|---|---|
| Differences* | Peptides | on the Array |
| 0 | 10.32 | 95% |
| 1 | 7.42 | 0% |
| 2 | 5.94 | 0% |
| 3 | 4.82 | 0% |
| 4 | 4.14 | 0% |
| 5 | 3.36 | 0% |
| 6 | 2.51 | 0% |
| 7 | 1.30 | 0% |
| 8+ or no match | 59.78 | 5% |

The first column indicates the number of sequence differences between a known phosphorylation site from the PhosphoSitePlus database, and its best match in the chicken proteome. The second column represents, for all sites in these database, the percentage that had that number of sequence differences. The third column represents the percentage of peptides actually chosen for inclusion on the array having a given number of sequence differences

TABLE 5A
Pathway Analysis Hot vs Cold Averaged Animals

| | | Pathway | | | p |
|---|---|---|---|---|---|
| Breast | Up | Calcium signaling pathway | 4 | 3 | 0.072 |
| | | TGF beta Receptor | 4 | 3 | 0.072 |
| | | Bioactive peptide induced signaling pathway | 2 | 2 | 0.086 |
| | | CREB phosphorylation through the activation of | 2 | 2 | 0.086 |
| | | CaMKII | | | |
| | | Ca-calmodulin-dependent protein kinase activation | 2 | 2 | 0.086 |
| | | EPHB forward signaling | 2 | 2 | 0.086 |
| | | Regulation of pgc-1a | 2 | 2 | 0.086 |
| | Down | None | | | |
| Thigh | Up | Adipocytokine signaling pathway | 4 | 4 | 0.031 |
| | | MTOR signaling pathway | 4 | 4 | 0.031 |
| | | TGF beta Receptor | 4 | 3 | 0.082 |
| | | AKT(PKB)-mTOR signaling Insulin receptor | 3 | 3 | 0.082 |
| | | signaling (Mammal) | | | |
| | | Focal adhesion | 3 | 3 | 0.082 |
| | | G beta: gamma signalling through PI3Kgamma | 3 | 3 | 0.082 |
| | | Insulin Pathway | 3 | 3 | 0.082 |
| | | PIP3 activates AKT signaling | 3 | 3 | 0.082 |
| | | Toll-like receptor signaling pathway | 3 | 4 | 0.031 |
| | Down | None | | | |

TABLE 5B

| | | Pathway | | | p |
|---|---|---|---|---|---|
| Breast | Up | C-MYB transcription factor network | 3 | 3 | 0.053 |
| | Down | EGFR1 | 27 | 21 | 0.021 |
| | | Vascular smooth muscle contraction | 7 | 7 | 0.025 |
| | | Insulin receptor signaling (Insulin | 9 | 8 | 0.063 |
| | | receptor signaling) | | | |
| | | Leptin | 11 | 9 | 0.108 |
| Thigh | Up | AKT phosphorylates targets in the cytosol | 3 | 3 | 0.060 |
| | | Aurora A signaling | 3 | 3 | 0.060 |
| | | Carbohydrate digestion and absorption | 3 | 3 | 0.060 |
| | | Cell cycle | 3 | 3 | 0.060 |
| | | FOXM1 transcription factor network | 3 | 3 | 0.061 |
| | | JNK cascade (IL-1 signaling pathway | 3 | 3 | 0.061 |
| | | (through JNK cascade)) | | | |
| | | P53 signaling pathway | 3 | 3 | 0.061 |
| | Down | None | | | |

TABLE 6
List of sequences

| | SEQ | | SEQ | | SEQ |
|---|---|---|---|---|---|
| | ID | | ID | | ID |
| PEPTIDE | NO: | PEPTIDE | NO: | PEPTIDE | NO: |
| AADESVGTMGNRLQR | 1 | ISIRGTLSPKDALTD | 99 | RPRGQRDSSYYWEI | 197 |
| ADTLKERYQKIGDTK | 2 | KEFGVERSLRPMDSS | 100 | RREDKYMYFEFPQP | 198 |
| AEIGEGAYGKVFKAR | 3 | KEPTRRFSTIVVEEG | 101 | RREERSLSAPGNLL | 199 |
| AEKGVPLYRHIADLA | 4 | KEREKEISDDEAEEE | 102 | RREERSMSAPGNLLI | 200 |
| AEPGSNVYLRRELIC | 5 | KESQKSIYYITGESK | 103 | RRLLFYKYVYKKYRA | 201 |
| AGKASFAYAWVLDET | 6 | KILEEVRYIANRFR | 104 | RRSDNEEYVEVGRL | 202 |
| AGVMITASHNRKEDN | 7 | KISEKKMSTPVEVLC | 105 | RSQELRKTFKEIICC | 203 |
| AKEIDVSFVKIEEVI | 8 | KKTVMIKTIETRDGE | 106 | RSRTRTDSYSASQS | 204 |
| AKNAVEEYVYDFRDK | 9 | KLKKEDIYAVEIVGG | 107 | RTHFPQFSYSASIRE | 205 |
| ALRNRSNTPILVDGK | 10 | KLSLNPIYRQVPRLV | 108 | RVEAMKQYQEEIQEL | 206 |
| ALSDHHVYLEGTLLK | 11 | KPGNLLLTTNGTLKI | 109 | RVKGRTWTLCGTPE | 207 |
| APQIQDLYGKVDFTE | 12 | KPIWQRPSKEVEEDE | 110 | RVYAEVNSLRSREY | 208 |
| AQNKLSLTQDPWKV | 13 | KQRRSIISPNFSFMG | 111 | RYMEDSTYYKASKG | 209 |
| AQQCNGIYIWKIENF | 14 | KQWESAYEVIRLKG | 112 | RYPGGESYQDLVQR | 210 |
| ARQSRRSTQGVTLTD | 15 | KRFSFKKSFKLSGFS | 113 | SAGDKVYTVEKADNF | 211 |
| ATKIALYETPTGWK | 16 | KSDISSSSQGVIEKE | 114 | SAVNSRETMFHKER | 212 |
| ATPQRSGSVSNYRSC | 17 | KSFLDSGYRILGAVA | 115 | SCMHRQETVDCLKK | 213 |
| ATYIAGLSGSIVVYMS | 18 | KSIQATLTPSAMKSS | 116 | SDDFDSDYENPDGH | 214 |
| AVKLRGRSFQNNWNV | 19 | KTLGRRDSSDDWEIP | 117 | SDGATMKTFCGTPE | 215 |
| CADVPLLTPSSKEMM | 20 | KVPQRTTSISPALAR | 118 | SDGEFLRTSCGSPN | 216 |
| CASDGKSYDNACQIK | 21 | LAREWHKTTKMSAAG | 119 | SGASTGIYEALELRD | 217 |
| CNENFKKTFKKILHI | 22 | LDAPRLETKSLSSSV | 120 | SGISSVPTPSPLGPL | 218 |
| CTMSVDRYVAVCHPV | 23 | LDGVTTRTFCGTPDY | 121 | SGRDLSSSPPGPYG | 219 |
| DDTSDPTYTSSLGGK | 24 | LERKRPVSMAVMEGD | 122 | SGRKPMLYSFQTSLP | 220 |
| DGATMKTFCGTPEY | 25 | LFRLEQGFELQFRLG | 123 | SGRPRTTSFAESCKP | 221 |
| DGSFIGQYSGKKEKE | 26 | LGGLRISSDSSSDIE | 124 | SIWKGVKTSGKVVW | 222 |
| DGWGKSSDGEDEQQ | 27 | LKKQAAEYREIDKRM | 125 | SKIPLTRSHNNFVAI | 223 |
| DKYFDEQYEYRHVML | 28 | LMKKELDYFAKALES | 126 | SKRHQKFTHFLPRPV | 224 |
| DMSELSSSPPGPYHQ | 29 | LMKTLCGTPTYLAPE | 127 | SKVKRQSSTPNASEL | 225 |
| DPFIDLNYMVYMFKY | 30 | LMTKLRASTTSETIQ | 128 | SLPLTPESPNDPKGS | 226 |
| DRASHASSSDWTPRP | 31 | LPLLVQRTIARTIVL | 129 | SMMHRQETVECLKK | 227 |
| DRGYISPYFINTAKG | 32 | LRRIGRFSEPHARFY | 130 | SMMHRQETVECLRK | 228 |
| DSAKGFDYKTCNVLV | 33 | LSDSYSNTLPVRKNV | 131 | SPIEKVLSPLRSPPL | 229 |
| DSLPCSPSSATPHSQ | 34 | LVDSIAKTRDAGCRP | 132 | SQGGEPTYNVAVGR | 230 |
| DSREDEISPPPPMNPV | 35 | LVTSEASYCKSLNLL | 133 | SQITSQVTGQIGWRR | 231 |
| DSVFCPHYEKVSGDY | 36 | MAHKQIYYSDKYDDE | 134 | SQKKEGVYDVPKSQ | 232 |
| DYNDGRRTFPRIRRH | 37 | MAMKTKTYQVAQMKS | 135 | SQPYSARSRLSAMEI | 231 |
| EADDWLRYGNPWEKA | 38 | MKPGEYSYFSPRTLS | 136 | SQQGMTVYGLPRQV | 234 |
| EDDEKFVSVYGTEEY | 39 | MLRTDLSYLCSRWRM | 137 | SQRQRSTSTPNVHM | 235 |
| EGVRNIKSMWEKGNV | 40 | MPPLIADSPKARCPL | 138 | SQSGMTAYGTRRHL | 236 |
| EKIGEGTYGVVYKAR | 41 | MPPSPLDDRVV | 139 | SREYDRLYEDYTRTS | 237 |
| EKMISGMYMGELVRL | 42 | MRIGAEVYHNLKNVI | 140 | SRLFMHPYELMAKV | 238 |
| ELDELMASLSDFKFM | 43 | MSGTGIRSVTGTPYW | 141 | SRQARANSFVGTAQ | 239 |
| ELWRDPYALKPIRK | 44 | MTPGMKIYIDPFTYE | 142 | SSGSPANSFHFKEA | 240 |
| EPDHYRYSDTTDSDP | 45 | MVKETTYYDVLGV | 143 | SSKIRKLSTCKQQ | 241 |
| EPKSPGEYVNIEFGS | 46 | MVQEAEKYKAEDEKQ | 144 | STFDAHIYEGRVIQI | 242 |
| EPRSRHLSVSSQNTG | 47 | NDTGSKYYKEIPLSE | 145 | STPRRSDSAISVRSL | 243 |
| ERTLYRQSLPPLAKL | 48 | NEYLRSISLPVPVLV | 146 | SVSDQFSVEFEVES | 244 |
| ESTESSNTTIEDEDV | 49 | NKPEDCPYLWAHMKK | 147 | SVSETDDYAEIIDEE | 245 |
| EVLGRGVSSWRRCI | 50 | NLQNGPFYARVIQKR | 148 | TAKTPKDSPGIPPSA | 245 |
| EWSCTRCTFLNPVGQ | 51 | NPLMRRNSVTPLASP | 149 | TDDEMTGYVATRWY | 247 |
| FCDSPPQSPTFPEAG | 52 | NQKKRSESFRFQQEN | 150 | TDGKKVYYPADPVPY | 247 |
| FDKDGNGYISAAELR | 53 | NQVFLGFTYVAPSVL | 151 | TGKENKITITNDKGR | 249 |
| FERADSEYTDKLQHY | 54 | NRFTRRASVCAEAYN | 152 | TGMFPRNYVTPVNR | 250 |
| FGLARAFSLAKNSQP | 55 | NSEESRPYTNKVITL | 153 | THLAWINTPRKQGGL | 251 |
| FIRFDKRSEAEEAIT | 56 | NSQPNRYTNRVVTLW | 154 | THSRIEQYATRLAQM | 252 |
| FTSIGEDYDERVLPS | 57 | PASSAKTSPAKQQAP | 155 | TKIPLIKSHNDEVAI | 253 |
| GDLVIVLTGWRPGSG | 58 | PEFRIEDSEPHIPLI | 156 | TKSIYTRSVIDPIPA | 254 |
| GEENAVLYQNYKEKA | 59 | PEGEKLHSDSGISVD | 157 | TPPRRAPSPDGFSPY | 255 |
| GETAKGDYPLEAVRM | 60 | PEPESEESDLEIDNE | 158 | TRGQPVLTPPDQLVI | 256 |
| GEVQRRLSPPECLNA | 61 | PEQSKRSTMVGTPYW | 159 | TVGNKLDTFCGSPPY | 257 |
| GGPEPGPYAQPSINT | 62 | PETEENIYQVPTSQK | 160 | TVPESIHSFIGDGLV | 258 |
| GINPCTETFTGTLQY | 63 | PGEDFPASPQRRNTS | 161 | TVQNALQTPCYTPYY | 258 |
| GIPVRCYSAEVVTLW | 64 | PGRMRRSSLTPLAST | 162 | TYIDPHTYEDPNQAV | 260 |
| GIPVRVYTHEVVTLW | 65 | PIEQLLDYNRIRSGM | 163 | VFDLGGGTFDVSLLT | 261 |
| GKGTPLGTPATSPPP | 66 | PKIHRSASEPSLNRA | 164 | VIGIDLGTTNSCVAV | 262 |
| GLKVGVSSRINEWLT | 67 | PLCMITEYMENGDLN | 165 | VIRLKGYTNWAIGLS | 263 |
| GLSSAMCYSALVTKT | 68 | PNRQRIRSCVSAENF | 166 | VKILTGFYQDFEKIS | 264 |
| GLSSSPSTPTQVTKQ | 69 | PPPRSHVSMVDPNES | 167 | VLDIEQFSTVKGVNL | 265 |
| GNRTTPSYVAFTDTE | 70 | PPSREAQYNNFAGNS | 168 | VLGTDELYGYLKKYH | 266 |
| GNWRRGATAGGCRNY | 71 | PREKRRSTGVSFWTQ | 169 | VLNTHRKSLNLVDIP | 267 |
| GPEKDHVYLQLHHLP | 72 | PRPEHTKSIYTRSVI | 170 | VLSSRKLSLQERSSG | 268 |
| GPRVWFVSNIDGTHI | 73 | PTVIHKHYQIHRIRQ | 171 | VLVRHGESAWNLEN | 269 |
| GRKRKMRSKKEDSSD | 74 | PVFDLTATPKGGTPA | 172 | VPVEITISLLKRAMD | 270 |
| GSPGMKIYIDPFTYE | 75 | QAASNFKSPVKTIR | 173 | VSGQLIDSMANSFVG | 271 |
| GSPNRVYTHQVVTRW | 76 | QAQPRQDYLKGLSII | 174 | VSTQLVNSIAKTYVG | 272 |
| GTEPKIKYYSELCAP | 77 | QAQRFRFSPMGVDHM | 175 | VTVSLSLTAKRMAKK | 273 |
| GVPVRTYTHEVVTLW | 78 | QDNDQPDYDSVASDE | 176 | VWDHIEVSDDEDETH | 274 |
| GYNAREYYDRIPELR | 79 | QELHDIHSTRSKERL | 177 | VYIDPFTYEDPNEAV | 275 |
| HDLKRCQYVTEKVLA | 80 | QGTGTNGSEISDSDY | 178 | WEQGQADYMGVDSF | 276 |
| HFDPVTRSPLTQDQL | 81 | QLVKMLLYTEVTRYL | 179 | WGLNKQGYKCRQCN | 277 |
| HKDKFLQTFCGSPLY | 82 | QMVNGAHSSSTLDEA | 180 | WNLENRFCGWYDAD | 278 |
| HQLFRGFSFVATGLM | 83 | QNTRDHASTANTVDR | 181 | WRLNERHYGALTGL | 279 |
| HRLRRRGSTVPQFTN | 84 | QRQRSTSTPNVHMVS | 182 | WSKVVLAYEPVWAIG | 280 |
| IDNIFRFTQAGSEVS | 85 | QSDFEGFSYVNPQFV | 183 | WVRKTPWYQ | 281 |
| IEDDIIYTQDFTVPG | 86 | QVKIWRRSFDIPPPP | 184 | WYDNEFGYSNRVVD | 282 |
| IEHIGLLYQEYRDKS | 87 | RAIGRLSSMAMISGM | 185 | YDWMRRVTQRKKIS | 283 |
| IEKIGEGTYGVVYKG | 88 | RAVRRLRTACERAKR | 186 | YIEDEEYYKASVTRL | 284 |
| IEKRYRSSINDKIIE | 89 | RDVYDKEYYSVHNKT | 187 | YIGNLNESVTPADLE | 285 |
| IERLRTHSIESSGKL | 90 | RFHGRAFSDPFVQAE | 188 | YIQEWQYIKRLEDA | 286 |
| IGVMVTASHNPEEDN | 91 | RGAPVNVSSSDLTGR | 189 | YQRSKSLSPSQLGYQ | 287 |
| IKLECVKTKHPQLHI | 92 | RGEPNVSYICSRYYR | 190 | YSFQMALTSVVVTLW | 288 |
| ILHRYYRSPLVQIYE | 93 | RKMKDTDSEEEIREA | 191 | YSHKGHLSEGLVTK | 289 |
| IPEPAHAYAQPQTTS | 94 | RLDGENIYIRHSNLM | 192 | YSLQISSIPLYKKKE | 290 |
| IQRIMHNYEKLKSRI | 95 | RLLAGPDTDVLSFVL | 193 | YSSSQRVSSYRRTFG | 291 |
| IREAMTAYNSHEEGR | 96 | RNGRKHASILLRKKD | 194 | YVHVNATYVNVKCVA | 292 |
| ISEDIKSYYTVRQLE | 97 | RPHFPQFSYSASGTA | 195 | | |
| ISGYLVDSVAKTMDA | 98 | RPPGRPISGHGMDSR | 196 | | |

Accordingly as demonstrated above with the chicken species array, peptides corresponding to the TO phosphorylation site sequences can for example be used to make species-specific arrays such as kinome arrays. Accordingly, in another aspect, the disclosure includes a method of making a plurality of species-specific isolated peptides comprising selecting a plurality of matching target organism phosphorylation site sequences according to the method described herein, and synthesizing a plurality of peptides each peptide comprising a sequence of one of the matching target organism phosphorylation site sequences

In another aspect, the disclosure includes a method of making a species-specific array comprising selecting a plurality of matching target organism phosphorylation site sequences according to the method described herein, synthesizing a plurality of peptides each peptide comprising a sequence of one of the matching target organism phosphorylation site sequences and attaching the plurality of peptides to a substrate surface.

In an embodiment, the method is for making a plurality of bovine specific peptides and/or a bovine specific array. In another embodiment, the method is for making a plurality of chicken specific peptides and/or a chicken specific array.

The methods were used to identify a number of bovine and chicken specific peptides and design a bovine specific and a chicken specific array. Accordingly the plurality of peptides and/or array can be determined or designed for any species for which proteome sequence exists. A bee species-specific peptide array and uses thereof is described in PCT/IB2012/001254 filed Jun. 24, 2012 titled METHODS AND COMPOSITIONS FOR CHARACTERIZING PHENOTYPES USING KINOME ANALYSIS, which is hereby incorporated by reference.

Species-specific isolated peptides and species-specific arrays are useful for identifying economically important traits. For example, the chicken species-specific array was demonstrated to be useful for probing responses to shipping stress and could be used to identify markers associated with desirable traits (e.g. increased resistance to shipping stress). The arrays can be used to obtain phosphorylation profiles and for classifying chickens with desirable characteristics.

Accordingly, in other aspects, the disclosure includes an isolated peptide whose sequence is identified using a method described herein, a plurality of said peptides (for example a plurality of isolated peptides) that are specific for a species and a species-specific array comprising a plurality of peptides attached to a substrate surface, each peptide comprising a sequence of one of a matching target organism phosphorylation site sequence selected according to a method described herein, wherein the similarity corresponds to or is below a preselected threshold.

In an embodiment, the isolated peptide comprises an isolated chicken peptide (e.g. peptides found in chicken). In another embodiment, the plurality of peptides is a plurality of chicken species peptides. In an embodiment, the array is a chicken specific array.

In an embodiment, each isolated peptide comprises a sequence of about 5 to about 100 amino acids, for example about 5 to about 50 amino acids or about 5 to about 30 amino acids, optionally wherein the sequence comprises a contiguous sequence present in a peptide sequence selected from the group of SEQ ID NOs: 1 to 292, said contiguous sequence comprising a chicken phosphorylation site sequence. For example, each of the sequences in Table 6 (SEQ ID NOs: 1-292) comprise a chicken phosphorylation site sequence. The isolated peptide for example comprises minimally about 6 amino acids and the portion of a sequence in Table 6 that comprises said phosphorylation site sequence.

Each peptide for example comprises at least one serine, threonine or tyrosine amino acid residue.

Each of the peptides comprising sequences selected from Table 6, can for example, comprise 5, 6, 7, 8, 9, 10, 11, 12, 13, 14, 15, 16, 17, 18, 19, 20, 21, 22, 23, 24, 25, 26, 27, 28, 29 or 30 or more amino acids. For example, if SEQ ID NO:1 is selected, the peptide can comprise 8, 9, 10, 11, 12, 13, 14 or 15 of SEQ ID NO:1 as long as the phosphorylation site is included. Preferably, the phosphorylation site is centered or about centered in the peptide length selected. Typical phosphorylatable amino acids include serine, threonine and tyrosine residues.

The peptides can also for example comprise linkers (e.g. flexible linkers) or other sequence not present in the surrounding sequence, for example for attaching to a support surface.

In another aspect, the disclosure includes a plurality of peptides (e.g. a collection), each peptide comprising a sequence of about 5 to about 100 amino acids, for example about 5 to about 50 amino acids or about 5 to about 30 amino acids, optionally wherein the sequence comprises a contiguous sequence present in an amino acid sequence selected from the group of SEQ ID NOs: 1 to 292, said contiguous sequence comprising a chicken phosphorylation site sequence.

In an embodiment, the plurality of peptides comprises at least 25 peptides, at least 50 peptides, at least 100 peptides, at least 200 peptides, at least 300 peptides, at least 400 peptides, at least 500 peptides or at least 1000 peptides or any number in between. In an embodiment, each peptide has a sequence of a matching target organism phosphorylation site sequence.

In an embodiment, the plurality of peptides comprises a subset (e.g. two or more) of the peptides or parts thereof (the parts comprising a chicken phosphorylation site sequence) listed in Table 6, for example, about 5, 10, 15, 20, 25, 50, 75, 100, 125, 150, 175, 200, 225, 250, 275 or 292 of the peptides listed in Table 6. In an embodiment, the plurality of peptides comprises a subset (e.g. 2 or more) of the peptides listed in Table 6. In a further embodiment, the plurality of peptides comprises the set of peptides in Table 6.

Each of the plurality of peptides is for example an isolated peptide, for example an isolated synthetic chemically peptide synthesized using for example commercially available methods and equipment. Methods of synthesizing peptides are well known in the art, and include for example liquid phase peptide synthesis and solid phase peptide synthesis (SPPS), including for example Fmoc SSPC and Boc SPPS.

In another embodiment, the plurality of peptides (e.g also referred to as peptide targets) is attached to a support surface, each peptide comprising a sequence of a chicken phosphorylation site sequence selected for example according to a method described herein, wherein the similarity is below a preselected threshold.

Additional chicken specific sequences (e.g. not listed in Table 6) identified using the described methods can also be included in the plurality of peptides. Further specific subsets of phosphorylation targets can selected for inclusion in the plurality.

A further aspect includes a composition comprising one or more peptides listed in Table 6 and a diluent. The peptide can for example be attached to a bead or spotted on a slide and can for example be used in methods described herein. In an embodiment, the composition comprises 1 to 292 peptides listed in Table 6, or any number of peptides between 1 and 292.

In another aspect, the disclosure includes an array comprising a plurality of peptides. In an embodiment, the array comprises a plurality of peptides, each comprising an amino acid sequence of about 5 to about 100 amino acids, for example about 5 to about 50 amino acids or about 5 to about 30 amino acids, optionally wherein the sequence comprises a contiguous sequence present in an amino acid sequence selected from the group of SEQ ID NOs: 1 to 292, said contiguous sequence comprising a chicken phosphorylation site sequence.

Generally, since the peptide molecules are typically pre-formed and spotted onto the support as intact molecules, they are comprised of 5 or more amino acids, and are peptides, polypeptides or proteins. For the most part, the peptide molecules in the present arrays comprise about 5 to 100 amino acids, for example 5 to 50 amino acids, preferably about 5 to 30 amino acids. A phosphorylation motif comprises for example 4 amino acids. The amino acids forming all or a part of a peptide molecule may be any of the twenty conventional, naturally occurring amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y).

In an embodiment, each of the array plurality of peptides comprises a sequence that is about 8 to about 15 amino acids of a peptide sequence selected from SEQ ID NO: 1-292.

In an embodiment, the peptide array comprises at least 2 peptides, at least 3 peptides, at least 4 peptides, at least 5 peptides, at least 25 peptides, at least 50 peptides, at least 100 peptides, at least 200 peptides, at least 300 peptides, at least 400, at least 500 or at least 1000 or any number in between 2 and 1000. Each peptide is optionally spotted in at least two replicates, or at least 3 replicates per array, optionally as replicate blocks. For example, the peptide can be spotted, 4, 5, 6, 7, 8 or 9 times or more. For example up to 15 replicates.

In another embodiment, the array comprises a plurality of peptides each peptide comprising a peptide sequence selected from the group listed in Table 6.

Each peptide (e.g. target peptide) corresponds to a protein which can be identified for example by an accession number.

Subsets of the plurality of peptide can be selected for inclusion on the array. For example, depending on the dataset to be obtained, the plurality of peptides can comprise peptides with known phosphorylation motifs, optionally phosphorylation motifs for proteins that are found in a signaling pathway or related pathways. For example, as indicated for the chicken specific array, peptides corresponding to proteins involved in metabolic pathways were selected.

The plurality of peptides can also comprise for example peptide sequences of a selected group of molecules, for example proteins involved in immune responses, specific signaling cascades or can be related molecules, e.g. sharing a particular sequence identity.

Such peptide arrays can be useful for deciphering peptides phosphorylated or signaling pathways activated by a stressor such a physical treatment (e.g. cold/hot stress), an infectious agent or a macromolecule. Alternatively, the peptide array can comprise random peptide sequences comprising putative phosphorylation sites wherein the plurality of peptides or a subset thereof comprises at least one of a serine, threonine or tyrosine residue.

In an embodiment, the array further comprises a negative control peptide and/or a positive control peptide. In an embodiment, the negative control peptides do not contain any Ser, Thr or Tyr residues. Positive control peptides could include for example peptides comprising phosphorylation sites of histones 1 through 4, bovine myelin basic protein (MBP), and/or α/β casein. Alternatively, the peptides can be either random sequences (e.g. control peptide), not necessarily always containing a Ser/Thr or Tyr, or represent known or predicted phosphorylation sites (e.g. peptides comprising Ser/Thr or Tyr residues).

In an embodiment the control peptide is selected according to a selected test condition. For example, a negative control could be an irrelevant peptide sequence optionally containing a T, Y, or S amino acid at the centre position. A positive control could be for example a peptide corresponding to a protein known to be phosphorylated by a given treatment in the experiment. The positive controls can be any length for example, they can be full length proteins.

Any of the non-phosphorylation site amino acids in the peptide molecules may be replaced by a non-conventional amino acid. In general, conservative replacements are preferred. Conservative replacements substitute the original amino acid with a non-conventional amino acid that resembles the original in one or more of its characteristic properties (e.g., charge, hydrophobicity, stearic bulk; for example, one may replace Val with Nval). The term “non-conventional amino acid” refers to amino acids other than conventional amino acids, and include, for example, isomers and modifications of the conventional amino acids, e.g., D-amino acids, non-protein amino acids, post-translationally modified amino acids, enzymatically modified amino acids, constructs or structures designed to mimic amino acids (e.g., .alpha,.alpha.-disubstituted amino acids, N-alkyl amino acids, lactic acid, .beta.-alanine, naphthylalanine, 3-pyridylalanine, 4-hydroxyproline, O-phosphoserine, N-acetylserine, N-formylmethionine, 3-methylhistidine, 5-hydroxylysine, and nor-leucine). The peptidic molecules may also contain nonpeptidic backbone linkages, wherein the naturally occurring amide —CONN— linkage is replaced at one or more sites within the peptide backbone with a non-conventional linkage such as N-substituted amide, ester, thioamide, retropeptide (—NHCO—), retrothioamide (—NHCS—), sulfonamido (—SO.sub.2NH—), and/or peptoid (N-substituted glycine) linkages. Accordingly, the peptide molecules of the array include pseudopeptides and peptidomimetics. The peptides can be (a) naturally occurring, (b) produced by chemical synthesis, (c) produced by recombinant DNA technology, (d) produced by biochemical or enzymatic fragmentation of larger molecules, (e) produced by methods resulting from a combination of methods (a) through (d) listed above, or (f) produced by any other means for producing peptides.

A peptide can for example comprise up to 1, 2 3, 4, or up to 5 conservative changes for every 15 amino acid sequence. For example, each peptide can comprise up to 70%, 75%, 80%, 85%, 90%, 95% sequence identity with a peptide selected from Table 6.

The chicken specific array can be used to measure protein kinase activity in a chicken sample, for example for analyzing cellular signaling events, for example under test conditions. The array enables for example investigation of phosphorylation-mediated signal transduction activity in a sample from a chicken and can be used to identify biomarkers for marker assisted selection and/or to understand some of the biology associated with particular phenotypes. For example, the arrays can be used to identify chicken phenotypes that have increased tolerance to a stressor and/or to identify strategies that reduce stress response. For example, it is demonstrated that signaling changes occur upon cold and heat stress in chickens. Chickens exhibit differences in cellular signalling pathways discernable using an array comprising chicken specific peptides comprising known or putative phosphorylation sites. The arrays can be used to identify conditions that minimize the stress for example what time and temperatures can be tolerated with minimal stress induction. The methods can also be used to identify phenotypes that are more resistant to a stressor. For example, the profiles obtained for a specific phenotype are reproducible and specific profiles can be obtained for use in identifying chickens of unknown or otherwise unconfirmed characteristics. Chickens having the desired phenotype can then be cross-bred according to the desired traits.

For example the technology can be applied to chicken breeding programs and used to identify phenotypes of interest for example susceptibility/resistance to pathogenic organisms and/or cellular responses to infection or stressors.

A further aspect comprises a method of determining a phosphorylation profile of a test sample comprising:

a) incubating a species-specific array comprising a plurality of peptides, wherein the plurality of peptides are selected according to a method described herein, with the test sample to provide a test array and optionally incubating a second array with a comparator sample such as a control sample or a second test sample to provide a comparator array; and

b) measuring a phosphorylation level signal intensity for each of the plurality of peptides for the test array and optionally the comparator array, the phosphorylation level signal intensity resulting from the interaction of the sample with each of the plurality of peptides;

to provide the phosphorylation profile.

In an embodiment, the phosphorylation profile comprises a plurality of data values, for example, each value representing a phosphorylation level of a peptide and/or the direction of change (e.g. an indication of increased or decreased phosphorylation level of one or more of the plurality of peptides on the test array compared to the comparator array or internal control) and/or the magnitude of said increase or decrease.

The increase or decrease can for example be relative to an internal control or controls, e.g. relative to background. Alternatively, the increase or decrease can be relative to a comparator array such as a control array contacted with a suitable control sample or a different test sample, e.g. which is treated differently or comprises a different test subject.

In an embodiment, the method for determining a phosphorylation profile for a sample optionally from a subject, said method comprising the steps of:; a) incubating a sample optionally obtained from said subject with ATP and/or other suitable ATP source and a plurality of peptides, for example, wherein each of the plurality comprises a sequence of about 5 to about 100 amino acids, for example about 5 to about 50 amino acids or about 5 to about 30 amino acids, wherein the sequence comprises a contiguous sequence present in a peptide sequence selected from Table 6, wherein said contiguous sequence comprises a chicken phosphorylation site sequence; and, b) measuring for each peptide a phosphorylation level signal intensity resulting from the interaction of the sample with the plurality of peptides, thereby providing a phosphorylation profile for the sample.

In an embodiment, the method further comprises calculating the direction and/or magnitude of change compared to an internal control or a comparator array.

In an embodiment, the sample is from a subject and the method further comprises first obtaining a sample from the subject.

The plurality of peptides incubated with the sample can for example be any plurality of peptides described herein, including for example peptides attached to a solid support such as in an array. Accordingly in an embodiment, the plurality of peptides is comprised in an array described herein.

In another embodiment, the plurality of peptides is comprised in a composition that is contacted with ATP and/or other suitable ATP source and the level of phosphorylation is detected by a method known in the art. For example, the composition can be separated electrophoretically and probed with a phosphospecific antibody, or visualized using labeled ATP of a phosphor specific stain. Slot blots, immunohistochemical and the like can also be used. This method can be used for example with a subset of peptides and/or corresponding proteins are being assessed for example about 2, 3, 4, 5, 6 to 10, 11-15 or more peptides or corresponding proteins.

A compound that functions as ATP can also be used instead of ATP in the methods described. For example, other suitable ATP sources such ATP analogs can be used. GTP can also be used in place of ATP or ATP source.

The sample from the subject can alternatively be a cell sample from a cell line, for example treated with a stressor.

Kinotyping can be used for identifying cell, tissue and organism level phenotypes. Accordingly, in an embodiment, an array comprising a plurality of peptides or parts thereof selected from Table 6 is used to identify a chicken cell, chicken tissue or chicken at the organism level, phenotype.

In an embodiment, the method comprises: a) determining a detectable phosphorylation profile of a sample obtained from the subject, said phosphorylation profile resulting from the interaction of said sample with a plurality of peptides described herein; b) comparing said phosphorylation profile to one or more reference phosphorylation profiles, each reference phosphorylation profile corresponding to a known phenotype and c) classifying the subject according to the probability of said phosphorylation profile falling within a class defined by said reference phosphorylation profile.

In an embodiment, the method for classifying a subject for example as having or not having a phenotype, comprises a) obtaining a sample of the subject; b) incubating said sample with ATP and/or other suitable ATP source and a plurality of peptides, for example comprising sequences or parts thereof selected from Table 6 and/or other peptides, each peptide comprising a phosphorylation site sequence; and c) determining a detectable phosphorylation profile, said phosphorylation profile resulting from the interaction of the sample with the plurality of peptides; d) comparing said phosphorylation profile to one or more reference phosphorylation profiles of a known phenotype (e.g. one or more phenotype reference phosphorylation profiles); wherein a difference or a similarity in the phosphorylation profile of the plurality of peptides between the sample and said one or more reference phosphorylation profiles is used to classify the subject for example as having or not having the phenotype.

In an embodiment, the similarity is assessed by calculating a measure of similarity.

The subject is identified as having or likely having the phenotype of the phenotype reference phosphorylation profile most similar to said subject phosphorylation profile. For example, if a subject has a higher similarity to a first phenotype reference phosphorylation profile, the subject is identified as having said first phenotype; if a subject has a higher similarity to a second phenotype reference phosphorylation profile, the subject is identified as having said second phenotype. The phosphorylation levels can also be used to determine a threshold, wherein if a subject is above or below a threshold, the subject is identified as having the phenotype corresponding to above or below the threshold.

In an embodiment, the method of classifying a subject comprises: (i) calculating a first measure of similarity between a first phosphorylation profile, said first phosphorylation profile comprising the phosphorylation levels of a plurality of peptides described herein, in a cell sample taken from said subject and a first phenotype reference phosphorylation profile, said first phenotype reference phosphorylation profile comprising phosphorylation levels of said plurality of peptides that are for example, average levels of said respective peptides in cells of a plurality of subjects having said first phenotype; and (ii) classifying said subject as having the first phenotype if said first phosphorylation profile has a similarity to said first phenotype reference phosphorylation profile that is above a predetermined threshold, classifying said subject as not having said first phenotype if said first phosphorylation profile has a similarity to said first phenotype reference phosphorylation profile that is below a predetermined threshold,

In an embodiment, step (i) further comprises: calculating a second measure of similarity between said first phosphorylation profile and a second phenotype reference phosphorylation profile, said second phenotype reference phosphorylation profile comprising phosphorylation levels of said plurality of peptides that are average phosphorylation levels of the respective peptides in cells of a plurality of subjects having said second phenotype; and classifying said subject as having said second phenotype if said first phosphorylation profile has a similarity to said first phenotype reference phosphorylation profile that is below a predetermined threshold and said first phosphorylation profile has a similarity to said second phenotype reference phosphorylation profile that is above a predetermined threshold.

Similarity can be determined for example using clustering analysis.

Similarity can also be determined by calculating a similarity score or threshold.

In a further embodiment, the method includes displaying; or outputting to a user interface device, a computer-readable storage medium, or a local or remote computer system, the classification produced by said classifying step.

The phosphorylation profile can be determined using known methods for example methods for array analysis. In particular, the phosphorylation profile can be determined using methods described in PCT/CA2011/000764 titled Methods for Kinome Analysis filed Jun. 30, 2011, which is hereby incorporated by reference in its entirety.

PCT/CA2011/000764 describes for example, the signal intensities measuring specific phosphorylation events of the peptides on a kinome array are subjected to variance stabilization transformation to bring all the data onto the same scale while alleviating variance-mean-dependence. Spot-spot and subject-subject variability are examined using χ² and F-tests to identify and eliminate inconsistently regulated peptides due to technical and biological factors of the experiments, respectively. One-sided paired t-test is used to identify differentially phosphorylated peptides relative to the control from the preprocessed kinome data. The information from the differential peptides can be used to probe gene ontology (GO) annotations and known signaling transduction pathways from online database to discover treatment-specific cellular events from various biological aspects. For comparative visualization of the global kinome profiles induced by selected stimuli, hierarchical clustering and principal component analysis are applied to the data after averaging the replicate intensities. The results from the differential analyses and clustering are compared to draw further insights from the data and/or to classify subjects. The results can be presented for example in pseudo-images generated based on the p-values from the one-sided t-tests for phosphorylation or dephosphorylation of each peptide. Each peptide is represented for example by one small colored circle. The depths of the coloration in the colors, for example red and green, are inversely related to the corresponding p-values.

A further aspect includes a kit comprising a plurality of peptides described herein comprising sequences present in a peptide selected from Table 6, an array comprising a support and the plurality of peptides, and/or a kit control.

In an embodiment, the kit further comprises instructions for use.

In an embodiment, the kit comprises about 5, 10, 15, 20, 25, 30, 35, 40, 50, 60, 70, 80, 90, 100, 125, 150, 175, 200, 225, 250, 275, 300 or more peptides, optionally selected from Table 6. In an embodiment, the peptides are comprised in a composition, or attached to a solid support such as in a microarray.

Another aspect includes a phosphorylation profile comprising for each of a plurality of peptides selected from Table 6, one or more phosphorylation characteristics, for example signal intensities, fold change, and/or phosphorylation status, associated with a phenotype and/or treatment.

While the present disclosure has been described with reference to what are presently considered to be the preferred embodiments, it is to be understood that the disclosure is not limited to the disclosed embodiments. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### CITATIONS FOR REFERENCES REFERRED TO IN THE SPECIFICATION

Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res, 25(17), 3389-3402.
Eisen, P. T. Spellman, P. O. Brown, D. Botstein, Cluster analysis and display of genome-wide expression patterns. Proc Natl Acad Sci USA 95, 14863-14868 (1998).
Hornbeck, P. V., Chabra, I., Kornhauser, J. M., Skrzypek, E., and Zhang, B. (2004). PhosphoSite: A bioinformatics resource dedicated to physiological protein phosphorylation. Proteomics, 4(6), 1551-61.
Houseman, B. T. and Mrksich, M. (2002). Towards quantitative assays with peptide chips: a surface engineering approach. Trends Biotechnol, 20(7), 279-81.
Houseman, B. T., Huh, J. H., Kron, S. J., and Mrksich, M. (2002). Peptide chips for the quantitative evaluation of protein kinase activity. Nat Biotechnol, 20(3), 270-4.
Jalal, S., Arsenault, R., Potter, A. A., Babiuk, L. A., Griebel, P. J., and Napper, S. (2009). Genome to kinome: species-specific peptide arrays for kinome analysis. Sci Signal, 2(54), pl1.
Johnson, S. A. and Hunter, T. (2005). Kinomics: methods for deciphering the kinome. Nat Methods, 2(1), 17-25.
Kemp, B. E., Graves, D. J., Benjamini, E., and Krebs, E. G. (1977). Role of multiple basic residues in determining the substrate specificity of cyclic AMP-dependent protein kinase. J Biol Chem, 252(14), 4888-94.
Kersey, P. J., Duarte, J., Williams, A., Karavidopoulou, Y., Birney, E., and Apweiler, R. (2004). The International Protein Index: an integrated database for proteomics experiments. Proteomics, 4(7), 1985-8.
Li Y, Arsenault R J, Trost B, Slind J, Griebel P J, Napper S, Kusalik A. Sci Signal. 2012 Apr. 17; 5(220):p 12
Lynn, D. J., G. L. Winsor, C. Chan, N. Richard, M. R. Laird, A. Barsky, J. L. Gardy, F. M. Roche, T. H. Chan, N. Shah, R. Lo, M. Naseer, J. Que, M. Yau, M. Acab, D. Tulpan, M. D. Whiteside, A. Chikatamarla, B. Mah, T. Munzner, K. Hokamp, R. E. Hancock, F. S. Brinkman, InnateDB: facilitating systems-level analyses of the mammalian innate immune response. Mol Syst Biol 4, 218 (2008).
L″owenberg, M., Tuynman, J., Bilderbeek, J., Gaber, T., Buttgereit, F., van Deventer, S., Peppelenbosch, M., and Hommes, D. (2005). Rapid immunosuppressive effects of glucocorticoids mediated through Lck and Fyn. Blood, 106(5), 1703-10.
Pearson, K Mathematical contributions to the theory of evolution. III. Regression, heredity and panmixia. Philos Trans Royal Soc London Ser A 187, 253-318 (1996).
Schrage, Y. M., Briaire-de Bruijn, I. H., de Miranda, N. F. C. C., van Oosterwijk, J., Taminiau, A. H. M., van Wezel, T., Hogendoorn, P. C. W., and Bov′ee, J. V. M. G. (2009). Kinome profiling of chondrosarcoma reveals SRC-pathway activity and dasatinib as option for treatment. Cancer Res, 69(15), 6216-22.
Sikkema, A. H., Diks, S. H., den Dunnen, W. F. A., ter Elst, A., Scherpen, F. J. G., Hoving, E. W., Ruijtenbeek, R., Boender, P. J., de Wijn, R., Kamps, W. A., Peppelenbosch, M. P., and de Bont, E. S. J. M. (2009). Kinome profiling in pediatric brain tumors as a new approach for target discovery. Cancer Res, 69(14), 5987-95.
Stajich, J. E., Block, D., Boulez, K., Brenner, S. E., Chervitz, S. A., Dagdigian, C., Fuellen, G., Gilbert, J. G. R., Korf, I., Lapp, H., Lehv\ddot{}aslaiho, H., Matsalla, C., Mungall, C. J., Osborne, B. I., Pocock, M. R., Schattner, P., Senger, M., Stein, L. D., Stupka, E., Wilkinson, M. D., and Birney, E. (2002). The Bioperl toolkit: Perl modules for the life sciences. Genome Res, 12(10), 1611-8.
Zetterqvist, O., Ragnarsson, U., Humble, E., Berglund, L., and Engstr\ddot{}om, L. (1976). The minimum substrate of cyclic AMP-stimulated protein kinase, as studied by synthetic peptides representing the phosphorylatable site of pyruvate kinase (type L) of rat liver. Biochem Biophys Res Commun, 70(3), 696-703.

1. A method of preparing one or more species-specific phosphorylation site database entries for a target organism comprising:
a) selecting a first known non-target organism (NTO) phosphorylation site sequence of a first non-target organism, the first known NTO phosphorylation site sequence comprising at least 5 residues and less than 30 residues;
b) obtaining for the first known NTO phosphorylation site sequence a first cognate known NTO phosphorylation polypeptide sequence corresponding to the first known NTO phosphorylation site sequence, the cognate known NTO phosphorylation polypeptide sequence comprising the first known NTO phosphorylation site sequence;
c) identifying a matching target organism (TO) phosphorylation site sequence for the first known NTO phosphorylation site sequence;
d) obtaining for the matching TO phosphorylation site sequence a cognate TO phosphorylation polypeptide sequence corresponding to the matching TO phosphorylation site sequence, the cognate TO phosphorylation polypeptide sequence comprising the matching TO phosphorylation site sequence;
e) determining a plurality of output values, each output value indicative of a degree of matching between the TO phosphorylation site sequence and the NTO phosphorylation site sequence; and
f) determining a similarity value between the first known NTO phosphorylation polypeptide sequence and the cognate TO phosphorylation polypeptide sequence, wherein the similarity value provides an indication of whether the first known NTO phosphorylation polypeptide sequence and the cognate TO phosphorylation polypeptide sequence are orthologues of each other.

2. The method of claim 1, wherein identifying a matching TO phosphorylation site sequence comprises:
a) retrieving a proteome of the target organism;
b) creating a dataset of target organism polypeptide sequences using the retrieved proteome of the target organism; and
c) querying the dataset of target organism polypeptide sequences;
optionally wherein a processor executes a software program to retrieve the proteome of the target organism from an electronic database of protein sequence data and wherein the dataset of proteins of the target organism is a BLAST database created using the makeblastdb program.

3. (canceled)

4. The method of claim 1, wherein the identifying of the matching TO phosphorylation site sequence comprises:
a) i) comparing the first known NTO phosphorylation site sequence against a plurality of sequences of residues of the dataset of target organism polypeptide sequences; and
ii) determining the sequence of the plurality of sequences of residues of the dataset of target organism proteins having the most number of identical residues as the NTO phosphorylation site sequences as the matching TO phosphorylation site sequence; and/or
b) running a blastp search using the first known NTO phosphorylation site sequence as the query and the dataset of target organism proteins as the queried database.

5. (canceled)

6. The method of claim 4, wherein the plurality of output values comprises one or more of: a sequence difference, a non-conservative sequence difference, a matching TO phosphorylation site, a 9-mer sequence difference, and a 9-mer non-conservative sequence difference; optionally
wherein the sequence difference is equal to the difference between the number of residues in the first known NTO phosphorylation site sequence and the number of identical residues between the first known NTO phosphorylation site sequence and the matching TO phosphorylation site sequence;
wherein the non-conservative sequence difference is equal to the difference between the number of residues in the first known NTO phosphorylation site sequence and the sum of the number of identical residues between the first known NTO phosphorylation site sequence and the hit sequence and the number of residues of the hit sequence that are conservative substitutions of the corresponding residue of the first known NTO phosphorylation site sequence;
wherein the matching TO phosphorylation site corresponds to a start position of the TO phosphorylation site sequence in the cognate TO phosphorylation polypeptide sequence;
wherein the 9-mer sequence difference is equal to the number of sequence differences in the count of positions where the two residues are different in a gapless alignment between a 9-amino-acid long peptide corresponding to the first known NTO phosphorylation site sequence and a 9-amino-acid long peptide corresponding to the matching TO phosphorylation site sequence;
and wherein the 9-mer non-conservative sequence difference is equal to the number of non-conservative sequence differences in the count of positions where the two residues have a non-positive score in a gapless alignment between the 9-amino-acid long peptide corresponding to the first known NTO phosphorylation site sequence and the 9-amino-acid long peptide corresponding to the matching TO phosphorylation site sequence.

7. (canceled)

8. The method of claim 6, wherein the plurality of output values further comprises one or more of: a first known NTO phosphorylation site sequence accession number, a first known NTO phosphorylation site sequence description, an indication of whether the first known NTO phosphorylation polypeptide sequence and the cognate TO phosphorylation polypeptide sequence are orthologues of each other, the residues of the first known NTO phosphorylation site sequence, a first known NTO phosphorylation site, a matching TO phosphorylation site sequence accession number, matching TO phosphorylation site sequence description, the residues of the matching TO phosphorylation site sequence, a cognate TO phosphorylation polypeptide sequence rank, matching TO phosphorylation site sequence similarity value, one or more first known NTO phosphorylation site sequence low-throughput references, and one or more first known NTO phosphorylation site sequence high-throughput references.

9. The method of claim 2, wherein the determining of the similarity value comprises:
a) retrieving a proteome of the first known non-target organism;
b) creating a dataset of first known NTO phosphorylation polypeptide sequences using the retrieved non-target organism proteome;
c) comparing the first known NTO phosphorylation polypeptide sequence to each of the TO phosphorylation polypeptide sequences of the dataset of TO phosphorylation polypeptide sequences to generate a plurality of TO dataset similarity values;
d) identifying a best TO dataset similarity value (E¹_{B}) from the plurality of TO dataset similarity values and identifying a first TO dataset similarity value (E¹_{F}) of the match between the first known NTO phosphorylation polypeptide sequence (Q_{F}) and the cognate TO phosphorylation polypeptide sequence (H_{F}) from the plurality of TO dataset similarity values;
e) comparing the TO phosphorylation polypeptide sequence to each of the first known NTO phosphorylation polypeptide sequences in the dataset of first known NTO phosphorylation polypeptide sequences to generate a plurality of NTO dataset similarity values;
f) identifying a best NTO dataset similarity value (E²_{B}) from the plurality of NTO dataset similarity values and identifying a first NTO dataset similarity value (E²_{F}) of the match between the first known NTO phosphorylation polypeptide sequence (Q_{F}) and the cognate TO phosphorylation polypeptide sequence (H_{F}) from the plurality of NTO dataset similarity values; and
g) if the first TO dataset similarity value equals the best TO dataset similarity value and if the first NTO dataset similarity value equals the best NTO dataset similarity value, determining the first known NTO phosphorylation polypeptide sequence and the cognate TO phosphorylation polypeptide sequence are orthologues of each other.

10. The method of claim 1, wherein one or more of the similarity values comprise an E-value; optionally wherein the E-value is selected at less than 10⁻³

11. (canceled)

12. The method of claim 9, wherein:
identifying the best TO similarity value comprises running a blastp search using the first cognate known NTO phosphorylation polypeptide sequence as the query and the dataset of TO proteins as the queried database to generate a plurality of TO dataset E-values, wherein the smallest E-value of the plurality of TO dataset E-values is identified as a best TO dataset E-value;
identifying the best NTO dataset similarity value comprises running a blastp search using the cognate TO phosphorylation polypeptide sequence as the query and the dataset of NTO proteins as the queried database to generate a plurality of NTO dataset E-values, wherein the smallest E-value of the plurality of NTO dataset E-values is identified as a best NTO dataset E-value; and
if the first TO dataset E-value equals the best TO dataset E-value and if the first NTO dataset E-value equals the best NTO dataset E-value, determining the first known NTO phosphorylation polypeptide sequence and the cognate TO phosphorylation polypeptide sequence are orthologues of each other.

13. The method of claim 1, wherein the sequences are in FASTA format, the NTO phosphorylation site sequences are obtained from PhosphoSitePlus data files, the non-target organism polypeptide sequence is a full length protein; and/or each of the first known NTO phosphorylation site sequence and the matching TO phosphorylation site sequence is at least 8 residues and less than or equal to 15 residues in length.

14.-16. (canceled)

17. The method of claim 1, wherein the similarity value is also outputted; and/or the plurality of output values is displayed, optionally wherein the plurality of output values is outputted electronically in a delimited plain text format.

18. (canceled)

19. (canceled)

20. A method of making a species-specific array comprising selecting a plurality of matching target organism phosphorylation site sequences according to the method of claim 4, synthesizing a plurality of peptides each peptide comprising a sequence of one of the matching target organism phosphorylation site sequences and attaching the plurality of peptides to a substrate surface.

21. (canceled)

22. (canceled)

23. A species-specific array comprising a support and a plurality of peptides attached to the support surface, each peptide comprising a sequence of about 5 to about 100 amino acids, for example about 5 to about 50 amino acids or about 5 to about 30 amino acids or about 8 to about 15 amino acids, wherein the sequence is a matching target organism phosphorylation site sequence selected according to claim 1, wherein the similarity is below a preselected threshold; optionally wherein the plurality of peptides comprises at least 100, 200, or 292 matching target organism phosphorylation site sequences; and/or further comprising one or more negative control peptides and/or one or more positive control peptide.

24. (canceled)

25. (canceled)

26. The array of claim 23, wherein the array is a chicken species array and the plurality of peptides are chicken peptides; optionally wherein the plurality of peptides comprises about 5, 10, 15, 20, 25, 50, 75, 100, 125, 150, 175, 200, 225, 250, 275 or 292 peptides each comprising all or part of a sequence selected from SEQ ID NO: 1-292; optionally wherein each peptide is 8-15 contiguous amino acids of a sequence selected from SEQ ID NO: 1-292; and/or wherein each peptide of the plurality of peptides is spotted in replicates of 2, 3, 4, 5, 6, 7, 8, or 9 or more.

27.-29. (canceled)

30. A method of determining kinome activity of a test sample comprising:
a) incubating a species-specific array of claim 23 with the test sample to provide a test array and optionally incubating a second array of claim 23 with a comparator sample to provide a comparator array; and
b) measuring a phosphorylation level signal intensity for each of the plurality of peptides for the test array and optionally the comparator array wherein the phosphorylation level signal intensity results from the interaction of the sample with each of the plurality of peptides;
wherein the kinome activity is determined by identifying an increased or decreased phosphorylation level of one or more of the plurality of peptides on the test array compared to the comparator or an internal control.

31. A method of determining a phosphorylation profile of a test sample comprising:
a) incubating a species-specific array claim 23 with the test sample to provide a test array; and
b) measuring a phosphorylation level signal intensity for each of the plurality of peptides for the test array to provide a test array phosphorylation profile, wherein the phosphorylation level signal intensity results from the interaction of the sample with each of the plurality of peptides.

32. The method of claim 31 further comprising incubating a species-specific array with a comparator sample to provide a comparator array; measuring a phosphorylation level signal intensity for each of the plurality of peptides for the comparator array to provide a comparator phosphorylation profile, wherein the phosphorylation level signal intensity results from the interaction of the sample with each of the plurality of peptides; and comparing the test array phosphorylation profile to the comparator phosphorylation profile to detect one or more differentially phosphorylated peptides.

33. A non-transitory computer-readable storage medium upon which a plurality of instructions are stored, the instructions for performing the steps of:
a) querying a dataset comprising a plurality of target organism (TO) polypeptide sequences with a selected plurality of known NTO phosphorylation site sequences (query phosphorylation site sequences) to identify for each of the plurality a matching TO phosphorylation site sequence;
b) obtaining for each of the matching TO phosphorylation site sequences a cognate TO phosphorylation polypeptide sequence corresponding to the matching TO phosphorylation site sequence, the cognate TO phosphorylation polypeptide sequence comprising the matching TO phosphorylation site sequence;
c) determining a plurality of output values, one or more of the output values being indicative of a degree of matching between the TO phosphorylation site sequence and the NTO phosphorylation site sequence; and
d) determining a similarity value between the first known NTO phosphorylation polypeptide sequence and the cognate TO phosphorylation polypeptide sequence, wherein the similarity value provides an indication of whether the first known NTO phosphorylation polypeptide sequence and the cognate TO phosphorylation polypeptide sequence are orthologues of each other.

34. The non-transitory computer-readable storage medium of claim 33 wherein the instructions are further for performing the step of displaying the matching TO phosphorylation site sequences and/or cognate TO sequence accession numbers when the similarity value is below a preselected threshold; optionally wherein the similarity value is an E-value and the preselected threshold is 10⁻³.

35. (canceled)

36. A non-transitory computer-readable storage medium upon which a plurality of instructions are stored, wherein the instructions are for performing the steps of the method as claimed in claim 1.

37. A system for preparing one or more species-specific phosphorylation site database entries for a target organism, the system comprising:
a) a memory for storing a plurality of instructions; and
b) a processor coupled to the memory for:
i) obtaining for a first known non-target organism (NTO) phosphorylation site sequence of a first non-target organism, the first known NTO phosphorylation site sequence comprising at least 5 residues and less than 30 residues, a first cognate known NTO phosphorylation polypeptide sequence corresponding to the first known NTO phosphorylation site sequence, the cognate known NTO phosphorylation polypeptide sequence comprising the first known NTO phosphorylation site sequence;
ii) identifying a matching target organism (TO) phosphorylation site sequence for the first known NTO phosphorylation site sequence;
iii) obtaining for the matching TO phosphorylation site sequence a cognate TO phosphorylation polypeptide sequence corresponding to the matching TO phosphorylation site sequence, the cognate TO phosphorylation polypeptide sequence comprising the matching TO phosphorylation site sequence;
iv) determining a plurality of output values, one or more of the output values being indicative of a degree of matching between the TO phosphorylation site sequence and the NTO phosphorylation site sequence; and
v) determining a similarity value between the first known NTO phosphorylation polypeptide sequence and the cognate TO phosphorylation polypeptide sequence, wherein the similarity value provides an indication of whether the first known NTO phosphorylation polypeptide sequence and the cognate TO phosphorylation polypeptide sequence are orthologues of each other.

38. A kit comprising,
i) a plurality of peptides of claim 21,
ii) an array comprising said plurality of peptides;
iii) optionally i) or ii) in combination with a kit control; and
iv) a package housing the peptides, array and/or kit control.


SEQUENCE LISTING (292 sequences)

SEQ ID NO: 1 - protein, 15 aa, Gallus gallus
AADESVGTMGNRLQR

SEQ ID NO: 2 - protein, 15 aa, Gallus gallus
ADTLKERYQKIGDTK

SEQ ID NO: 3 - protein, 15 aa, Gallus gallus
AEIGEGAYGKVFKAR

SEQ ID NO: 4 - protein, 15 aa, Gallus gallus
AEKGVPLYRHIADLA

SEQ ID NO: 5 - protein, 15 aa, Gallus gallus
AEPGSNVYLRRELIC

SEQ ID NO: 6 - protein, 15 aa, Gallus gallus
AGKASFAYAWVLDET

SEQ ID NO: 7 - protein, 15 aa, Gallus gallus
AGVMITASHNRKEDN

SEQ ID NO: 8 - protein, 15 aa, Gallus gallus
AKEIDVSFVKIEEVI

SEQ ID NO: 9 - protein, 15 aa, Gallus gallus
AKNAVEEYVYDFRDK

SEQ ID NO: 10 - protein, 15 aa, Gallus gallus
ALRNRSNTPILVDGK

SEQ ID NO: 11 - protein, 15 aa, Gallus gallus
ALSDHHVYLEGTLLK

SEQ ID NO: 12 - protein, 15 aa, Gallus gallus
APQIQDLYGKVDFTE

SEQ ID NO: 13 - protein, 15 aa, Gallus gallus
AQNKLSLTQDPVVKV

SEQ ID NO: 14 - protein, 15 aa, Gallus gallus
AQQCNGIYIWKIENF

SEQ ID NO: 15 - protein, 15 aa, Gallus gallus
ARQSRRSTQGVTLTD

SEQ ID NO: 16 - protein, 14 aa, Gallus gallus
ATKIALYETPTGWK

SEQ ID NO: 17 - protein, 15 aa, Gallus gallus
ATPQRSGSVSNYRSC

SEQ ID NO: 18 - protein, 15 aa, Gallus gallus
ATYIAGLSGSTWYMS

SEQ ID NO: 19 - protein, 15 aa, Gallus gallus
AVKLRGRSFQNNWNV

SEQ ID NO: 20 - protein, 15 aa, Gallus gallus
CADVPLLTPSSKEMM

SEQ ID NO: 21 - protein, 15 aa, Gallus gallus
CASDGKSYDNACQIK

SEQ ID NO: 22 - protein, 15 aa, Gallus gallus
CNENFKKTFKKILHI

SEQ ID NO: 23 - protein, 15 aa, Gallus gallus
CTMSVDRYVAVCHPV

SEQ ID NO: 24 - protein, 15 aa, Gallus gallus
DDTSDPTYTSSLGGK

SEQ ID NO: 25 - protein, 14 aa, Gallus gallus
DGATMKTFCGTPEY

SEQ ID NO: 26 - protein, 15 aa, Gallus gallus
DGSFIGQYSGKKEKE

SEQ ID NO: 27 - protein, 15 aa, Gallus gallus
DGVVGKSSDGEDEQQ

SEQ ID NO: 28 - protein, 15 aa, Gallus gallus
DKYFDEQYEYRHVML

SEQ ID NO: 29 - protein, 15 aa, Gallus gallus
DMSELSSSPPGPYHQ

SEQ ID NO: 30 - protein, 15 aa, Gallus gallus
DPFIDLNYMVYMFKY

SEQ ID NO: 31 - protein, 15 aa, Gallus gallus
DRASHASSSDWTPRP

SEQ ID NO: 32 - protein, 15 aa, Gallus gallus
DRGYISPYFINTAKG

SEQ ID NO: 33 - protein, 15 aa, Gallus gallus
DSAKGFDYKTCNVLV

SEQ ID NO: 34 - protein, 15 aa, Gallus gallus
DSLPCSPSSATPHSQ

SEQ ID NO: 35 - protein, 16 aa, Gallus gallus
DSREDEISPPPPMNPV

SEQ ID NO: 36 - protein, 15 aa, Gallus gallus
DSVFCPHYEKVSGDY

SEQ ID NO: 37 - protein, 15 aa, Gallus gallus
DYNDGRRTFPRIRRH

SEQ ID NO: 38 - protein, 15 aa, Gallus gallus
EADDWLRYGNPWEKA

SEQ ID NO: 39 - protein, 15 aa, Gallus gallus
EDDEKFVSVYGTEEY

SEQ ID NO: 40 - protein, 15 aa, Gallus gallus
EGVRNIKSMWEKGNV

SEQ ID NO: 41 - protein, 15 aa, Gallus gallus
EKIGEGTYGVVYKAR

SEQ ID NO: 42 - protein, 15 aa, Gallus gallus
EKMISGMYMGELVRL

SEQ ID NO: 43 - protein, 15 aa, Gallus gallus
ELDELMASLSDFKFM

SEQ ID NO: 44 - protein, 15 aa, Gallus gallus
ELVVRDPYALKPIRK

SEQ ID NO: 45 - protein, 15 aa, Gallus gallus
EPDHYRYSDTTDSDP

SEQ ID NO: 46 - protein, 15 aa, Gallus gallus
EPKSPGEYVNIEFGS

SEQ ID NO: 47 - protein, 15 aa, Gallus gallus
EPRSRHLSVSSQNTG

SEQ ID NO: 48 - protein, 15 aa, Gallus gallus
ERTLYRQSLPPLAKL

SEQ ID NO: 49 - protein, 15 aa, Gallus gallus
ESTESSNTTIEDEDV

SEQ ID NO: 50 - protein, 15 aa, Gallus gallus
EVLGRGVSSVVRRCI

SEQ ID NO: 51 - protein, 15 aa, Gallus gallus
EWSCTRCTFLNPVGQ

SEQ ID NO: 52 - protein, 15 aa, Gallus gallus
FCDSPPQSPTFPEAG

SEQ ID NO: 53 - protein, 15 aa, Gallus gallus
FDKDGNGYISAAELR

SEQ ID NO: 54 - protein, 15 aa, Gallus gallus
FERADSEYTDKLQHY

SEQ ID NO: 55 - protein, 15 aa, Gallus gallus
FGLARAFSLAKNSQP

SEQ ID NO: 56 - protein, 15 aa, Gallus gallus
FIRFDKRSEAEEAIT

SEQ ID NO: 57 - protein, 15 aa, Gallus gallus
FTSIGEDYDERVLPS

SEQ ID NO: 58 - protein, 15 aa, Gallus gallus
GDLVIVLTGWRPGSG

SEQ ID NO: 59 - protein, 15 aa, Gallus gallus
GEENAVLYQNYKEKA

SEQ ID NO: 60 - protein, 15 aa, Gallus gallus
GETAKGDYPLEAVRM

SEQ ID NO: 61 - protein, 15 aa, Gallus gallus
GEVQRRLSPPECLNA

SEQ ID NO: 62 - protein, 15 aa, Gallus gallus
GGPEPGPYAQPSINT

SEQ ID NO: 63 - protein, 15 aa, Gallus gallus
GINPCTETFTGTLQY

SEQ ID NO: 64 - protein, 15 aa, Gallus gallus
GIPVRCYSAEVVTLW

SEQ ID NO: 65 - protein, 15 aa, Gallus gallus
GIPVRVYTHEVVTLW

SEQ ID NO: 66 - protein, 15 aa, Gallus gallus
GKGTPLGTPATSPPP

SEQ ID NO: 67 - protein, 15 aa, Gallus gallus
GLKVGVSSRINEWLT

SEQ ID NO: 68 - protein, 15 aa, Gallus gallus
GLSSAMCYSALVTKT

SEQ ID NO: 69 - protein, 15 aa, Gallus gallus
GLSSSPSTPTQVTKQ

SEQ ID NO: 70 - protein, 15 aa, Gallus gallus
GNRTTPSYVAFTDTE

SEQ ID NO: 71 - protein, 15 aa, Gallus gallus
GNWRRGATAGGCRNY

SEQ ID NO: 72 - protein, 15 aa, Gallus gallus
GPEKDHVYLQLHHLP

SEQ ID NO: 73 - protein, 15 aa, Gallus gallus
GPRVWFVSNIDGTHI

SEQ ID NO: 74 - protein, 15 aa, Gallus gallus
GRKRKMRSKKEDSSD

SEQ ID NO: 75 - protein, 15 aa, Gallus gallus
GSPGMKIYIDPFTYE

SEQ ID NO: 76 - protein, 15 aa, Gallus gallus
GSPNRVYTHQVVTRW

SEQ ID NO: 77 - protein, 15 aa, Gallus gallus
GTEPKIKYYSELCAP

SEQ ID NO: 78 - protein, 15 aa, Gallus gallus
GVPVRTYTHEVVTLW

SEQ ID NO: 79 - protein, 15 aa, Gallus gallus
GYNAREYYDRIPELR

SEQ ID NO: 80 - protein, 15 aa, Gallus gallus
HDLKRCQYVTEKVLA

SEQ ID NO: 81 - protein, 15 aa, Gallus gallus
HFDPVTRSPLTQDQL

SEQ ID NO: 82 - protein, 15 aa, Gallus gallus
HKDKFLQTFCGSPLY

SEQ ID NO: 83 - protein, 15 aa, Gallus gallus
HQLFRGFSFVATGLM

SEQ ID NO: 84 - protein, 15 aa, Gallus gallus
HRLRRRGSTVPQFTN

SEQ ID NO: 85 - protein, 15 aa, Gallus gallus
IDNIFRFTQAGSEVS

SEQ ID NO: 86 - protein, 15 aa, Gallus gallus
IEDDIIYTQDFTVPG

SEQ ID NO: 87 - protein, 15 aa, Gallus gallus
IEHIGLLYQEYRDKS

SEQ ID NO: 88 - protein, 15 aa, Gallus gallus
IEKIGEGTYGVVYKG

SEQ ID NO: 89 - protein, 15 aa, Gallus gallus
IEKRYRSSINDKIIE

SEQ ID NO: 90 - protein, 15 aa, Gallus gallus
IERLRTHSIESSGKL

SEQ ID NO: 91 - protein, 15 aa, Gallus gallus
IGVMVTASHNPEEDN

SEQ ID NO: 92 - protein, 15 aa, Gallus gallus
IKLECVKTKHPQLHI

SEQ ID NO: 93 - protein, 15 aa, Gallus gallus
ILHRYYRSPLVQIYE

SEQ ID NO: 94 - protein, 15 aa, Gallus gallus
IPEPAHAYAQPQTTS

SEQ ID NO: 95 - protein, 15 aa, Gallus gallus
IQRIMHNYEKLKSRI

SEQ ID NO: 96 - protein, 15 aa, Gallus gallus
IREAMTAYNSHEEGR

SEQ ID NO: 97 - protein, 15 aa, Gallus gallus
ISEDIKSYYTVRQLE

SEQ ID NO: 98 - protein, 15 aa, Gallus gallus
ISGYLVDSVAKTMDA

SEQ ID NO: 99 - protein, 15 aa, Gallus gallus
ISIRGTLSPKDALTD

SEQ ID NO: 100 - protein, 15 aa, Gallus gallus
KEFGVERSLRPMDSS

SEQ ID NO: 101 - protein, 15 aa, Gallus gallus
KEPTRRFSTIVVEEG

SEQ ID NO: 102 - protein, 15 aa, Gallus gallus
KEREKEISDDEAEEE

SEQ ID NO: 103 - protein, 15 aa, Gallus gallus
KESQKSIYYITGESK

SEQ ID NO: 104 - protein, 14 aa, Gallus gallus
KILEEVRYIANRFR

SEQ ID NO: 105 - protein, 15 aa, Gallus gallus
KISEKKMSTPVEVLC

SEQ ID NO: 106 - protein, 15 aa, Gallus gallus
KKTVMIKTIETRDGE

SEQ ID NO: 107 - protein, 15 aa, Gallus gallus
KLKKEDIYAVEIVGG

SEQ ID NO: 108 - protein, 15 aa, Gallus gallus
KLSLNPIYRQVPRLV

SEQ ID NO: 109 - protein, 15 aa, Gallus gallus
KPGNLLLTTNGTLKI

SEQ ID NO: 110 - protein, 15 aa, Gallus gallus
KPIWQRPSKEVEEDE

SEQ ID NO: 111 - protein, 15 aa, Gallus gallus
KQRRSIISPNFSFMG

SEQ ID NO: 112 - protein, 15 aa, Gallus gallus
KQVVESAYEVIRLKG

SEQ ID NO: 113 - protein, 15 aa, Gallus gallus
KRFSFKKSFKLSGFS

SEQ ID NO: 114 - protein, 15 aa, Gallus gallus
KSDISSSSQGVIEKE

SEQ ID NO: 115 - protein, 15 aa, Gallus gallus
KSFLDSGYRILGAVA

SEQ ID NO: 116 - protein, 15 aa, Gallus gallus
KSIQATLTPSAMKSS

SEQ ID NO: 117 - protein, 15 aa, Gallus gallus
KTLGRRDSSDDWEIP

SEQ ID NO: 118 - protein, 15 aa, Gallus gallus
KVPQRTTSISPALAR

SEQ ID NO: 119 - protein, 15 aa, Gallus gallus
LAREWHKTTKMSAAG

SEQ ID NO: 120 - protein, 15 aa, Gallus gallus
LDAPRLETKSLSSSV

SEQ ID NO: 121 - protein, 15 aa, Gallus gallus
LDGVTTRTFCGTPDY

SEQ ID NO: 122 - protein, 15 aa, Gallus gallus
LERKRPVSMAVMEGD

SEQ ID NO: 123 - protein, 15 aa, Gallus gallus
LFRLEQGFELQFRLG

SEQ ID NO: 124 - protein, 15 aa, Gallus gallus
LGGLRISSDSSSDIE

SEQ ID NO: 125 - protein, 15 aa, Gallus gallus
LKKQAAEYREIDKRM

SEQ ID NO: 126 - protein, 15 aa, Gallus gallus
LMKKELDYFAKALES

SEQ ID NO: 127 - protein, 15 aa, Gallus gallus
LMKTLCGTPTYLAPE

SEQ ID NO: 128 - protein, 15 aa, Gallus gallus
LMTKLRASTTSETIQ

SEQ ID NO: 129 - protein, 15 aa, Gallus gallus
LPLLVQRTIARTIVL

SEQ ID NO: 130 - protein, 15 aa, Gallus gallus
LRRIGRFSEPHARFY

SEQ ID NO: 131 - protein, 15 aa, Gallus gallus
LSDSYSNTLPVRKNV

SEQ ID NO: 132 - protein, 15 aa, Gallus gallus
LVDSIAKTRDAGCRP

SEQ ID NO: 133 - protein, 15 aa, Gallus gallus
LVTSEASYCKSLNLL

SEQ ID NO: 134 - protein, 15 aa, Gallus gallus
MAHKQIYYSDKYDDE

SEQ ID NO: 135 - protein, 15 aa, Gallus gallus
MAMKTKTYQVAQMKS

SEQ ID NO: 136 - protein, 15 aa, Gallus gallus
MKPGEYSYFSPRTLS

SEQ ID NO: 137 - protein, 15 aa, Gallus gallus
MLRTDLSYLCSRWRM

SEQ ID NO: 138 - protein, 15 aa, Gallus gallus
MPPLIADSPKARCPL

SEQ ID NO: 139 - protein, 11 aa, Gallus gallus
MPPSPLDDRVV

SEQ ID NO: 140 - protein, 15 aa, Gallus gallus
MRIGAEVYHNLKNVI

SEQ ID NO: 141 - protein, 15 aa, Gallus gallus
MSGTGIRSVTGTPYW

SEQ ID NO: 142 - protein, 15 aa, Gallus gallus
MTPGMKIYIDPFTYE

SEQ ID NO: 143 - protein, 13 aa, Gallus gallus
MVKETTYYDVLGV

SEQ ID NO: 144 - protein, 15 aa, Gallus gallus
MVQEAEKYKAEDEKQ

SEQ ID NO: 145 - protein, 15 aa, Gallus gallus
NDTGSKYYKEIPLSE

SEQ ID NO: 146 - protein, 15 aa, Gallus gallus
NEYLRSISLPVPVLV

SEQ ID NO: 147 - protein, 15 aa, Gallus gallus
NKPEDCPYLWAHMKK

SEQ ID NO: 148 - protein, 15 aa, Gallus gallus
NLQNGPFYARVIQKR

SEQ ID NO: 149 - protein, 15 aa, Gallus gallus
NPLMRRNSVTPLASP

SEQ ID NO: 150 - protein, 15 aa, Gallus gallus
NQKKRSESFRFQQEN

SEQ ID NO: 151 - protein, 15 aa, Gallus gallus
NQVFLGFTYVAPSVL

SEQ ID NO: 152 - protein, 15 aa, Gallus gallus
NRFTRRASVCAEAYN

SEQ ID NO: 153 - protein, 15 aa, Gallus gallus
NSEESRPYTNKVITL

SEQ ID NO: 154 - protein, 15 aa, Gallus gallus
NSQPNRYTNRVVTLW

SEQ ID NO: 155 - protein, 15 aa, Gallus gallus
PASSAKTSPAKQQAP

SEQ ID NO: 156 - protein, 15 aa, Gallus gallus
PEFRIEDSEPHIPLI

SEQ ID NO: 157 - protein, 15 aa, Gallus gallus
PEGEKLHSDSGISVD

SEQ ID NO: 158 - protein, 15 aa, Gallus gallus
PEPESEESDLEIDNE

SEQ ID NO: 159 - protein, 15 aa, Gallus gallus
PEQSKRSTMVGTPYW

SEQ ID NO: 160 - protein, 15 aa, Gallus gallus
PETEENIYQVPTSQK

SEQ ID NO: 161 - protein, 15 aa, Gallus gallus
PGEDFPASPQRRNTS

SEQ ID NO: 162 - protein, 15 aa, Gallus gallus
PGRMRRSSLTPLAST

SEQ ID NO: 163 - protein, 15 aa, Gallus gallus
PIEQLLDYNRIRSGM

SEQ ID NO: 164 - protein, 15 aa, Gallus gallus
PKIHRSASEPSLNRA

SEQ ID NO: 165 - protein, 15 aa, Gallus gallus
PLCMITEYMENGDLN

SEQ ID NO: 166 - protein, 15 aa, Gallus gallus
PNRQRIRSCVSAENF

SEQ ID NO: 167 - protein, 15 aa, Gallus gallus
PPPRSHVSMVDPNES

SEQ ID NO: 168 - protein, 15 aa, Gallus gallus
PPSREAQYNNFAGNS

SEQ ID NO: 169 - protein, 15 aa, Gallus gallus
PREKRRSTGVSFWTQ

SEQ ID NO: 170 - protein, 15 aa, Gallus gallus
PRPEHTKSIYTRSVI

SEQ ID NO: 171 - protein, 15 aa, Gallus gallus
PTVIHKHYQIHRIRQ

SEQ ID NO: 172 - protein, 15 aa, Gallus gallus
PVFDLTATPKGGTPA

SEQ ID NO: 173 - protein, 14 aa, Gallus gallus
QAASNFKSPVKTIR

SEQ ID NO: 174 - protein, 15 aa, Gallus gallus
QAQPRQDYLKGLSII

SEQ ID NO: 175 - protein, 15 aa, Gallus gallus
QAQRFRFSPMGVDHM

SEQ ID NO: 176 - protein, 15 aa, Gallus gallus
QDNDQPDYDSVASDE

SEQ ID NO: 177 - protein, 15 aa, Gallus gallus
QELHDIHSTRSKERL

SEQ ID NO: 178 - protein, 15 aa, Gallus gallus
QGTGTNGSEISDSDY

SEQ ID NO: 179 - protein, 15 aa, Gallus gallus
QLVKMLLYTEVTRYL

SEQ ID NO: 180 - protein, 15 aa, Gallus gallus
QMVNGAHSSSTLDEA

SEQ ID NO: 181 - protein, 15 aa, Gallus gallus
QNTRDHASTANTVDR

SEQ ID NO: 182 - protein, 15 aa, Gallus gallus
QRQRSTSTPNVHMVS

SEQ ID NO: 183 - protein, 15 aa, Gallus gallus
QSDFEGFSYVNPQFV

SEQ ID NO: 184 - protein, 15 aa, Gallus gallus
QVKIWRRSFDIPPPP

SEQ ID NO: 185 - protein, 15 aa, Gallus gallus
RAIGRLSSMAMISGM

SEQ ID NO: 186 - protein, 15 aa, Gallus gallus
RAVRRLRTACERAKR

SEQ ID NO: 187 - protein, 15 aa, Gallus gallus
RDVYDKEYYSVHNKT

SEQ ID NO: 188 - protein, 15 aa, Gallus gallus
RFHGRAFSDPFVQAE

SEQ ID NO: 189 - protein, 15 aa, Gallus gallus
RGAPVNVSSSDLTGR

SEQ ID NO: 190 - protein, 15 aa, Gallus gallus
RGEPNVSYICSRYYR

SEQ ID NO: 191 - protein, 15 aa, Gallus gallus
RKMKDTDSEEEIREA

SEQ ID NO: 192 - protein, 15 aa, Gallus gallus
RLDGENIYIRHSNLM

SEQ ID NO: 193 - protein, 15 aa, Gallus gallus
RLLAGPDTDVLSFVL

SEQ ID NO: 194 - protein, 15 aa, Gallus gallus
RNGRKHASILLRKKD

SEQ ID NO: 195 - protein, 15 aa, Gallus gallus
RPHFPQFSYSASGTA

SEQ ID NO: 196 - protein, 15 aa, Gallus gallus
RPPGRPISGHGMDSR

SEQ ID NO: 197 - protein, 15 aa, Gallus gallus
RPRGQRDSSYYWEIE

SEQ ID NO: 198 - protein, 15 aa, Gallus gallus
RREDKYMYFEFPQPL

SEQ ID NO: 199 - protein, 14 aa, Gallus gallus
RREERSLSAPGNLL

SEQ ID NO: 200 - protein, 15 aa, Gallus gallus
RREERSMSAPGNLLI

SEQ ID NO: 201 - protein, 15 aa, Gallus gallus
RRLLFYKYVYKKYRA

SEQ ID NO: 202 - protein, 15 aa, Gallus gallus
RRSDNEEYVEVGRLG

SEQ ID NO: 203 - protein, 15 aa, Gallus gallus
RSQELRKTFKEIICC

SEQ ID NO: 204 - protein, 15 aa, Gallus gallus
RSRTRTDSYSASQSV

SEQ ID NO: 205 - protein, 15 aa, Gallus gallus
RTHFPQFSYSASIRE

SEQ ID NO: 206 - protein, 15 aa, Gallus gallus
RVEAMKQYQEEIQEL

SEQ ID NO: 207 - protein, 15 aa, Gallus gallus
RVKGRTWTLCGTPEY

SEQ ID NO: 208 - protein, 15 aa, Gallus gallus
RVYAEVNSLRSREYW

SEQ ID NO: 209 - protein, 15 aa, Gallus gallus
RYMEDSTYYKASKGK

SEQ ID NO: 210 - protein, 15 aa, Gallus gallus
RYPGGESYQDLVQRL

SEQ ID NO: 211 - protein, 15 aa, Gallus gallus
SAGDKVYTVEKADNF

SEQ ID NO: 212 - protein, 15 aa, Gallus gallus
SAVNSRETMFHKERF

SEQ ID NO: 213 - protein, 15 aa, Gallus gallus
SCMHRQETVDCLKKF

SEQ ID NO: 214 - protein, 15 aa, Gallus gallus
SDDFDSDYENPDGHS

SEQ ID NO: 215 - protein, 15 aa, Gallus gallus
SDGATMKTFCGTPEY

SEQ ID NO: 216 - protein, 15 aa, Gallus gallus
SDGEFLRTSCGSPNY

SEQ ID NO: 217 - protein, 15 aa, Gallus gallus
SGASTGIYEALELRD

SEQ ID NO: 218 - protein, 15 aa, Gallus gallus
SGISSVPTPSPLGPL

SEQ ID NO: 219 - protein, 15 aa, Gallus gallus
SGRDLSSSPPGPYGQ

SEQ ID NO: 220 - protein, 15 aa, Gallus gallus
SGRKPMLYSFQTSLP

SEQ ID NO: 221 - protein, 15 aa, Gallus gallus
SGRPRTTSFAESCKP

SEQ ID NO: 222 - protein, 15 aa, Gallus gallus
SIWKGVKTSGKVVWV

SEQ ID NO: 223 - protein, 15 aa, Gallus gallus
SKIPLTRSHNNFVAI

SEQ ID NO: 224 - protein, 15 aa, Gallus gallus
SKRHQKFTHFLPRPV

SEQ ID NO: 225 - protein, 15 aa, Gallus gallus
SKVKRQSSTPNASEL

SEQ ID NO: 226 - protein, 15 aa, Gallus gallus
SLPLTPESPNDPKGS

SEQ ID NO: 227 - protein, 15 aa, Gallus gallus
SMMHRQETVECLKKF

SEQ ID NO: 228 - protein, 15 aa, Gallus gallus
SMMHRQETVECLRKF

SEQ ID NO: 229 - protein, 15 aa, Gallus gallus
SPIEKVLSPLRSPPL

SEQ ID NO: 230 - protein, 15 aa, Gallus gallus
SQGGEPTYNVAVGRA

SEQ ID NO: 231 - protein, 15 aa, Gallus gallus
SQITSQVTGQIGWRR

SEQ ID NO: 232 - protein, 15 aa, Gallus gallus
SQKKEGVYDVPKSQP

SEQ ID NO: 233 - protein, 15 aa, Gallus gallus
SQPYSARSRLSAMEI

SEQ ID NO: 234 - protein, 15 aa, Gallus gallus
SQQGMTVYGLPRQVY

SEQ ID NO: 235 - protein, 15 aa, Gallus gallus
SQRQRSTSTPNVHMV

SEQ ID NO: 236 - protein, 15 aa, Gallus gallus
SQSGMTAYGTRRHLY

SEQ ID NO: 237 - protein, 15 aa, Gallus gallus
SREYDRLYEDYTRTS

SEQ ID NO: 238 - protein, 15 aa, Gallus gallus
SRLFMHPYELMAKVC

SEQ ID NO: 239 - protein, 15 aa, Gallus gallus
SRQARANSFVGTAQY

SEQ ID NO: 240 - protein, 15 aa, Gallus gallus
SSGSPANSFHFKEAW

SEQ ID NO: 241 - protein, 13 aa, Gallus gallus
SSKIRKLSTCKQQ

SEQ ID NO: 242 - protein, 15 aa, Gallus gallus
STFDAHIYEGRVIQI

SEQ ID NO: 243 - protein, 15 aa, Gallus gallus
STPRRSDSAISVRSL

SEQ ID NO: 244 - protein, 14 aa, Gallus gallus
SVSDQFSVEFEVES

SEQ ID NO: 245 - protein, 15 aa, Gallus gallus
SVSETDDYAEIIDEE

SEQ ID NO: 246 - protein, 15 aa, Gallus gallus
TAKTPKDSPGIPPSA

SEQ ID NO: 247 - protein, 15 aa, Gallus gallus
TDDEMTGYVATRWYR

SEQ ID NO: 248 - protein, 15 aa, Gallus gallus
TDGKKVYYPADPVPY

SEQ ID NO: 249 - protein, 15 aa, Gallus gallus
TGKENKITITNDKGR

SEQ ID NO: 250 - protein, 15 aa, Gallus gallus
TGMFPRNYVTPVNRN

SEQ ID NO: 251 - protein, 15 aa, Gallus gallus
THLAWINTPRKQGGL

SEQ ID NO: 252 - protein, 15 aa, Gallus gallus
THSRIEQYATRLAQM

SEQ ID NO: 253 - protein, 15 aa, Gallus gallus
TKIPLIKSHNDFVAI

SEQ ID NO: 254 - protein, 15 aa, Gallus gallus
TKSIYTRSVIDPIPA

SEQ ID NO: 255 - protein, 15 aa, Gallus gallus
TPPRRAPSPDGFSPY

SEQ ID NO: 256 - protein, 15 aa, Gallus gallus
TRGQPVLTPPDQLVI

SEQ ID NO: 257 - protein, 15 aa, Gallus gallus
TVGNKLDTFCGSPPY

SEQ ID NO: 258 - protein, 15 aa, Gallus gallus
TVPESIHSFIGDGLV

SEQ ID NO: 259 - protein, 15 aa, Gallus gallus
TVQNALQTPCYTPYY

SEQ ID NO: 260 - protein, 15 aa, Gallus gallus
TYIDPHTYEDPNQAV

SEQ ID NO: 261 - protein, 15 aa, Gallus gallus
VFDLGGGTFDVSLLT

SEQ ID NO: 262 - protein, 15 aa, Gallus gallus
VIGIDLGTTNSCVAV

SEQ ID NO: 263 - protein, 15 aa, Gallus gallus
VIRLKGYTNWAIGLS

SEQ ID NO: 264 - protein, 15 aa, Gallus gallus
VKILTGFYQDFEKIS

SEQ ID NO: 265 - protein, 15 aa, Gallus gallus
VLDIEQFSTVKGVNL

SEQ ID NO: 266 - protein, 15 aa, Gallus gallus
VLGTDELYGYLKKYH

SEQ ID NO: 267 - protein, 15 aa, Gallus gallus
VLNTHRKSLNLVDIP

SEQ ID NO: 268 - protein, 15 aa, Gallus gallus
VLSSRKLSLQERSSG

SEQ ID NO: 269 - protein, 15 aa, Gallus gallus
VLVRHGESAWNLENR

SEQ ID NO: 270 - protein, 15 aa, Gallus gallus
VPVEITISLLKRAMD

SEQ ID NO: 271 - protein, 15 aa, Gallus gallus
VSGQLIDSMANSFVG

SEQ ID NO: 272 - protein, 15 aa, Gallus gallus
VSTQLVNSIAKTYVG

SEQ ID NO: 273 - protein, 15 aa, Gallus gallus
VTVSLSLTAKRMAKK

SEQ ID NO: 274 - protein, 15 aa, Gallus gallus
VWDHIEVSDDEDETH

SEQ ID NO: 275 - protein, 15 aa, Gallus gallus
VYIDPFTYEDPNEAV

SEQ ID NO: 276 - protein, 15 aa, Gallus gallus
WEQGQADYMGVDSFD

SEQ ID NO: 277 - protein, 15 aa, Gallus gallus
WGLNKQGYKCRQCNA

SEQ ID NO: 278 - protein, 15 aa, Gallus gallus
WNLENRFCGWYDADL

SEQ ID NO: 279 - protein, 15 aa, Gallus gallus
WRLNERHYGALTGLN

SEQ ID NO: 280 - protein, 15 aa, Gallus gallus
WSKVVLAYEPVWAIG

SEQ ID NO: 281 - protein, 9 aa, Gallus gallus
WVRKTPWYQ

SEQ ID NO: 282 - protein, 15 aa, Gallus gallus
WYDNEFGYSNRVVDL

SEQ ID NO: 283 - protein, 15 aa, Gallus gallus
YDWMRRVTQRKKISK

SEQ ID NO: 284 - protein, 15 aa, Gallus gallus
YIEDEEYYKASVTRL

SEQ ID NO: 285 - protein, 15 aa, Gallus gallus
YIGNLNESVTPADLE

SEQ ID NO: 286 - protein, 15 aa, Gallus gallus
YIQEVVQYIKRLEDA

SEQ ID NO: 287 - protein, 15 aa, Gallus gallus
YQRSKSLSPSQLGYQ

SEQ ID NO: 288 - protein, 15 aa, Gallus gallus
YSFQMALTSVVVTLW

SEQ ID NO: 289 - protein, 15 aa, Gallus gallus
YSHKGHLSEGLVTKW

SEQ ID NO: 290 - protein, 15 aa, Gallus gallus
YSLQISSIPLYKKKE

SEQ ID NO: 291 - protein, 15 aa, Gallus gallus
YSSSQRVSSYRRTFG

SEQ ID NO: 292 - protein, 15 aa, Gallus gallus
YVHVNATYVNVKCVA